(19) United States
(12) Patent Application Publication
(10) Pub. No.: US 2019/0016746 A1
(43) Pub. Date: Jan. 17, 2019

(54) AMINOGLYCOSIDE DERIVATIVES AND USES THEREOF IN TREATING GENETIC DISORDERS

(71) Applicant: Eloxx Pharmaceuticals Ltd., Rehovot (IL)

(72) Inventors: Timor BAASOV, Haifa (IL); Shmuel TUVIA, Natania (IL)

(73) Assignee: Eloxx Pharmaceuticals Ltd., Rehovot (IL)

(21) Appl. No.: 16/068,165

(22) PCT Filed: Sep. 2, 2016
(86) PCT No.: PCT/IL2016/050969

§ 371 Date: Jul. 5, 2018

Related U.S. Application Data
(60) Provisional application No. 62/274,915, filed on Jan. 5, 2016.

(51) Int. Cl. C07H 15/23; C07H 15/224

(52) U.S. Cl. CPC C07H 15/23; C07H 15/224

(57) ABSTRACT

Novel aminoglycosides, represented by Formulae I, Ia, III and IIIa, as defined in the instant specification, designed to exhibit stop codon mutation readthrough activity, are provided. Also provided are pharmaceutical compositions containing the same, and uses thereof in the treatment of genetic diseases and disorders, such as diseases and disorders associated with stop codon mutations.

### FIELD AND BACKGROUND OF THE INVENTION

The present invention, in some embodiments thereof, relates to aminoglycosides and more particularly, but not exclusively, to novel aminoglycoside derivatives and their use in increasing an expression of a gene having a stop codon mutation and/or in the treatment of genetic disorders.

Many human genetic disorders result from nonsense mutations, where one of the three stop codons (UAA, UAG or UGA) replaces an amino acid-coding codon, leading to premature termination of the translation and eventually to truncated inactive proteins. Currently, hundreds of such nonsense mutations are known, and several were shown to account for certain cases of fatal diseases, including, for example, cystic fibrosis (CF), Duchenne muscular dystrophy (DMD), ataxia-telangiectasia, Hurler syndrome, hemophilia A, hemophilia B, Tay-Sachs, Rett Syndrome, Usher Syndrome, Severe epidermolysis bullosa and more. For many of those diseases there is presently no effective treatment.

Some aminoglycoside compounds have been shown to have therapeutic value in the treatment of several genetic diseases because of their ability to induce ribosomes to read-through stop codon mutations, generating full-length proteins from part of the mRNA molecules.

Aminoglycosides are highly potent, broad-spectrum antibiotics commonly used for the treatment of life-threatening infections. It is accepted that the mechanism of action of aminoglycoside antibiotics, such as paromomycin (see, FIG. 1), involves interaction with the prokaryotic ribosome, and, more specifically, involves binding to the decoding A-site of the 16S ribosomal RNA, which leads to protein translation inhibition and interference with the translational fidelity.

Several achievements in bacterial ribosome structure determination, along with crystal and NMR structures of bacterial A-site oligonucleotide models, have provided useful information for understanding the decoding mechanism in prokaryote cells and understanding how aminoglycosides exert their deleterious misreading of the genetic code. These studies and others have given rise to the hypothesis that the affinity of the A-site for a non-cognate mRNA-tRNA complex is increased upon aminoglycoside binding, preventing the ribosome from efficiently discriminating between non-cognate and cognate complexes.

The enhancement of termination suppression by aminoglycosides in eukaryotes is thought to occur in a similar mechanism to the aminoglycosides' activity in prokaryotes of interfering with translational fidelity during protein synthesis, namely the binding of certain aminoglycosides to the ribosomal A-site probably induce conformational changes that stabilize near-cognate mRNA-tRNA complexes, instead of inserting the release factor. Aminoglycosides have been shown to suppress various stop codons with notably different efficiencies (UGA>UAG>UAA), and the suppression effectiveness has been found to be further dependent upon the identity of the fourth nucleotide immediately downstream from the stop codon (C>U>A≥grams) as well as the local sequence context around the stop codon.

The desired characteristics of an effective read-through drug would be oral administration and little or no effect on bacteria. Antimicrobial activity of read-through drug is undesirable as any unnecessary use of antibiotics, particularly with respect to the gastrointestinal (GI) biota, due to the adverse effects caused by upsetting the GI biota equilibrium and the emergence of resistance. In this respect, in addition to the abovementioned limitations, the majority of clinical aminoglycosides are greatly selective against bacterial ribosomes, and do not exert a significant effect on cytoplasmic ribosomes of human cells.

In an effort to circumvent the abovementioned limitations, the biopharmaceutical industry is seeking new stop codon mutations suppression drugs by screening large chemical libraries for nonsense read-through activity.

The first experiments of aminoglycoside-mediated suppression of cystic fibrosis transmembrane conductance regulator protein (CFTR) stop codon mutations demonstrated that premature stop codon mutations found in the CFTR gene could be suppressed by members of the gentamicin family and Geniticin® (G-418) (see, FIG. 1), as measured by the appearance of full-length, functional CFTR in bronchial epithelial cell lines.

Suppression experiments of intestinal tissues from CFTR−/− transgenic mice mutants carrying a human CFTR-G542X transgene showed that treatment with gentamicin, and to lesser extent tobramycin, have resulted in the appearance of human CFTR protein at the glands of treated mice. Most importantly, clinical studies using double-blind, placebo-controlled, crossover trails have shown that gentamicin can suppress stop codon mutations in affected patients, and that gentamicin treatment improved transmembrane conductance across the nasal mucosa in a group of 19 patients carrying CFTR stop codon mutations. Other genetic disorders for which the therapeutic potential of aminoglycosides was tested in in-vitro systems, cultured cell lines, or animal models include DMD, Hurler syndrome, nephrogenic diabetes insipidus, nephropathic cystinosis, retinitis pigmentosa, and ataxia-telangiectasia.

However, one of the major limitations in using aminoglycosides as pharmaceuticals is their high toxicity towards mammals, typically expressed in kidney (nephrotoxicity) and ear-associated (ototoxicity) illnesses. The origin of this toxicity is assumed to result from a combination of different factors and mechanisms such as interactions with phospholipids, inhibition of phospholipases and the formation of free radicals. Although considered selective to bacterial ribosomes, most aminoglycosides bind also to the eukaryotic A-site but with lower affinities than to the bacterial A-site. The inhibition of translation in mammalian cells is also one of the possible causes for the high toxicity of these agents. Another factor adding to their cytotoxicity is their binding to the mitochondrial ribosome at the 12S rRNA A-site, whose sequence is very close to the bacterial A-site.

Many studies have been attempted to understand and offer ways to alleviate the toxicity associated with aminoglycosides, including the use of antioxidants to reduce free radical levels, as well as the use of poly-L-aspartate and daptomycin, to reduce the ability of aminoglycosides to interact with phospholipids. The role of megalin (a multiligand endocytic receptor which is especially abundant in the kidney proximal tubules and the inner ear) in the uptake of aminoglycosides has recently been demonstrated. The administration of agonists that compete for aminoglycoside binding to megalin also resulted in a reduction in aminoglycoside uptake and toxicity. In addition, altering the administration schedule and/or the manner in which aminoglycosides are administered has been investigated as means to reduce toxicity.

Despite extensive efforts to reduce aminoglycoside toxicity, few results have matured into standard clinical practices and procedures for the administration of aminoglycosides to suppress stop codon mutations, other than changes in the administration schedule. For example, the use of sub-toxic doses of gentamicin in the clinical trials probably caused the reduced read-through efficiency obtained in the in-vivo experiments compared to the in-vitro systems. The aminoglycoside Geneticin® (also known as G-418 sulfate or simply G-418, see, FIG. 1) showed the best termination suppression activity in in-vitro translation-transcription systems, however, its use as a therapeutic agent is not possible since it is lethal even at very low concentrations. For example, the LD₅₀ of G-418 against human fibroblast cells is 0.04 mg/ml, compared to 2.5-5.0 mg/ml for gentamicin, neomycin and kanamycin.

The increased sensitivity of eukaryotic ribosomes to some aminoglycoside drugs, such as G-418 and gentamicin, is intriguing but up to date could not be rationally explained because of the lack of sufficient structural data on their interaction with eukaryotic ribosomes. Since G-418 is extremely toxic even at very low concentrations, presently gentamicin is the only aminoglycoside tested in various animal models and clinical trials. Although some studies have shown that due to their relatively lower toxicity in cultured cells, amikacin and paromomycin can represent alternatives to gentamicin for stop codon mutation suppression therapy, no clinical trials with these aminoglycosides have been reported yet.

To date, nearly all suppression experiments have been performed with clinical, commercially available aminoglycosides, however, only a limited number of aminoglycosides, including gentamicin, amikacin, and tobramycin, are in clinical use as antibiotics for internal administration in humans. Among these, tobramycin do not have stop codon mutations suppression activity, and gentamicin is the only aminoglycoside tested for stop codon mutations suppression activity in animal models and clinical trials. Recently, a set of neamine derivatives were shown to promote read-through of the SMN protein in fibroblasts derived from spinal muscular atrophy (SPA) patients; however, these compounds were originally designed as antibiotics and no conclusions were derived for further improvement of the read-through activity of these derivatives.

WO 2007/113841 and WO 2012/066546 disclose classes of paromomycin-derived aminoglycosides, designed to exhibit high premature stop codon mutations readthrough activity while exerting low cytotoxicity in mammalian cells and low antimicrobial activity, and can thus be used in the treatment of genetic diseases. This class of paromomycin-derived aminoglycosides was designed by introducing certain manipulations to the paromamine core, which lead to enhanced readthrough activity and reduced toxicity and antimicrobial activity. The manipulations were made on several positions of the paromamine core.

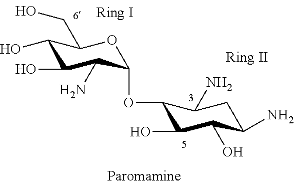

Exemplary such manipulations of the paromamine core which have been taught in these publications include a hydroxyl group at position 6′ of the aminoglycoside core; introduction of one or more monosaccharide moieties or an oligosaccharide moiety at position 3′, 5 and/or 6 of the aminoglycoside core; introduction of an (S)-4-amino-2-hydroxybutyryl (AHB) moiety at position 1 of the paromamine core; substitution of hydrogen at position 6′ by an alkyl such as a methyl substituent; and an introductions of an alkyl group at the 5″ position.

Additional background art includes Nudelman, I., et al., Bioorg Med Chem Lett, 2006. 16(24): p. 6310-5; Hobbie, S. N., et al., Nucleic Acids Res, 2007. 35(18): p. 6086-93; Kondo, J., et al., Chembiochem, 2007. 8(14): p. 1700-9; Rebibo-Sabbah, A., et al., Hum Genet, 2007. 122(3-4): p. 373-81; Azimov, R., et al., Am J Physiol Renal Physiol, 2008. 295(3): p. F633-41; Hainrichson, M., et al., Org Biomol Chem, 2008. 6(2): p. 227-39; Hobbie, S. N., et al., Proc Natl Acad Sci USA, 2008. 105(52): p. 20888-93; Hobbie, S. N., et al., Proc Natl Acad Sci USA, 2008. 105(9): p. 3244-9; Nudelman, I., et al., Adv. Synth. Catal., 2008. 350: p. 1682-1688; Nudelman, I., et al., J Med Chem, 2009. 52(9): p. 2836-45; Venkataraman, N., et al., PLoS Biol, 2009. 7(4): p. e95; Brendel, C., et al., J Mol Med (Berl), 2010. 89(4): p. 389-98; Goldmann, T., et al., Invest Ophthalmol Vis Sci, 2010. 51(12): p. 6671-80; Malik, V., et al., Ther Adv Neurol Disord, 2010. 3(6): p. 379-89; Nudelman, I., et al., Bioorg Med Chem, 2010. 18(11): p. 3735-46; Warchol, M. E., Curr Opin Otolaryngol Head Neck Surg, 2010. 18(5): p. 454-8; Lopez-Novoa, J. M., et al., Kidney Int, 2011. 79(1): p. 33-45; Rowe, S. M., et al., J Mol Med (Berl), 2011. 89(11): p. 1149-61; Vecsler, M., et al., PLoS One, 2011. 6(6): p. e20733; U.S. Pat. Nos. 3,897,412, 4,024,332, 4,029,882, and 3,996,205; Greenberg et al., J. Am. Chem. Soc., 1999, 121, 6527-6541; Kotra et al., antimicrobial agents and chemotherapy, 2000, p. 3249-3256; Haddad et al., J. Am. Chem. Soc., 2002, 124, 3229-3237; Kandasamy, J. et al., J. Med. Chem. 2012, 55, pp. 10630-10643; Duscha, S. et al., MBio, 2014, 5(5), p. e01827-14; Huth, M. E. et al., J Clin Invest., 2015, 125(2), pp. 583-92; Shulman, E. et al., J Biol Chem., 2014, 289(4), pp. 2318-30 and FR Patent No. 2,427,341, JP Patent No. 04046189. The teachings of all of these documents are incorporated by reference as if fully set forth herein.

### SUMMARY OF THE INVENTION

The present invention relates to aminoglycosides, which can be beneficially used in the treatment of genetic diseases, by exhibiting high premature stop codon mutations read-through activity, low toxicity in mammalian cells and low antimicrobial activity, as well as improved bioavailability and/or cell permeability. The presently disclosed aminoglycosides are characterized by a core structure based on Rings I, II and optionally III of paromomycin (see, FIG. 1).

According to an aspect of some embodiments of the present invention there is provided a compound represented by general formula I:

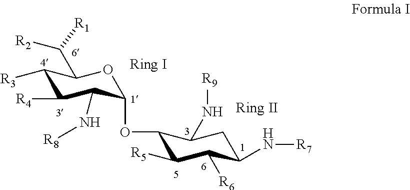

or a pharmaceutically acceptable salt thereof,

wherein:

the dashed lines indicates a stereo-configuration of position 6′ being an R configuration or an S configuration;

R₁ is selected from a hydroxy-substituted alkyl, a hydroxy-substituted alkenyl, a hydroxy-substituted cycloalkyl and a hydroxy-substituted aryl;

R₂ is selected from hydrogen, a substituted or unsubstituted alkyl and OR′, wherein R′ is selected from hydrogen, a substituted or unsubstituted alkyl, a substituted or unsubstituted cycloalkyl, a substituted or unsubstituted aryl, a substituted or unsubstituted heteroaryl, a substituted or unsubstituted alkaryl, and an acyl;

R₃-R₆ are each independently selected from hydrogen, a substituted or unsubstituted alkyl, and OR′, wherein R′ is selected from hydrogen, a monosaccharide moiety, an oligosaccharide moiety, a substituted or unsubstituted alkyl, a substituted or unsubstituted cycloalkyl, a substituted or unsubstituted aryl, a substituted or unsubstituted heteroaryl, a substituted or unsubstituted alkaryl, and an acyl; and

R₇-R₉ are each independently selected from hydrogen, acyl, an amino-substituted alpha-hydroxy acyl, a substituted or unsubstituted alkyl, a substituted or unsubstituted cycloalkyl, a substituted or unsubstituted aryl, a substituted or unsubstituted alkaryl and a cell-permealizable group.

According to some of any of the embodiments described herein for this aspect, R₁ is a hydroxy-substituted alkyl.

According to some of any of the embodiments described herein for this aspect, R₁ is hydroxymethyl.

According to some of any of the embodiments described herein for this aspect, R₂ is OR′.

According to some of any of the embodiments described herein for this aspect, R′ is hydrogen.

According to some of any of the embodiments described herein for this aspect, R₇ is selected from the hydrogen, a substituted or unsubstituted alkyl, a substituted or unsubstituted cycloalkyl, a substituted or unsubstituted alkaryl and an amino-substituted alpha-hydroxy acyl.

According to some of any of the embodiments described herein for this aspect, R₇ is selected from the hydrogen, a substituted or unsubstituted alkyl, a substituted or unsubstituted cycloalkyl, a substituted or unsubstituted alkaryl and an amino-substituted alpha-hydroxy acyl.

According to some of any of the embodiments described herein for this aspect, R₇ is alkyl or alkaryl.

According to some of any of the embodiments described herein for this aspect, R₇ is propyl or benzyl.

According to some of any of the embodiments described herein for this aspect, R₇ is an acyl.

According to some of any of the embodiments described herein for this aspect, R₇ is an amino-substituted alpha-hydroxy acyl.

According to some of any of the embodiments described herein for this aspect, each of R₃-R₆ is OR′.

According to some of any of the embodiments described herein for this aspect, each of R₃-R₆ is OR′ and R′ is hydrogen.

According to some of any of the embodiments described herein for this aspect, the compound is NB153.

According to some of any of the embodiments described herein for this aspect, the compound is NB155.

According to some of any of the embodiments described herein for this aspect, each of R₃-R₆ is OR′ and in at least one of R₃-R₆ R′ is a monosaccharide moiety or an oligosaccharide moiety.

According to some of any of the embodiments described herein for this aspect, the monosaccharide moiety is represented by Formula II:

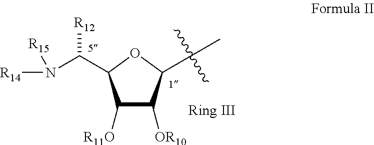

wherein the curved line denotes a position of attachment;

the dashed line indicates a stereo-configuration of position 5″ being an R configuration or an S configuration;

R₁₀ and R₁₁ are each independently selected from hydrogen, a substituted or unsubstituted alkyl, a substituted or unsubstituted cycloalkyl, a substituted or unsubstituted aryl, a substituted or unsubstituted alkaryl, and an acyl;

R₁₂ is selected from the group consisting of hydrogen, a substituted or unsubstituted alkyl, a substituted or unsubstituted cycloalkyl, and a substituted or unsubstituted aryl;

each of R₁₄ and R₁₅ is independently selected from hydrogen, acyl, an amino-substituted alpha-hydroxy acyl, a substituted or unsubstituted alkyl, a substituted or unsubstituted cycloalkyl, a substituted or unsubstituted aryl, a substituted or unsubstituted alkaryl and a cell-permealizable group, or, alternatively, R₁₄ and R₁₅ form together a heterocyclic ring.

According to some of any of the embodiments described herein for this aspect, R₅ is OR′ and R′ is the monosaccharide moiety.

According to some of any of the embodiments described herein for this aspect, the compound is represented by Formula Ia:

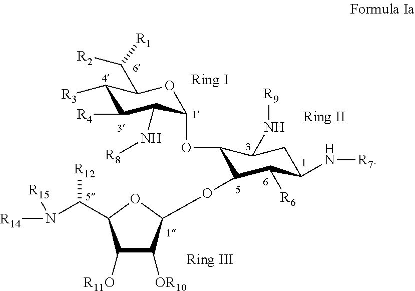

According to some of any of the embodiments described herein for this aspect, R₁₀, R₁₁, R₁₂, R₁₄ and R₁₅ are each hydrogen.

According to some of any of the embodiments described herein for this aspect, the compound is NB156.

According to some of any of the embodiments described herein for this aspect, the compound is NB157.

According to an aspect of some embodiments of the present invention there is provided a compound represented by general formula III:

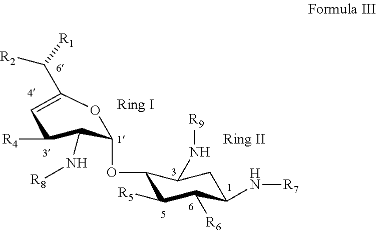

or a pharmaceutically acceptable salt thereof,

wherein:

the dashed lines indicates an optional stereo-configuration of position 6′ being an R configuration or an S configuration;

R₁ is selected from hydrogen, a substituted or unsubstituted alkyl, a substituted or unsubstituted cycloalkyl, a substituted or unsubstituted aryl;

R₂ is selected from hydrogen, a substituted or unsubstituted alkyl and OR′, wherein R′ is selected from hydrogen, a substituted or unsubstituted alkyl, a substituted or unsubstituted cycloalkyl, a substituted or unsubstituted aryl, a substituted or unsubstituted heteroaryl, a substituted or unsubstituted alkaryl, and an acyl;

R₄-R₆ are each independently selected from hydrogen, a substituted or unsubstituted alkyl, and OR′, wherein R′ is selected from hydrogen, a monosaccharide moiety, an oligosaccharide moiety, a substituted or unsubstituted alkyl, a substituted or unsubstituted cycloalkyl, a substituted or unsubstituted aryl, a substituted or unsubstituted heteroaryl, a substituted or unsubstituted alkaryl, and an acyl; and

R₇-R₉ are each independently selected from hydrogen, acyl, an amino-substituted alpha-hydroxy acyl, a substituted or unsubstituted alkyl, a substituted or unsubstituted cycloalkyl, a substituted or unsubstituted aryl, a substituted or unsubstituted alkaryl and a cell-permealizable group.

According to some of any of the embodiments described herein for this aspect, R₁ is hydrogen. Alternatively, R₁ is other than hydrogen, as described herein, and can be, for example, alkyl, cycloalkyl, aryl or hydroxyalkyl, as defined herein.

According to some of any of the embodiments described herein for this aspect, R₂ is OR′.

According to some of any of the embodiments described herein for this aspect, R′ is hydrogen.

According to some of any of the embodiments described herein for this aspect, R₇ is selected from the hydrogen, a substituted or unsubstituted alkyl, a substituted or unsubstituted cycloalkyl, a substituted or unsubstituted alkaryl and an amino-substituted alpha-hydroxy acyl.

According to some of any of the embodiments described herein for this aspect, R₇ is selected from the hydrogen, a substituted or unsubstituted alkyl, a substituted or unsubstituted cycloalkyl, a substituted or unsubstituted alkaryl and an amino-substituted alpha-hydroxy acyl.

According to some of any of the embodiments described herein for this aspect, R₇ is alkyl or alkaryl.

According to some of any of the embodiments described herein for this aspect, R₇ is propyl or benzyl.

According to some of any of the embodiments described herein for this aspect, R₇ is an acyl.

According to some of any of the embodiments described herein for this aspect, R₇ is an amino-substituted alpha-hydroxy acyl.

According to some of any of the embodiments described herein for this aspect, each of R₄-R₆ is OR′.

According to some of any of the embodiments described herein for this aspect, each of R₄-R₆ is OR′ and R′ is hydrogen.

According to some of any of the embodiments described herein for this aspect, the compound is NB154.

According to some of any of the embodiments described herein for this aspect, each of R₄-R₆ is OR′ and in at least one of R₄-R₆ R′ is a monosaccharide moiety or an oligosaccharide moiety.

According to some of any of the embodiments described herein for this aspect, the monosaccharide moiety is represented by Formula II:

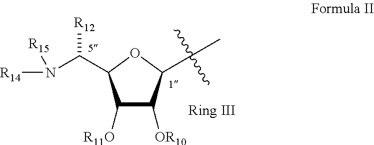

wherein the curved line denotes a position of attachment;

the dashed line indicates a stereo-configuration of position 5″ being an R configuration or an S configuration;

R₁₀ and R₁₁ are each independently selected from hydrogen, a substituted or unsubstituted alkyl, a substituted or unsubstituted cycloalkyl, a substituted or unsubstituted aryl, a substituted or unsubstituted alkaryl, and an acyl;

R₁₂ is selected from the group consisting of hydrogen, a substituted or unsubstituted alkyl, a substituted or unsubstituted cycloalkyl, and a substituted or unsubstituted aryl;

each of R₁₄ and R₁₅ is independently selected from hydrogen, acyl, an amino-substituted alpha-hydroxy acyl, a substituted or unsubstituted alkyl, a substituted or unsubstituted cycloalkyl, a substituted or unsubstituted aryl, a substituted or unsubstituted alkaryl and a cell-permealizable group, or, alternatively, R₁₄ and R₁₅ form together a heterocyclic ring.

According to some of any of the embodiments described herein for this aspect, R₅ is OR′ and R′ is the monosaccharide moiety.

According to some of any of the embodiments described herein for this aspect, the compound is represented by Formula IIIa:

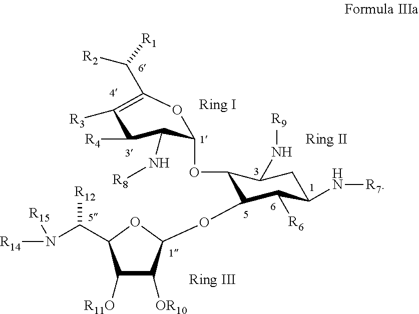

According to some of any of the embodiments described herein for this aspect, R₁₀, R₁₁, R₁₂, R₁₄ and R₁₅ are each hydrogen.

According to some of any of the embodiments described herein for this aspect, the compound is NB158.

According to some of any of the embodiments described herein for this aspect, the compound is NB159.

It is to be noted that herein throughout, the stereoconfiguration of Rings I, II and III, if present, can be any possible, compatible configuration, and are therefore not to be limited to the illustration of these rings in the general Formulae presented herein. Exemplary stereroconfigurations are presented hereinunder.

Substituents not shown in Formula II, la or IIIa at positions such as 6′, 1″, 2″, 3″, 4″ and 5″ are typically hydrogen, although other substituents, such as, but not limited, as defined for R′, are also contemplated.

According to an aspect of some embodiments of the present invention there is provided a pharmaceutical composition comprising the compound as described herein in any one of the embodiments and any combination thereof, and a pharmaceutically acceptable carrier.

According to some of any of the embodiments described herein, the pharmaceutical composition is packaged in a packaging material and identified in print, in or on the packaging material, for use in the treatment of a genetic disorder with a premature stop-codon truncation mutation and/or a protein truncation phenotype.

According to an aspect of some embodiments of the present invention there is provided a method for treating a genetic disorder with a premature stop-codon truncation mutation and/or a protein truncation phenotype, the method comprising administering to a subject in need thereof a therapeutically effective amount of the compound as described herein in any one of the embodiments and any combination thereof.

According to an aspect of some embodiments of the present invention there is provided a compound as described herein in any one of the embodiments and any combination thereof, for use in the treatment of a genetic disorder with a premature stop-codon truncation mutation and/or a protein truncation phenotype.

According to an aspect of some embodiments of the present invention there is provided a use of the compound as described herein in any one of the embodiments and any combination thereof, in the manufacture of a medicament for treating a genetic disorder with a premature stop-codon truncation mutation and/or a protein truncation phenotype.

According to some of any of the embodiments described herein, the genetic disorder is selected from the group consisting of cystic fibrosis (CF), Duchenne muscular dystrophy (DMD), ataxia-telangiectasia, Hurler syndrome, hemophilia A, hemophilia B, Usher syndrome, Tay-Sachs, Becker muscular dystrophy (BMD), Congenital muscular dystrophy (CMD), Factor VII deficiency, Familial atrial fibrillation, Hailey-Hailey disease, McArdle disease, Mucopolysaccharidosis, Nephropathic cystinosis, Polycystic kidney disease, Rett syndrome, Spinal muscular atrophy (SMA), cystinosis, Severe epidermolysis bullosa, Dravet syndrome, X-linked nephrogenic diabetes insipidus (XNDI), X-linked retinitis pigmentosa and cancer.

According to an aspect of some embodiments of the present invention there is provided a method of increasing the expression level of a gene having a premature stop-codon mutation, the method comprising translating the gene into a protein in the presence of a compound as described herein in any of the respective embodiments and any combination thereof.

According to an aspect of some embodiments of the present invention there is provided a compound as described herein in any of the respective embodiments and any combination thereof for use in increasing the expression level of a gene having a premature stop-codon mutation.

According to an aspect of some embodiments of the present invention there is provided a use of a compound as described herein in any of the respective embodiments and any combination thereof in the manufacture of a medicament for increasing the expression level of a gene having a premature stop-codon mutation.

According to some of any of the embodiments described herein, the premature stop-codon mutation has an RNA code selected from the group consisting of UGA, UAG and UAA.

According to some of any of the embodiments described herein, the protein is translated in a cytoplasmic translation system.

According to some of any of the embodiments described herein, the compound is used in a mutation suppression amount.

According to some of any of the embodiments described herein, an inhibition of translation IC₅₀ of the compound in a eukaryotic cytoplasmic translation system is greater that an inhibition of translation IC₅₀ of the compound in a ribosomal translation system.

According to some of any of the embodiments described herein, an inhibition of translation IC₅₀ of the compound in a eukaryotic cytoplasmic translation system is greater that an inhibition of translation IC₅₀ of the compound in a prokaryotic translation system.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 (Background Art) presents the chemical structures of some known families of aminoglycosides;

FIGS. 2A-B present ¹H NMR magnetic anisotropy spectra of Compound 35 (upper spectrum) and Compound 36 (lower spectrum), showing the difference in chemical shift values for the assigned protons in the NMR spectra (FIG. 2A), and the corresponding MαNP Sector Rule (FIG. 2B).

FIG. 3 presents comparative plots showing in vitro stop codon suppression levels induced by Compound 1 (-▪-), NB153 (-▴-), and NB155 (-Δ-) in R3X nonsense mutation construct representing USH1 genetic disease.

FIGS. 4A-D presents comparative plots showing in vitro stop codon suppression levels induced by NB74 (-Δ-), NB156 (-▴-), and gentamicin (--▪--) (left) and by NB124 (-Δ-), NB157 (-▴-), and gentamicin (--▪--) (right), in nonsense constructs representing R3X (USH1) (FIG. 4A), R245X (USH1) (FIG. 11B), Q70X (HS) (FIG. 4C), and G542X (CF) (FIG. 4D).

FIG. 5A presents comparative stop-codon mutation readthrough plots, showing percent readthrough as a function of concentration of WT with NB156 (readthrough to 50% renilla), comparing the readthrough of several different mutations;

FIG. 5B presents comparative stop-codon mutation readthrough plots, showing fold increase of readthrough after exposure to NB156 from non-treated control as a function of NB156 concentration, comparing the readthrough of several different mutations;

FIG. 6A presents comparative stop-codon mutation readthrough plots, showing percent readthrough as a function of concentration of WT with NB157 (readthrough to 50% renilla), comparing the readthrough of several different mutations; and

FIG. 6B presents comparative stop-codon mutation readthrough plots, showing fold increase of readthrough after exposure to NB157 from non-treated control as a function of NB157 concentration, comparing the readthrough of several different mutations.

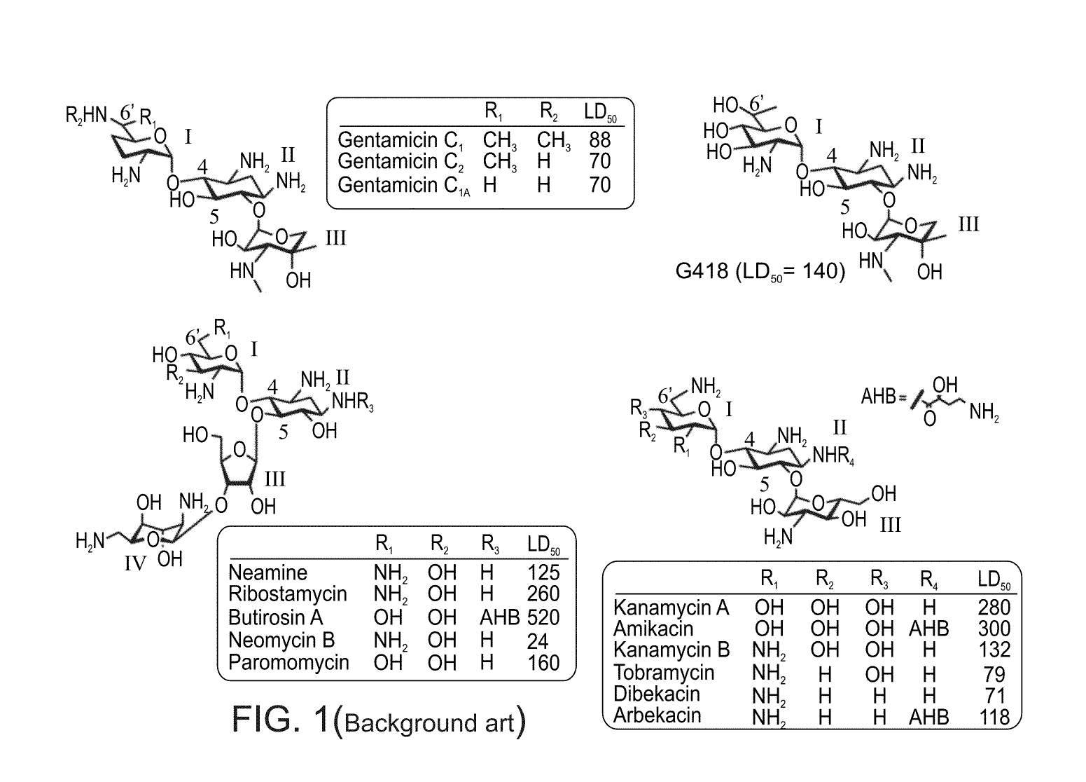

### DESCRIPTION OF SPECIFIC EMBODIMENTS OF THE INVENTION

The present invention, in some embodiments thereof, relates to aminoglycosides and more particularly, but not exclusively, to novel aminoglycoside derivatives and their use in increasing an expression of a gene having a stop codon mutation and/or in the treatment of genetic disorders.

Specifically, the present invention, in some embodiments thereof, relates to a novel aminoglycoside compounds, derived from paromomycin, which exhibit high premature stop codon mutations readthrough activity while exerting low toxicity in mammalian cells, and which are characterized by improved bioavailability and/or cell permeability. Embodiments of the present invention are further of pharmaceutical compositions containing these compounds, and of uses thereof in the treatment of genetic disorders. Embodiments of the present invention are further of processes of preparing these compounds.

The principles and operation of the present invention may be better understood with reference to the figures and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

As discussed hereinabove, the use aminoglycosides as therapeutic agents is limited primarily due to their high toxicity. In the context of treatment of genetic disorders, such a use is further limited by the antibacterial activity exhibited by the aminoglycosides, which can also translate into toxicity.

Additional limitations associated with aminoglycosides include low bioavailability, which typically requires an intravenous or subcutaneous administration, and poor permeability into eukaryotic cells, which typically requires administration of high doses which are associated with adverse side effected. It is assumed that the high water solubility and polarity of aminoglycosides limits their absorbance through intestinal tissues and their permeability through cell membranes.

As further discussed hereinabove, several structural manipulations on the structure of paromamine have given rise to synthetic aminoglycosides which have been shown to exert improved premature stop codon mutations readthrough activity while exerting low toxicity in mammalian cells. WO 2007/113841 and WO 2012/066546, which are incorporated by reference as if fully set forth herein, describe such aminoglycosides.

While further deciphering the structure-activity relationship of such aminoglycosides, in an attempt to further improve their therapeutic effect in the context of genetic disorders, the present inventor has designed numerous additional modifications, at varying positions of the paromamine structure, which are collectively represented herein by Formulae I and Ia.

While reducing the present invention to practice, exemplary novel aminoglycosides structures were prepared. As demonstrated in the Examples section that follows, these compounds were shown to exhibit high readthrough activity of disease-causing nonsense mutations as well as reduced toxicity.

The compounds presented herein ca be collectively represented by Formula I and Formula III, as described herein.

Compounds of Formula I:

According to an aspect of some embodiments of the present invention, there are provided novel aminoglycoside (AMG) compounds (also referred to herein as “aminoglycoside derivatives”), which are collectively represented by Formula I:

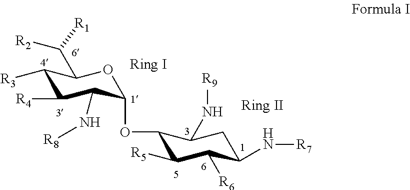

or a pharmaceutically acceptable salt thereof,

wherein:

the dashed lines indicates a stereo-configuration of position 6′ being an R configuration or an S configuration;

R₁ is selected from a hydroxy-substituted alkyl, a hydroxy-substituted alkenyl, a hydroxy-substituted cycloalkyl and a hydroxy-substituted aryl;

R₂ is selected from hydrogen, a substituted or unsubstituted alkyl and OR′, wherein R′ is selected from hydrogen, a substituted or unsubstituted alkyl, a substituted or unsubstituted cycloalkyl, a substituted or unsubstituted aryl, a substituted or unsubstituted heteroaryl, a substituted or unsubstituted alkaryl, and an acyl;

R₃-R₆ are each independently selected from hydrogen, a substituted or unsubstituted alkyl, and OR′, wherein R′ is selected from hydrogen, a monosaccharide moiety, an oligosaccharide moiety, a substituted or unsubstituted alkyl, a substituted or unsubstituted cycloalkyl, a substituted or unsubstituted aryl, a substituted or unsubstituted heteroaryl, a substituted or unsubstituted alkaryl, and an acyl; and

R₇-R₉ are each independently selected from hydrogen, acyl, an amino-substituted alpha-hydroxy acyl, a substituted or unsubstituted alkyl, a substituted or unsubstituted cycloalkyl, a substituted or unsubstituted aryl, a substituted or unsubstituted alkaryl and a cell-permealizable group.

In some of any of the embodiments described herein, the compound is a pseudo-disaccharide, having Ring I and Ring II as depicted in Formula I.

In these embodiments, none of R₃-R₆ is OR′ in which R′ is a monosaccharide or an oligosaccharide moiety.

In some of these embodiments, one or more, or all, of R₃-R₆ is OR′.

In some of these embodiments, one or more, or all, of R₃-R₆ is OR′ and R′ is hydrogen. In these embodiments, one or more, or all, of R₃-R₆ is hydroxy.

Alternatively, one or more, or all, of R₃-R₆ is OR′ and R′ in one or more, or all, of R₃-R₆ is other than hydrogen.

For example, on some embodiments, one or more, or all, of R₃-R₆ is OR′ and R′ in one or more, or all, of R₃-R₆ is independently an alkyl, which can be substituted or unsubstituted. In these embodiments, one or more, or all, of R₃-R₆ is an alkloxy, as defined herein.

For example, on some embodiments, one or more, or all, of R₃-R₆ is OR′ and R′ in one or more, or all, of R₃-R₆ is independently an aryl, which can be substituted or unsubstituted. In these embodiments, one or more, or all, of R₃-R₆ is an aryloxy, as defined herein.

In some of these embodiments, the aryl is unsubstituted such that one or more, or all of R₃-R₆, independently, can be, as non-limiting examples, phenyloxy, 1-anthryloxy, 1-naphthyloxy, 2-naphthyloxy, 2-phenanthryloxy and 9-phenanthryloxy.

In some of these embodiments, one or more of the aryls in one or more of OR′ is a substituted aryl, such that one or more, or all of R₃-R₆, independently, can be, as non-limiting examples, an aryloxy in which the aryl is 2-(N-ethylamino)phenyl, 2-(N-hexylamino)phenyl, 2-(N-methylamino)phenyl, 2,4-dimethoxyphenyl, 2-acetamidophenyl, 2-aminophenyl, 2-carboxyphenyl, 2-chlorophenyl, 2-ethoxyphenyl, 2-fluorophenyl, 2-hydroxymethylphenyl, 2-hydroxyphenyl, 2-hydroxyphenyl, 2-methoxycarbonylphenyl, 2-methoxyphenyl, 2-methylphenyl, 2-N,N-dimethylaminophenyl, 2-trifluoromethylphenyl, 3-(N,N-dibutylamino)phenyl, 3-(N,N-diethylamino)phenyl, 3,4,5-trimethoxyphenyl, 3,4-dichlorophenyl, 3,4-dimethoxyphenyl, 3,5-dimethoxyphenyl, 3-aminophenyl, 3-biphenylyl, 3-carboxyphenyl, 3-chloro-4-methoxyphenyl, 3-chlorophenyl, 3-ethoxycarbonylphenyl, 3-ethoxyphenyl, 3-fluorophenyl, 3-hydroxymethylphenyl, 3-hydroxyphenyl, 3-isoamyloxyphenyl, 3-isobutoxyphenyl, 3-isopropoxyphenyl, 3-methoxyphenyl, 3-methylphenyl, 3-N,N-dimethylaminophenyl, 3-tolyl, 3-trifluoromethylphenyl, 4-(benzyloxy)phenyl, 4-(isopropoxycarbonyl)phenyl, 4-(N,N-diethylamino)phenyl, 4-(N,N-dihexylamino)phenyl, 4-(N,N-diisopropylamino)phenyl, 4-(N,N-dimethylamino)phenyl, 4-(N,N-di-n-pentylamino)phenyl, 4-(n-hexyloxycarbonyl)phenyl, 4-(N-methylamino)phenyl, 4-(trifluoromethyl)phenyl, 4-aminophenyl, 4-benzyloxyphenyl, 4-biphenylyl, 4-butoxyphenyl, 4-butyramidophenyl, 4-carboxyphenyl, 4-chlorophenyl, 4-ethoxycarbonylphenyl, 4-hexanamidophenyl, 4-hydroxymethylphenyl, 4-hydroxyphenyl, 4-iodophenyl, 4-isobutylphenyl, 4-isobutyramidophenyl, 4-isopropoxyphenyl, 4-isopropylphenyl, 4-methoxyphenyl, 4-methylphenyl, 4-n-hexanamidophenyl, 4-n-hexyloxyphenyl, 4-n-hexylphenyl, 4-nitrophenyl, 4-nitrophenyl, 4-propionamidophenyl, 4-tolyl, 4-trifluoromethylphenyl and/or 4-valeroyloxycarbonylphenyl.

In some of these embodiments, one or more, or all, of R₃-R₆ is OR′ and R′ is independently a heteroaryl, which can be substituted or unsubstituted. In these embodiments, one or more, or all, of R₃-R₆ is a heteroaryloxy, as defined herein.

In some embodiments, one or more, or all of R₃-R₆, independently, can be, as non-limiting examples, 2-anthryloxy, 2-furyloxy, 2-indolyloxy, 2-naphthyloxy, 2-pyridyloxy, 2-pyrimidyloxy, 2-pyrryloxy, 2-quinolyloxy, 2-thienyloxy, 3-furyloxy, 3-indolyloxy, 3-thienyloxy, 4-imidazolyloxy, 4-pyridyloxy, 4-pyrimidyloxy, 4-quinolyloxy, 5-methyl-2-thienyloxy and 6-chloro-3-pyridyloxy.

In some of any of the embodiments described herein, R₃ is aryloxy or heteroaryloxy, as described herein.

In some of any of the embodiments described herein, R₃ is OR′ and R′ is a substituted or unsubstituted alkyl or alkenyl, for example, methyl, ethyl, propyl, butyl, pentyl, propenyl, 2-hydroxyethyl, 3-hydroxypropyl, 2,3-dihydroxypropyl and methoxymethyl.

In some of any of the embodiments described herein, R₃ is OR′ and R′ is hydrogen.

In some of any of the embodiments described herein, R₄ is OR′ and R′ is hydrogen.

In some of any of the embodiments described herein, R₆ is OR′ and R′ is hydrogen.

In some of any of the embodiments described herein, each of R₃ and R₄ is OR′ and R′ is hydrogen.

In some of any of the embodiments described herein, one or more of, or all, of R₃-R₆ are OR′.

In some of any of the embodiments described herein, when one or more, or all, of R₃-R₆ is OR′ and when one or more, or all, of the R′ moiety is other than hydrogen, R′ can be the same or different for each of R₃-R₆.

In some of these embodiments, when in one or more, or all, of R₃-R₆, R′ is other than hydrogen, R′ can be, for example, independently, alkyl, alkenyl, alkynyl, cycloalkyl, aryl or heteroaryl, each being optionally substituted, as described herein.

In some of any of the embodiments described herein, in one or more, or all, of R₃-R₆, R′ is independently an acyl, forming an ester (a carboxylate) at the respective position.

Herein throughout, the term “acyl” describes a —C(═O)—R″ group, wherein R″ is as described herein.

In some of any of the embodiments described herein, when R′ is an acyl, R″ is a hydrocarbon chain, as described herein, optionally substituted. In some embodiments, the hydrocarbon chain is of 2 to 18 carbon atoms in length. In some embodiments, the acyl is a hydrocarbon acyl radical having from 2 to 18 carbon atoms, optionally substituted by one or more of halo, nitro, hydroxy, amine, cyano, thiocyano, and alkoxy.

Herein, the term “hydrocarbon” or “hydrocarbon radical” describes an organic moiety that includes, as its basic skeleton, a chain of carbon atoms, also referred to herein as a backbone chain, substituted mainly by hydrogen atoms. The hydrocarbon can be saturated or non-saturated, be comprised of aliphatic, alicyclic and/or aromatic moieties, and can optionally be substituted by one or more substituents (other than hydrogen). A substituted hydrocarbon may have one or more substituents, whereby each substituent group can independently be, for example, alkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, azide, sulfonamide, carboxy, thiocarbamate, urea, thiourea, carbamate, amide, and hydrazine, and any other substituents as described herein.

The hydrocarbon moiety can optionally be interrupted by one or more heteroatoms, including, without limitation, one or more oxygen, nitrogen (substituted or unsubstituted, as defined herein for —NR′—) and/or sulfur atoms.

In some embodiments of any of the embodiments described herein relating to a hydrocarbon, the hydrocarbon is not interrupted by any heteroatom, nor does it comprise heteroatoms in its backbone chain, and can be an alkylene chain, or be comprised of alkyls, cycloalkyls, aryls, alkenes and/or alkynes, covalently attached to one another in any order.

In some of any of the embodiments described herein, when R′ is an acyl, the acyl can be derived from a carboxylic acid, such that the ester formed at the respective position is derived from, for example, a saturated or unsaturated and/or substituted or unsubstituted aliphatic carboxylic acid, including, but not limited to, acetic acid, propionic acid, butyric acid, isobutyric acid, tert-butylacetic acid, valeric acid, isovaleric acid, caproic acid, caprylic acid, decanoic acid, dodecanoic acid, lauric acid, tridecanoic acid, myristic acid, pentadecanoic acid, palmitic acid, margaric acid, stearic acid, acrylic acid, crotonic acid, undecylenic acid, oleic acid, hexynoic acid, heptynoic acid, octynoic acid; a saturated or unsaturated alicyclic carboxylic acid, including, but not limited to, cyclobutanecarboxylic acid, cyclopentanecarboxylic acid, cyclopentenecarboxylic acid, methylcyclopentenecarboxylic acid, cyclohexanecarboxylic acid, dimethylcyclohexanecarboxylic acid, dipropylcyclohexanecarboxylic acid; a saturated or unsaturated, alicyclic aliphatic carboxylic acid, including, but not limited to, cyclopentaneacetic acid, cyclopentanepropionic acid, cyclohexaneacetic acid, cyclohexanebutyric acid, methylcyclohexaneacetic acid, a substituted or unsubstituted aromatic carboxylic acid, benzoic acid, toluic acid, naphthoic acid, ethylbenzoic acid, isobutylbenzoic acid, methylbutylbenzoic acid; an aromatic carboxylic acid, including, but not limited to, phenylacetic acid, benzoic acid, phenylpropionic acid, phenylvaleric acid, cinnamic acid, phenylpropiolic acid, naphthylacetic acid; a halo-alkoxyhydrocarbon carboxylic acid; a nitro-alkoxyhydrocarbon carboxylic acid; a hydroxy-alkoxyhydrocarbon carboxylic acid; an amino-alkoxyhydrocarbon carboxylic acid; a cyano-alkoxyhydrocarbon carboxylic acid; a thiocyano-alkoxyhydrocarbon carboxylic acid; as well as mono-acetic acid; di-acetic acid, trichloroacetic acid; 1,2,3,4,5,6-hexachlorocyclohexanecarboxylic acid, 1,2-dibromo-4-methylcyclohexanecarboxylic acid, 1,6-dibromo-3-methylcyclohexanecarboxylic acid, 1-bromo-3,5-dimethylcyclohexanecarboxylic acid, 2-chlorocyclohexanecarboxylic acid, 4-chlorocyclohexanecarboxylic acid, 2,3-dibromo-2-methylcyclohexanecarboxylic acid, 2,4,6-trinitrobenzoic acid, 2,5-dibromo-2-methylcyclohexanecarboxylic acid, 2-bromo-4-methylcyclohexanecarboxylic acid, 2-nitro-1-methyl-cyclobutanecarboxylic acid, 3,4-dinitrobenzoic acid, 3,5-dinitrobenzoic acid, 3-bromo-2,2,3-trimethylcyclopentanecarboxylic acid, 3-bromo-2-methylcyclohexanecarboxylic acid, 3-bromo-3-methylcyclohexanecarboxylic acid, 4-bromo-2-methylcyclohexanecarboxylic acid, 5-bromo-2-methylcyclohexanecarboxylic acid, ′4,4-dichlorobenzilic acid, 4,5-dibromo-2-methylcyclohexanecarboxylic acid, 5-bromo-2-methylcyclohexanecarboxylic acid, 6-bromo-2-methylcyclohexanecarboxylic acid, 5,6-dibromo-2-methylcyclohexanecarboxylic acid, 6-bromo-3-methylcyclohexanecarboxylic acid, anisic acid, cyanoacetic acid, cyanopropionic acid, ethoxyformic acid (ethyl hydrogen carbonate), gallic acid, homogentisic acid, o-, m-, and p-chlorobenzoic acid, lactic acid, mevalonic acid, o-, m-, p-nitrobenzoic acid, p-hydroxybenzoic acid, salicylic acid, shikimic acid, thiocyanoacetic acid, trimethoxybenzoic acid, trimethoxycinnamic acid, veratric acid, α- and β-chloropropionic acid, α- and γ-bromobutyric acid and α- and δ-iodovaleric acid, β-resorcylic acid.

In some of any of the embodiments described herein, when one or more of R₇-R₉ is acyl, the acyl is such that R′ is an alkyl or alkaryl or aryl, each of which being optionally substituted by one or more amine substituents.

In some of any of the embodiments described herein, when one or more of R₇-R₉ is acyl, the acyl is such that R′ is an alkyl or alkaryl or aryl, each of which being optionally substituted by one or more amine substituents.

In some of these embodiments, R′ in the acyl is a substituted alkyl, and in some embodiments, R′ is substituted by hydroxy at the a position with respect to the carbonyl group, such that the acyl is α-hydroxy-acyl.

In some embodiments, the α-hydroxy-acyl is further substituted by one or more amine groups, and is an amino-substituted α-hydroxy-acyl.

In some of the embodiments of an acyl group as described herein, the amine substituents can be, for example, at one or more of positions β, γ, δ, and/or ω of the moiety R′, with respect to the acyl.

Exemplary amino-substituted α-hydroxy-acyls include, without limitation, the moiety (S)-4-amino-2-hydroxybutyryl, which is also referred to herein as AHB. According to some embodiments of the present invention, an alternative to the AHB moiety can be the α-hydroxy-β-aminopropionyl (AHP) moiety. Additional exemplary amino-substituted α-hydroxy-acyls include, but are not limited to, L-(−)-γ-amino-α-hydroxybutyryl, L(−)-δ-amino-α-hydroxyvaleryl, L-(−)-β-benzyloxycarbonylamino-α-hydroxypropionyl, a L-(−)-δ-benzyloxycarbonylamino-α-hydroxyvaleryl.

It is noted herein that according to some embodiments of the present invention, other moieties which involve a combination of carbonyl(s), hydroxyl(s) and amino group(s) along a lower alkyl exhibiting any stereochemistry, are contemplated as optional substituents in place of AHB and/or AHP, including, for example, 2-amino-3-hydroxybutanoyl, 3-amino-2-hydroxypentanoyl, 5-amino-3-hydroxyhexanoyl and the likes.

In some of any of the embodiments described herein, one or more of R₃-R₆ is other than OR′. In some of any of the embodiments described herein, one or more of R₃-R₆ is hydrogen.

In some of any of the embodiments described herein R₃ is hydrogen.

In some of any of the embodiments described herein R₄ is hydrogen.

In some of any of the embodiments described herein R₃ and R₄ are each hydrogen.

In some of any of the embodiments described herein, one or more of R₃-R₆ is OR′ and R′ is independently a monosaccharide moiety or an oligosaccharide moiety, as defined herein, such that the compound is a pseudo-trisaccharide, a pseudo-tetrasaccharide, a pseudo-pentasaccharide, a pseudo hexasaccharide, etc.

Whenever one or more of R₃-R₆ is OR′ and R′ is a monosaccharide moiety or an oligosaccharide moiety and one or more of R₃-R₆ is not OR′ in which R′ is a monosaccharide moiety or an oligosaccharide moiety, the one or more of R₃-R₆ which is not OR′ in which R′ is a monosaccharide moiety or an oligosaccharide moiety can be as described herein for any of the respective embodiments for R₃-R₆.

The term “monosaccharide”, as used herein and is well known in the art, refers to a simple form of a sugar that consists of a single saccharide molecule which cannot be further decomposed by hydrolysis. Most common examples of monosaccharides include glucose (dextrose), fructose, galactose, and ribose. Monosaccharides can be classified according to the number of carbon atoms of the carbohydrate, i.e., triose, having 3 carbon atoms such as glyceraldehyde and dihydroxyacetone; tetrose, having 4 carbon atoms such as erythrose, threose and erythrulose; pentose, having 5 carbon atoms such as arabinose, lyxose, ribose, xylose, ribulose and xylulose; hexose, having 6 carbon atoms such as allose, altrose, galactose, glucose, gulose, idose, mannose, talose, fructose, psicose, sorbose and tagatose; heptose, having 7 carbon atoms such as mannoheptulose, sedoheptulose; octose, having 8 carbon atoms such as 2-keto-3-deoxy-manno-octonate; nonose, having 9 carbon atoms such as sialose; and decose, having 10 carbon atoms. Monosaccharides are the building blocks of oligosaccharides like sucrose (common sugar) and other polysaccharides (such as cellulose and starch).

The term “oligosaccharide” as used herein refers to a compound that comprises two or more monosaccharide units, as these are defined herein, linked to one another via a glycosyl bond (—O—). Preferably, the oligosaccharide comprises 2-6 monosaccharides, more preferably the oligosaccharide comprises 2-4 monosaccharides and most preferably the oligosaccharide is a disaccharide moiety, having two monosaccharide units.

In some of any of the embodiments described herein, the monosaccharide is a pentose moiety, such as, for example, represented by Formula II. Alternatively, the monosaccharide moiety is hexose. Further alternatively, the monosaccharide moiety is other than pentose or hexose, for example, a hexose moiety as described in U.S. Pat. No. 3,897,412.

In some of any of the embodiments described herein, the monosaccharide moiety is a ribose, represented by Formula II:

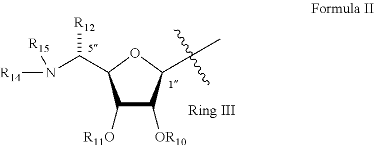

wherein the curved line denotes a position of attachment;

the dashed line indicates a stereo-configuration of position 5″ being an R configuration or an S configuration;

R₁₀ and R₁₁ are each independently selected from hydrogen, a substituted or unsubstituted alkyl, a substituted or unsubstituted cycloalkyl, a substituted or unsubstituted aryl, a substituted or unsubstituted alkaryl, and an acyl;

R₁₂ is selected from the group consisting of hydrogen, a substituted or unsubstituted alkyl, a substituted or unsubstituted cycloalkyl, and a substituted or unsubstituted aryl;

each of R₁₄ and R₁₅ is independently selected from hydrogen, acyl, an amino-substituted alpha-hydroxy acyl, a substituted or unsubstituted alkyl, a substituted or unsubstituted cycloalkyl, a substituted or unsubstituted aryl, a substituted or unsubstituted alkaryl and a cell-permealizable group, or, alternatively, R₁₄ and R₁₅ form together a heterocyclic ring.

In some of any of the embodiments described herein, R₁₂ is hydrogen.

In some of any of the embodiments described herein, R₁₂ is other than hydrogen. In some of these embodiments, R₁₂ is alkyl, cycloalkyl or aryl, and in some embodiments, R₁₂ is alkyl, preferably a lower alkyl, for example, methyl. The alkly, cycloakly or aryl can be substituted, as defined herein, or unsubstituted, preferably unsubstituted.

In some of any of the embodiments where one or more of R₃-R₆ is OR′ and R′ is a monosaccharide moiety or an oligosaccharide moiety, one or more of the hydroxy groups in the monosaccharide or oligosaccharide moiety/moieties are substituted by an acyl, forming an ester (a carboxylate), as described herein in any of the respective embodiments.

In some of these embodiments, one or both of R₁₀ and R₁₁ is an acyl, forming an ester at the respective position(s), as described herein.

In some of any of the embodiments described herein, one of R₃-R₆ is OR′ and R′ is a monosaccharide moiety such that the compound is a pseudo-trisaccharide.

In some of any of the embodiments described herein for a pseudo-trisaccharide, one or more, or all, of R₁₀ and R₁₁, can be an acyl, as described herein.

In some of any of the embodiments described herein for a pseudo-trisaccharide, one or more, or all, of R₃-R₆ are OR′, such that in one of R₃-R₆, R′ is a monosaccharide moiety, and in the others, R′ is as defined herein (e.g., hydrogen).

In some of any of the embodiments described herein, R₅ is OR′ in which R′ is a monosaccharide moiety.

In some of these embodiments, the compound is represented by Formula Ia:

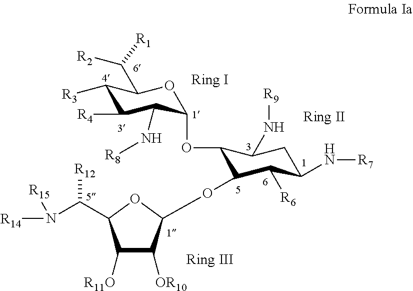

with the variables being as described herein for Formulae I and II, including any combination thereof.

In some of any of the embodiments described herein for Formulae I and Ia, R₂ is hydrogen.

In some of any of the embodiments described herein for Formulae I and Ia, R₂ is hydrogen, R₂ is OR′, and R′ is hydrogen.

In some of any of the embodiments described herein for Formulae I and Ia, R₂ is hydrogen, R₂ is OR′, and R′ is other than hydrogen.

In some of any of the embodiments described herein for Formulae I and Ia, R₂ is hydrogen, R₂ is OR′ and R′ is an acyl, forming as ester at this position, as described herein.

In some embodiments, R₂ is OR′ and R′ is an alkyl, preferably selected from the group consisting of methyl, ethyl and propyl.

In some of any of the embodiments described herein, R₂ is alkyl, and in some of these embodiments R₂ is a substituted alkyl, for example, an alkyl substituted by one or more amine groups (aminoalkyl).

In some of any of the embodiments described herein, R₂ is a substituted or unsubstituted alkyl, as defined herein, or a substituted or unsubstituted cycloalkyl, as defined herein.

In some of any of the embodiments described herein, R₂ is a substituted or unsubstituted aryl, as defined herein.

R₁, according to any of embodiments of Formulae I and Ia herein, comprises a hydroxy substituent, which forms a “diol-like” structure on Ring I when R₂ is OR′ and R′ is hydrogen.

In some embodiments, the hydroxy substituents is configured such that a “diol-like” structure as described herein is capable of interacting with a respective gene at a desired site, as is discussed in further detail hereinafter and in the art.

For example, in some embodiments, R₁ is configured such that a hydroxy substituent is 1-6, or 1-4, or 1-3, or 2, carbon atoms away from the hydroxy OR′ group of R₂.

Thus, in some embodiments, R₁ is a hydroxy-substituted alkyl, a hydroxy-substituted alkenyl, a hydroxy-substituted cycloalkyl or a hydroxy-substituted aryl, in which the hydroxy substituent is at a position of the alkyl, alkenyl, cycloalkyl or aryl, that allows the above-described interaction.

In some embodiments, R₁ is a hydroxy-substituted alkyl, or a hydroxy-substituted alkenyl, and the hydroxy substituent is a terminal substituent.

In some of these embodiments, a R₁ is a hydroxy-substituted alkyl is also referred to herein as hydroxy alkyl.

In some of any of the embodiments of R₁ in Formula I or Ia, the alkyl or alkenyl are 1 to 10, or 1 to 8, preferably 1 to 6, or 1 to 4.

In some of any of the embodiments of R₁ in Formula I or Ia, R₁ is a hydroxyalkyl, wherein the alkyl can be further substituted or not.

In some of any of the embodiments described herein, R₁ is a hydroxymethyl.

In some of any of the embodiments of R₁ in Formula I or Ia, the hydroxy-substituted alkyl, alkenyl, cycloalkyl or aryl can be further substituted or not, and can, for example, include 2 or more hydroxy groups.

According to some of any of the embodiments of Formula I or Ia, one or both of the amine substituents at positions 1 (R₇), 3 (R₉), 2′ (R₈) or 5″ (R₁₄ and/or R₁₅, if present) of the aminoglycoside structure is modified, such that one or more of R₇-R₉ and of R₁₄ and R₁₅, if present, is not hydrogen.

Herein throughout, an amine which bears a substituent other than hydrogen is referred to herein as a “modified amine substituent” or simply as a “modified amine”.

According to some embodiments of the present invention, one or both of the amine substituents at positions 1 (R₇), 3 (R₉), 2′ (R₈) or 5″ (R₁₄ and/or R₁₅, if present) of the aminoglycoside structure is modified to include a hydrophobic moiety such as alkyl, cycloalkyl, alkaryl and/or aryl, or a group which is positively-charged at physiological pH and which can increase cell permeability of the compound (also referred to herein interchangeably as “cell-permealizable group” or “cell-permealizing group”), such as guanine or guanidine groups, as defined herein, or, alternatively, hydrazine, hidrazide, thiohydrazide, urea and thiourea.

In some of any of the embodiments described herein, and particularly for pseudo-disaccharide compounds, the amine substituent at position 1 (Ring II) in Formula I, is a modified amine, as described herein, such that R₇ is other than hydrogen. Alternatively, or in addition, one or more of R₈ and R₉ is other than hydrogen.

In some of these embodiments, one or more of R₇-R₉ and of R₁₄ and R₁₅, if present, is independently an alkyl, a cell-permealizable group, as described herein, or an acyl, such as, for example, an alpha-hydroxy acyl or an amino-substituted alpha-hydroxy acyl, as described herein.

Exemplary moieties represented by one or more of R₇-R₉ and of R₁₄ and R₁₅, if present, include, but are not limited to, hydrogen, (R/S)-4-amino-2-hydroxybutyryl (AHB), (R/S)-3-amino-2-hydroxypropionyl (AHP), 5-aminopentanoyl, 5-hydroxypentanoyl, formyl, —C(═O)—O-methyl, —C(═O)—O-ethyl, —C(═O)—O-benzyl, -β-amino-α-hydroxypropionyl, -δ-amino-α-hydroxyvaleryl, -β-benzyloxycarbonylamino-α-hydroxypropionyl, -δ-benzyloxycarbonylamino-α-hydroxyvaleryl, methylsulfonyl, phenylsulfonyl, benzoyl, propyl, isopropyl, —(CH₂)₂NH₂, —(CH₂)₃NH₂, —CH₂CH(NH₂)CH₃, —(CH₂)₄NH₂, —(CH₂)₅NH₂, —(C H₂)₂NH-ethyl, —(CH₂)₂NH(CH₂)₂NH₂, —(CH₂)₃NH(CH₂)₃NH₂, —(CH₂)₃NH(CH₂)₄NH(CH₂)₃NH₂, —CH(—NH₂)CH₂(OH), —CH(—OH)CH₂(NH₂), —CH(—OH)—(CH₂)₂(NH₂), —CH(—NH₂)—(CH₂)₂(OH), —CH(-CH₂NH₂)—(CH₂OH), —(CH₂)₄NH(CH₂)₃NH₂, —(CH₂)₂NH(CH₂)₂N H(CH₂)₂NH₂, —(CH₂)₂N(CH₂CH₂NH₂)₂, —CH₂—C(═O)NH₂, —CH(CH₃)—C(═O)NH₂, —CH₂-phenyl, —CH(i-propyl)-C(═O)NH₂, —CH(benzyl)-C(═O)NH₂, —(CH₂)₂OH, —(CH₂)₃OH and —CH(CH₂OH)₂.

In some of any of the embodiments described herein, R₇ is hydrogen, (R/S)-4-amino-2-hydroxybutyryl (AHB), (R/S)-3-amino-2-hydroxypropionyl, 5-aminopentanoyl, 5-hydroxypentanoyl, formyl, —C(═O)—O-methyl, —C(═O)—O-ethyl, —C(═O)—O-benzyl, -β-amino-α-hydroxypropionyl, -δ-amino-α-hydroxyvaleryl, -β-benzyloxycarbonylamino-α-hydroxypropionyl, -δ-benzyloxycarbonylamino-α-hydroxyvaleryl, methylsulfonyl, phenylsulfonyl, benzoyl, propyl, isopropyl, —(CH₂)₂NH₂, —(CH₂)₃NH₂, —CH₂CH(NH₂)CH₃, —(CH₂)₄NH₂, —(CH₂)₅NH₂, —(C H₂)₂NH-ethyl, —(CH₂)₂NH(CH₂)₂NH₂, —(CH₂)₃NH(CH₂)₃NH₂, —(CH₂)₃NH(CH₂)₄NH(CH₂)₃NH₂, —CH(-NH₂)CH₂(OH), —CH(—OH)CH₂(NH₂), —CH(—OH)—(CH₂)₂(NH₂), —CH(-N H₂)—(CH₂)₂(OH), —CH(-CH₂NH₂)—(CH₂OH), —(CH₂)₄NH(CH₂)₃NH₂, —(CH₂)₂NH(CH₂)₂N H(CH₂)₂NH₂, —(CH₂)₂N(CH₂CH₂NH₂)₂, —CH₂—C(═O)NH₂, —CH(CH₃)—C(═O)NH₂, —CH₂-phenyl, —CH(i-propyl)-C(═O)NH₂, —CH(benzyl)-C(═O)NH₂, —(CH₂)₂OH, —(CH₂)₃OH or —CH(CH₂OH)₂.

In some of any of the embodiments described herein, one or both of R₈ and R₉ is independently hydrogen, (R/S)-4-amino-2-hydroxybutyryl (AHB), (R/S)-3-amino-2-hydroxypropionate (AHP), (R/S)-3-amino-2-hydroxypropionyl, 5-aminopentanoyl, 5-hydroxypentanoyl, formyl, —COO-methyl, —COO-ethyl, —COO-benzyl, -β-amino-α-hydroxypropionyl, -δ-amino-α-hydroxyvaleryl, -β-benzyloxycarbonylamino-α-hydroxypropionyl, -δ-benzyloxycarbonylamino-α-hydroxyvaleryl, methylsulfonyl, phenylsulfonyl, benzoyl, propyl, isopropyl, —(CH₂)₂NH₂, —(CH₂)₃NH₂, —CH₂CH(NH₂)CH₃, —(CH₂)₄NH₂, —(CH₂)₅NH₂, —(C H₂)₂NH-ethyl, —(CH₂)₂NH(CH₂)₂NH₂, —(CH₂)₃NH(CH₂)₃NH₂, —(CH₂)₃NH(CH₂)₄NH(CH₂)₃NH₂, —CH(-NH₂)CH₂(OH), —CH(—OH)CH₂(NH₂), —CH(—OH)—(CH₂)₂(NH₂), —CH(-NH₂)—(CH₂)₂(OH), —CH(-CH₂NH₂)—(CH₂OH), —(CH₂)₄NH(CH₂)₃NH₂, —(CH₂)₂NH(CH₂)₂NH (CH₂)₂NH₂, —(CH₂)₂N(CH₂CH₂NH₂)₂, —CH₂—C(═O)NH₂, —CH(CH₃)—C(═O)NH₂, —CH₂-phenyl, —CH(i-propyl)-C(═O)NH₂, —CH(benzyl)-C(═O)NH₂, —(CH₂)₂OH, —(CH₂)₃OH or —CH(CH₂OH)₂.

In some of any of the embodiments described herein, an amino-substituted alpha-hydroxy acyl is (S)-4-amino-2-hydroxybutyryl (AHB).

In some of any of the embodiments described herein, one or more R₇-R₉ and R₁₄ and R₁₅, if present, is a cell-permealizable group as defined herein, and in some embodiments, it is a guanidyl, as defined herein.

In some of any of the embodiments described herein, one or more R₇-R₉ and R₁₄ and R₁₅, if present, is a hydrophobic moiety such as alkyl, cycloalkyl, alkaryl and/or aryl.

In some of any of the embodiments described herein, one or more R₇-R₉ and R₁₄ and R₁₅, if present, is an acyl, as defined herein for the respective embodiments, and in some of these embodiments, the acyl can independently be an amino-substituted alpha-hydroxy acyl, as defined herein.

In some of the embodiments where one or more R₇-R₉ and R₁₄ and R₁₅, if present, is alkyl, the alkyl can be, for example, a lower alkyl, of 1-4 carbon atoms, such as, but not limited to, methyl, ethyl, propyl, butyl, isopropyl, and isobutyl, each being optionally substituted, as described herein.

In some of these embodiments, the alkyl is independently a non-substituted alkyl, such as, but not limited to, ethyl, propyl and isopropyl.

In some of these embodiments, the alkyl is independently a substituted methyl, such as, but not limited to, an alkaryl such as benzyl.

Alternatively, one or more R₇-R₉ and R₁₄ and R₁₅, if present, is independently a cycloalkyl, and the cycloalkyl can be, for example, cyclopropyl, cyclobutyl, cyclopentyl and cyclohexyl.

Further alternatively, one or more R₇-R₉ and R₁₄ and R₁₅, if present, is independently an aryl, and the aryl can be, for example, a substituted or unsubstituted phenyl. Non-limiting examples include unsubstituted phenyl and toluene.

In some of any of the embodiments described herein, the amine substituent at position 1 (R₇, Ring II) in Formula Ia or Ib, is a modified amine, as described herein, such that R₇ is other than hydrogen.

In some of these embodiments, R₇ can be alkyl, cycloalkyl, alkaryl, aryl, an acyl, or an amino-substituted α-hydroxy acyl, as defined herein, such as, for example, (S)-4-amino-2-hydroxybutyryl (AHB), or (S)-4-amino-2-hydroxypropionyl (AHP).

In some of the embodiments where R₇ is alkyl, the alkyl can be, for example, a lower alkyl, of 1-4 carbon atoms, such as, but not limited to, methyl, ethyl, propyl, butyl, isopropyl, and isobutyl, each being optionally substituted, as described herein.

In some of these embodiments, the alkyl is independently a non-substituted alkyl, such as, but not limited to, ethyl, propyl and isopropyl.

In some of these embodiments, the alkyl is independently a substituted methyl, such as, but not limited to, an alkaryl such as benzyl.

Alternatively, R₇ is cycloalkyl, and the cycloalkyl can be, for example, cyclopropyl, cyclobutyl, cyclopentyl and cyclohexyl.

Further alternatively, R₇ is aryl, and the aryl can be, for example, a substituted or unsubstituted phenyl. Non-limiting examples include unsubstituted phenyl and toluene.

In some of any of the embodiments described herein, R₇ is alkyl, cycloalkyl or aryl, as described herein.

In some of any of the embodiments described herein, R₇ is a cell-permealizable group, as defined herein, and in some embodiments, R₇ is guanidinyl.

In some of any of the embodiments of Formula Ia, one or both of R₁₄ and R₁₅ is other than hydrogen, such that an amine at position 5″ is a modified amine, as defined herein. In some of these embodiments, one or both of R₁₄ and R₁₅ is a cell-permealizable group such as, for example, a guanidine group. Alternatively, one or both of R₁₄ and R₁₅ is alkyl, cycloalkyl or aryl, as defined, for example, for any of the embodiments of R₇.

In some of any of the embodiments described herein throughout, whenever a variable is defined as an unsubstituted aryl, the unsubstituted aryl can be, for example, phenyl, 1-anthryl, 1-naphthyl, 2-naphthyl, 2-phenanthryl and/or 9-phenanthryl.

In some of any of the embodiments described herein, whenever a variable is defined as a substituted or unsubstituted heteroaryl, the heteroaryl can be, for example, 2-anthryl, 2-furyl, 2-indolyl, 2-naphthyl, 2-pyridyl, 2-pyrimidyl, 2-pyrryl, 2-quinolyl, 2-thienyl, 3-furyl, 3-indolyl, 3-thienyl, 4-imidazolyl, 4-pyridyl, 4-pyrimidyl, 4-quinolyl, 5-methyl-2-thienyl and/or 6-chloro-3-pyridyl.

In some of any of the embodiments described herein, whenever a variable is defined as a substituted aryl, the aryl can be, for example, 2-(N-ethylamino)phenyl, 2-(N-hexylamino)phenyl, 2-(N-methylamino)phenyl, 2,4-dimethoxyphenyl, 2-acetamidophenyl, 2-aminophenyl, 2-carboxyphenyl, 2-chlorophenyl, 2-ethoxyphenyl, 2-fluorophenyl, 2-hydroxymethylphenyl, 2-hydroxyphenyl, 2-hydroxyphenyl, 2-methoxycarbonylphenyl, 2-methoxyphenyl, 2-methylphenyl, 2-N,N-dimethylaminophenyl, 2-trifluoromethylphenyl, 3-(N,N-dibutylamino)phenyl, 3-(N,N-diethylamino)phenyl, 3,4,5-trimethoxyphenyl, 3,4-dichlorophenyl, 3,4-dimethoxyphenyl, 3,5-dimethoxyphenyl, 3-aminophenyl, 3-biphenylyl, 3-carboxyphenyl, 3-chloro-4-methoxyphenyl, 3-chlorophenyl, 3-ethoxycarbonylphenyl, 3-ethoxyphenyl, 3-fluorophenyl, 3-hydroxymethylphenyl, 3-hydroxyphenyl, 3-isoamyloxyphenyl, 3-isobutoxyphenyl, 3-isopropoxyphenyl, 3-methoxyphenyl, 3-methylphenyl, 3-N,N-dimethylaminophenyl, 3-tolyl, 3-trifluoromethylphenyl, 4-(benzyloxy)phenyl, 4-(isopropoxycarbonyl)phenyl, 4-(N,N-diethylamino)phenyl, 4-(N,N-dihexylamino)phenyl, 4-(N,N-diisopropylamino)phenyl, 4-(N,N-dimethylamino)phenyl, 4-(N,N-di-n-pentylamino)phenyl, 4-(n-hexyloxycarbonyl)phenyl, 4-(N-methylamino)phenyl, 4-(trifluoromethyl)phenyl, 4-aminophenyl, 4-benzyloxyphenyl, 4-biphenylyl, 4-butoxyphenyl, 4-butyramidophenyl, 4-carboxyphenyl, 4-chlorophenyl, 4-ethoxycarbonylphenyl, 4-hexanamidophenyl, 4-hydroxymethylphenyl, 4-hydroxyphenyl, 4-iodophenyl, 4-isobutylphenyl, 4-isobutyramidophenyl, 4-isopropoxyphenyl, 4-isopropylphenyl, 4-methoxyphenyl, 4-methylphenyl, 4-n-hexanamidophenyl, 4-n-hexyloxyphenyl, 4-n-hexylphenyl, 4-nitrophenyl, 4-nitrophenyl, 4-propionamidophenyl, 4-tolyl, 4-trifluoromethylphenyl and/or 4-valeroyloxycarbonylphenyl.

In some of any of the embodiments described herein for Formula I or Ia, R₁ is a hydroxyalkyl, for example, hydroxymethyl, and R₂ is OR′, wherein R′ is hydrogen.

In some of any of the embodiments described herein for Formula I, R₁ is a hydroxyalkyl, for example, hydroxymethyl, and each of R₃-R₆ is OR′, wherein R′ is hydrogen.

In some of any of the embodiments described herein for Formula I, R₁ is a hydroxyalkyl, for example, hydroxymethyl, and each of R₂-R₆ is OR′, wherein R′ is hydrogen.

In some of these embodiments, each of R₇-R₉ is hydrogen.

Exemplary such compounds are referred to herein as NB153 and NB155, as presented in Table 1 hereinbelow.

In some of any of the embodiments described herein for Formula Ia, R₁ is a hydroxyalkyl, for example, hydroxymethyl, and each of R₃, R₄ and R₆ is OR′, wherein R′ is hydrogen.

In some of any of the embodiments described herein for Formula Ia, R₁ is a hydroxyalkyl, for example, hydroxymethyl, and each of R₂, R₃, R₄ and R₆ is OR′, wherein R′ is hydrogen.

In some of these embodiments, each of R₇-R₉ is hydrogen.

In some of these embodiments, each of R₁₀ and R₁₁ is hydrogen.

In some of these embodiments, R₁₂ is hydrogen. Alternatively, R₁₂ is alkyl such as methyl.

In some of these embodiments, R₁₄ and R₁₅ are each hydrogen.

Exemplary such compounds are referred to herein as NB156 and NB157, as presented in Table 1 hereinbelow.

Compounds of Formula III:

According to an aspect of some embodiments of the present invention, there are provided novel aminoglycoside (AMG) compounds (also referred to herein as “aminoglycoside derivatives”), which are collectively represented by Formula III:

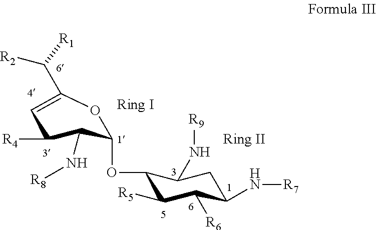

wherein:

the dashed line indicates an optional stereo-configuration of position 6′ being an R configuration or an S configuration;

R₁ is selected from hydrogen, a substituted or unsubstituted alkyl, a substituted or unsubstituted cycloalkyl, a substituted or unsubstituted aryl;

R₂ is selected from hydrogen, a substituted or unsubstituted alkyl and OR′, wherein R′ is selected from hydrogen, a substituted or unsubstituted alkyl, a substituted or unsubstituted cycloalkyl, a substituted or unsubstituted aryl, a substituted or unsubstituted heteroaryl, a substituted or unsubstituted alkaryl, and an acyl;

R₄-R₆ are each independently selected from hydrogen, a substituted or unsubstituted alkyl, and OR′, wherein R′ is selected from hydrogen, a monosaccharide moiety, an oligosaccharide moiety, a substituted or unsubstituted alkyl, a substituted or unsubstituted cycloalkyl, a substituted or unsubstituted aryl, a substituted or unsubstituted heteroaryl, a substituted or unsubstituted alkaryl, and an acyl; and

R₇-R₉ are each independently selected from hydrogen, acyl, an amino-substituted alpha-hydroxy acyl, a substituted or unsubstituted alkyl, a substituted or unsubstituted cycloalkyl, a substituted or unsubstituted aryl, a substituted or unsubstituted alkaryl and a cell-permealizable group.

Compounds represented by Formula III are also referred to herein as “unsaturated Glucosamine (Ring I)-containing” compound.

According to some of any of the embodiments described herein, R₇-R₉ are as described herein for Formula I or Ia, in any of the respective embodiments and any combination thereof.

According to some of any of the embodiments described herein, R₄-R₆ are as described herein for Formula I or Ia, in any of the respective embodiments and any combination thereof (e.g., for R₃-R₆ in Formula I).

According to some of any of the embodiments described herein, one or more of R₄-R₆ is a monosaccharide moiety or an oligosaccharide moiety, as described herein for Formula I, in any of the respective embodiments and any combination thereof.

According to some of any of the embodiments described herein, one or more of R₅ is a monosaccharide moiety or an oligosaccharide moiety, as described herein for Formula I, in any of the respective embodiments and any combination thereof.

In some of these embodiments, the monosaccharide moiety is represented by Formula II, as described herein in the context of embodiments of Formula I or Ia.

According to some of any of the embodiments described herein, one or more of R₄-R₆ is a monosaccharide moiety or an oligosaccharide moiety, as described herein for Formula I, in any of the respective embodiments and any combination thereof.

In some of these embodiments, the compound is represented by Formula IIIa:

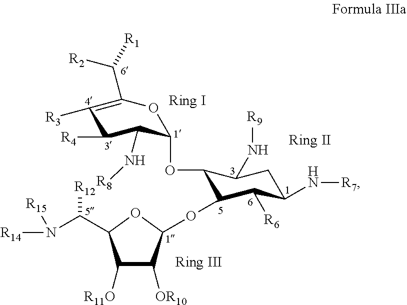

wherein each of the variables is independently as defined herein in any of the respective embodiments and any combination thereof.

In some of any of the embodiments of Formula IIIa described herein, R₁₀, R₁₁, R₁₂, R₁₄ and R₁₅ are as described herein for Formula II or Ia, in any of the respective embodiments and any combination thereof.

In some of any of the embodiments of Formulae III and IIIa described herein, R₁₀, R₁₁, R₁₂, R₁₄ and R₁₅ are each hydrogen.

In some of any of the embodiments of Formulae III and IIIa described herein, R₂ is as described herein for Formula I or Ia.

In some of any of the embodiments described herein for Formulae III and IIIa, R₁ is hydrogen.

In some of any of the embodiments described herein for Formulae III and IIIa, R₁ is other than hydrogen.

In some of any of the embodiments described herein for Formulae III and IIIa, R₁ is alkyl, and in some embodiments it is a lower alkyl, of 1 to 4 carbon atoms, including, but not limited to, methyl, ethyl, propyl, butyl, isopropyl, and isobutyl.

In some of any of the embodiments described herein for Formulae III and IIIa, R₁ is a non-substituted alkyl.

In some of any of the embodiments described herein for Formulae III and IIIa, R₁ is methyl.

Alternatively, in some of any of the embodiments described herein for Formulae III and IIIa, R₁ is cycloalkyl, including, but not limited to, cyclopropyl, cyclobutyl, cyclopentyl and cyclohexyl.

Further alternatively, in some of any of the embodiments described herein for Formulae III and IIIa, R₁ is aryl, such as substituted or unsubstituted phenyl. Non-limiting examples include unsubstituted phenyl and toluene.

Further alternatively, in some of any of the embodiments described herein for Formulae III and IIIa, R₁ is alkaryl, such as, for example, a substituted or unsubstituted benzyl.

In some of any of the embodiments described herein for Formulae III and IIIa, R₁ is alkyl, alkenyl or alkynyl, each being substituted or unsubstituted.

In some of any of the embodiments described herein for Formulae III and IIIa, R₁ is alkyl, preferably a lower alkyl, for example, methyl, ethyl, propyl, butyl or pentyl.

In some of any of the embodiments described herein for Formulae III and IIIa, R₁ is or comprises an aryl which can be substituted or unsubstituted. In some embodiments of Formulae III and IIIa, R₁ is an unsubstituted aryl and can be, as non-limiting examples, phenyl, 1-anthryl, 1-naphthyl, 2-naphthyl, 2-phenanthryl or 9-phenanthryl.

In some embodiments of Formulae III and IIIa, R₁ is a substituted aryl, and can be, as non-limiting examples, 2-(N-ethylamino)phenyl, 2-(N-hexylamino)phenyl, 2-(N-methylamino)phenyl, 2,4-dimethoxyphenyl, 2-acetamidophenyl, 2-aminophenyl, 2-carboxyphenyl, 2-chlorophenyl, 2-ethoxyphenyl, 2-fluorophenyl, 2-hydroxymethylphenyl, 2-hydroxyphenyl, 2-hydroxyphenyl, 2-methoxycarbonylphenyl, 2-methoxyphenyl, 2-methylphenyl, 2-N,N-dimethylaminophenyl, 2-trifluoromethylphenyl, 3-(N,N-dibutylamino)phenyl, 3-(N,N-diethylamino)phenyl, 3,4,5-trimethoxyphenyl, 3,4-dichlorophenyl, 3,4-dimethoxyphenyl, 3,5-dimethoxyphenyl, 3-aminophenyl, 3-biphenylyl, 3-carboxyphenyl, 3-chloro-4-methoxyphenyl, 3-chlorophenyl, 3-ethoxycarbonylphenyl, 3-ethoxyphenyl, 3-fluorophenyl, 3-hydroxymethylphenyl, 3-hydroxyphenyl, 3-isoamyloxyphenyl, 3-isobutoxyphenyl, 3-isopropoxyphenyl, 3-methoxyphenyl, 3-methylphenyl, 3-N,N-dimethylaminophenyl, 3-tolyl, 3-trifluoromethylphenyl, 4-(benzyloxy)phenyl, 4-(isopropoxycarbonyl)phenyl, 4-(N,N-diethylamino)phenyl, 4-(N,N-dihexylamino)phenyl, 4-(N,N-diisopropylamino)phenyl, 4-(N,N-dimethylamino)phenyl, 4-(N,N-di-n-pentylamino)phenyl, 4-(n-hexyloxycarbonyl)phenyl, 4-(N-methylamino)phenyl, 4-(trifluoromethyl)phenyl, 4-aminophenyl, 4-benzyloxyphenyl, 4-biphenylyl, 4-butoxyphenyl, 4-butyramidophenyl, 4-carboxyphenyl, 4-chlorophenyl, 4-ethoxycarbonylphenyl, 4-hexanamidophenyl, 4-hydroxymethylphenyl, 4-hydroxyphenyl, 4-iodophenyl, 4-isobutylphenyl, 4-isobutyramidophenyl, 4-isopropoxyphenyl, 4-isopropylphenyl, 4-methoxyphenyl, 4-methylphenyl, 4-n-hexanamidophenyl, 4-n-hexyloxyphenyl, 4-n-hexylphenyl, 4-nitrophenyl, 4-nitrophenyl, 4-propionamidophenyl, 4-tolyl, 4-trifluoromethylphenyl or 4-valeroyloxycarbonylphenyl.

In some of any of the embodiments described herein for Formulae III and IIIa, R₁ is or comprises a substituted or unsubstituted heteroaryl, and can be, as non-limiting examples, 2-anthryl, 2-furyl, 2-indolyl, 2-naphthyl, 2-pyridyl, 2-pyrimidyl, 2-pyrryl, 2-quinolyl, 2-thienyl, 3-furyl, 3-indolyl, 3-thienyl, 4-imidazolyl, 4-pyridyl, 4-pyrimidyl, 4-quinolyl, 5-methyl-2-thienyl and 6-chloro-3-pyridyl.

In some of any of the embodiments described herein for Formulae III and IIIa, R₁ is or comprises an amine, as defined herein, and can be, as non-limiting examples, —NH₂, —NHCH₃, —N(CH₃)₂, —NH—CH₂—CH₂—NH₂, —NH—CH₂—CH₂—OH and —NH—CH₂—CH(OCH₃)₂. In some of any of the embodiments described herein for Formula III or IIIa, R₁ is a hydroalkyl, for example, hydroxymethyl.

In some of any of the embodiments described herein for Formula III or IIIa, R₂ is OR′, wherein R′ is hydrogen.

In some of any of the embodiments described herein for Formula I, R₁ is hydrogen, and each of R₃-R₆ is OR′, wherein R′ is hydrogen.

In some of any of the embodiments described herein for Formula I, R₁ is a hydrogen, and each of R₂-R₆ is OR′, wherein R′ is hydrogen.

In some of these embodiments, each of R₇-R₉ is hydrogen.

An exemplary such compound is referred to herein as NB154, as presented in the Examples section that follows.

In some of any of the embodiments described herein for Formula IIIa, R₁ is hydrogen, and each of R₃, R₄ and R₆ is OR′, wherein R′ is hydrogen.

In some of any of the embodiments described herein for Formula Ia, R₁ is hydrogen, and each of R₂, R₃, R₄ and R₆ is OR′, wherein R′ is hydrogen.

In some of these embodiments, each of R₇-R₉ is hydrogen.

In some of these embodiments, each of R₁₀ and R₁₁ is hydrogen.

In some of these embodiments, R₁₂ is hydrogen. Alternatively, R₁₂ is alkyl such as methyl.

In some of these embodiments, R₁₄ and R₁₅ are each hydrogen.

Exemplary such compounds are referred to herein as NB158 and NB159, as presented in the Examples section that follows.

According to some of any of the embodiments of the present invention, for Formula I, Ia, III and IIIa, excluded from the scope of the present invention are compounds known in the art, including any of the documents cited in the Background section of the instant application, which are encompassed by Formula I, Ia, III or IIIa.

Exemplary compounds which are excluded from the scope of the present embodiments include, but are not limited to, gentamicin, geneticin, fortimycin, apramycin, arbekacin, dibekacin, geneticin (G-418, G418), habekacin, kanamycin, Lividomycin, paromomycin, streptomycin and tobramycin.

Table A below present chemical structures of some exemplary aminoglycosides previously described in the cited art.

TABLE A

| Number | Structure |
|---|---|
| n/a | 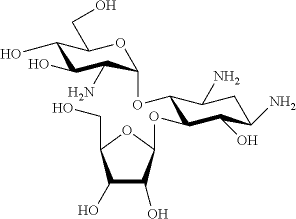 |
| NB30 | 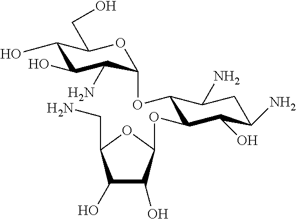 |
| n/a | 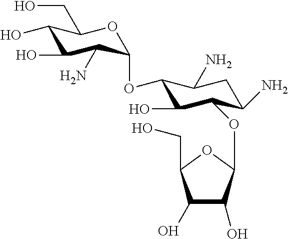 |
| n/a | 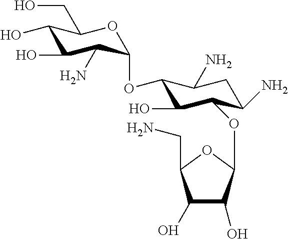 |
| n/a | 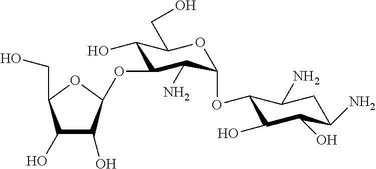 |
| n/a | 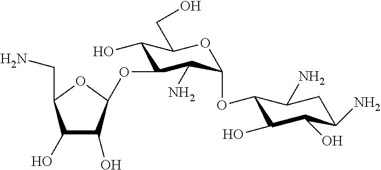 |
| n/a | 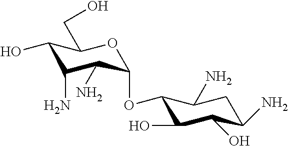 |
| n/a | 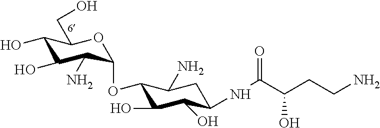 |
| n/a | 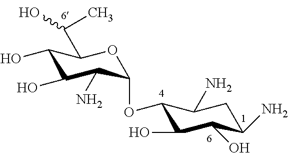 |
| n/a | 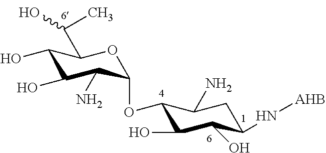 |
| NB54 | 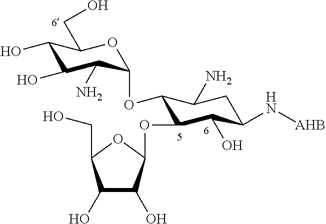 |
| NB74 | 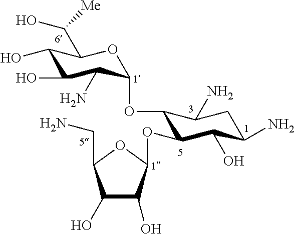 |
| NB84 | 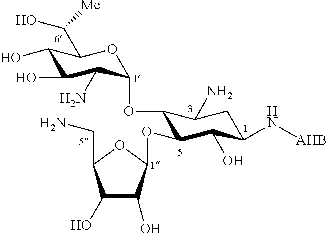 |
| NB118 | 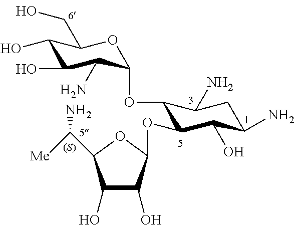 |
| NB119 | 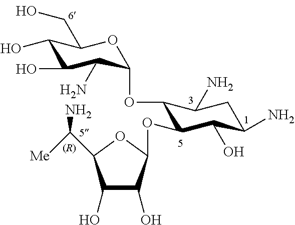 |
| NB122 | 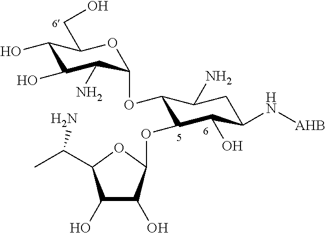 |
| NB123 | 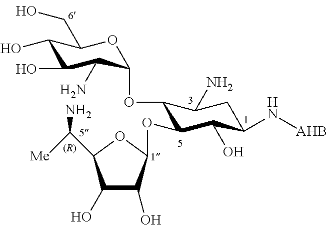 |
| NB124 | 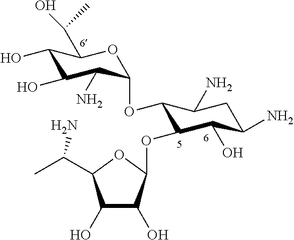 |
| NB125 | 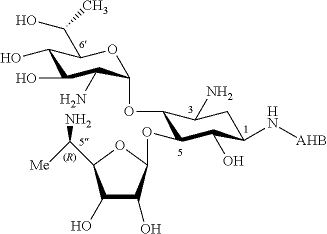 |
| NB127 | 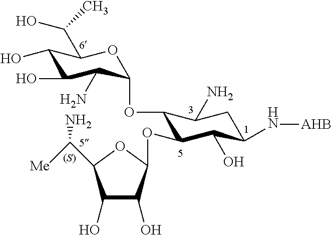 |
| NB128 | 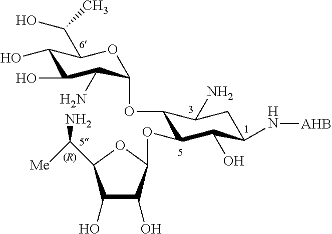 |

For any of the embodiments described herein, and any combination thereof, the compound may be in a form of a salt, for example, a pharmaceutically acceptable salt.

As used herein, the phrase “pharmaceutically acceptable salt” refers to a charged species of the parent compound and its counter-ion, which is typically used to modify the solubility characteristics of the parent compound and/or to reduce any significant irritation to an organism by the parent compound, while not abrogating the biological activity and properties of the administered compound. A pharmaceutically acceptable salt of a compound as described herein can alternatively be formed during the synthesis of the compound, e.g., in the course of isolating the compound from a reaction mixture or re-crystallizing the compound.

In the context of some of the present embodiments, a pharmaceutically acceptable salt of the compounds described herein may optionally be an acid addition salt comprising at least one basic (e.g., amine and/or guanidine) group of the compound which is in a positively charged form (e.g., wherein the basic group is protonated), in combination with at least one counter-ion, derived from the selected base, that forms a pharmaceutically acceptable salt.

The acid addition salts of the compounds described herein may therefore be complexes formed between one or more basic groups of the compound and one or more equivalents of an acid.

Depending on the stoichiometric proportions between the charged group(s) in the compound and the counter-ion in the salt, the acid additions salts can be either mono-addition salts or poly-addition salts.

The phrase “mono-addition salt”, as used herein, refers to a salt in which the stoichiometric ratio between the counter-ion and charged form of the compound is 1:1, such that the addition salt includes one molar equivalent of the counter-ion per one molar equivalent of the compound.

The phrase “poly-addition salt”, as used herein, refers to a salt in which the stoichiometric ratio between the counter-ion and the charged form of the compound is greater than 1:1 and is, for example, 2:1, 3:1, 4:1 and so on, such that the addition salt includes two or more molar equivalents of the counter-ion per one molar equivalent of the compound.

An example, without limitation, of a pharmaceutically acceptable salt would be an ammonium cation or guanidinium cation and an acid addition salt thereof.

The acid addition salts may include a variety of organic and inorganic acids, such as, but not limited to, hydrochloric acid which affords a hydrochloric acid addition salt, hydrobromic acid which affords a hydrobromic acid addition salt, acetic acid which affords an acetic acid addition salt, ascorbic acid which affords an ascorbic acid addition salt, benzenesulfonic acid which affords a besylate addition salt, camphorsulfonic acid which affords a camphorsulfonic acid addition salt, citric acid which affords a citric acid addition salt, maleic acid which affords a maleic acid addition salt, malic acid which affords a malic acid addition salt, methanesulfonic acid which affords a methanesulfonic acid (mesylate) addition salt, naphthalenesulfonic acid which affords a naphthalenesulfonic acid addition salt, oxalic acid which affords an oxalic acid addition salt, phosphoric acid which affords a phosphoric acid addition salt, toluenesulfonic acid which affords a p-toluenesulfonic acid addition salt, succinic acid which affords a succinic acid addition salt, sulfuric acid which affords a sulfuric acid addition salt, tartaric acid which affords a tartaric acid addition salt and trifluoroacetic acid which affords a trifluoroacetic acid addition salt. Each of these acid addition salts can be either a mono-addition salt or a poly-addition salt, as these terms are defined herein.

The present embodiments further encompass any enantiomers, diastereomers, prodrugs, solvates, hydrates and/or pharmaceutically acceptable salts of the compounds described herein.

As used herein, the term “enantiomer” refers to a stereoisomer of a compound that is superposable with respect to its counterpart only by a complete inversion/reflection (mirror image) of each other. Enantiomers are the to have “handedness” since they refer to each other like the right and left hand. Enantiomers have identical chemical and physical properties except when present in an environment which by itself has handedness, such as all living systems. In the context of the present embodiments, a compound may exhibit one or more chiral centers, each of which exhibiting an R- or an S-configuration and any combination, and compounds according to some embodiments of the present invention, can have any their chiral centers exhibit an R- or an S-configuration.

The term “diastereomers”, as used herein, refers to stereoisomers that are not enantiomers to one another. Diastereomerism occurs when two or more stereoisomers of a compound have different configurations at one or more, but not all of the equivalent (related) stereocenters and are not mirror images of each other. When two diastereoisomers differ from each other at only one stereocenter they are epimers. Each stereo-center (chiral center) gives rise to two different configurations and thus to two different stereoisomers. In the context of the present invention, embodiments of the present invention encompass compounds with multiple chiral centers that occur in any combination of stereo-configuration, namely any diastereomer.

According to some of any of the embodiments described herein, a stereo-configuration of each of position 6′ and position 5″ (if present and chiral) is independently an R configuration or an S configuration.

According to some of any of the embodiments described herein, a stereo-configuration of position 6′ is an R configuration.

According to some of any of the embodiments described herein, a stereo-configuration of position 5″, if present and chiral, is an S configuration.

According to some of any of the embodiments described herein, a stereo-configuration of position 6′ is an R configuration and a stereo-configuration of position 5″, if present and chiral, is an R configuration or an S configuration.

According to some of any of the embodiments described herein, a stereo-configuration of position 6′ is an R configuration and a stereo-configuration of position 5″, if present and chiral, is an S configuration.

The term “prodrug” refers to an agent, which is converted into the active compound (the active parent drug) in vivo. Prodrugs are typically useful for facilitating the administration of the parent drug. They may, for instance, be bioavailable by oral administration whereas the parent drug is not. A prodrug may also have improved solubility as compared with the parent drug in pharmaceutical compositions. Prodrugs are also often used to achieve a sustained release of the active compound in vivo. An example, without limitation, of a prodrug would be a compound of the present invention, having one or more carboxylic acid moieties, which is administered as an ester (the “prodrug”). Such a prodrug is hydrolyzed in vivo, to thereby provide the free compound (the parent drug). The selected ester may affect both the solubility characteristics and the hydrolysis rate of the prodrug.

The term “solvate” refers to a complex of variable stoichiometry (e.g., di-, tri-, tetra-, penta-, hexa-, and so on), which is formed by a solute (the compound of the present invention) and a solvent, whereby the solvent does not interfere with the biological activity of the solute. Suitable solvents include, for example, ethanol, acetic acid and the like.

The term “hydrate” refers to a solvate, as defined hereinabove, where the solvent is water.

The terms “hydroxyl” or “hydroxy”, as used herein, refer to an —OH group.

As used herein, the term “amine” describes a —NR′R″ group where each of R′ and R″ is independently hydrogen, alkyl, alkenyl, alkynyl, cycloalkyl, heteroalicyclic, aryl, heteroaryl, alkaryl, alkheteroaryl, or acyl, as these terms are defined herein. Alternatively, one or both of R′ and R″ can be, for example, hydroxy, alkoxy, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, carbonyl, C-carboxylate, O-carboxylate, N-thiocarbamate, O-thiocarbamate, urea, thiourea, N-carbamate, O-carbamate, C-amide, N-amide, guanyl, guanidine and hydrazine.

As used herein, the term “alkyl” describes an aliphatic hydrocarbon including straight chain and branched chain groups. The alkyl may have 1 to 20 carbon atoms, or 1-10 carbon atoms, and may be branched or unbranched. According to some embodiments of the present invention, the alkyl is a low (or lower) alkyl, having 1-4 carbon atoms (namely, methyl, ethyl, propyl and butyl).

Whenever a numerical range; e.g., “1-10”, is stated herein, it implies that the group, in this case the alkyl group, may contain 1 carbon atom, 2 carbon atoms, 3 carbon atoms, etc., up to and including 10 carbon atoms. In some embodiments, the alkyl is a lower alkyl, including 1-6 or 1-4 carbon atoms.

An alkyl can be substituted or unsubstituted. When substituted, the substituent can be, for example, one or more of an alkyl (forming a branched alkyl), an alkenyl, an alkynyl, a cycloalkyl, an aryl, a heteroaryl, a heteroalicyclic, a halo, a trihaloalkyl, a hydroxy, an alkoxy and a hydroxyalkyl as these terms are defined hereinbelow. An alkyl substituted by aryl is also referred to herein as “alkaryl”, an example of which is benzyl.

Whenever “alkyl” is described, it can be replaced also by alkenyl or alkynyl. The term “alkyl” as used herein, also encompasses saturated or unsaturated hydrocarbon, hence this term further encompasses alkenyl and alkynyl.

The term “alkenyl” describes an unsaturated alkyl, as defined herein, having at least two carbon atoms and at least one carbon-carbon double bond, e.g., allyl, vinyl, 3-butenyl, 2-butenyl, 2-hexenyl and i-propenyl. The alkenyl may be substituted or unsubstituted by one or more substituents, as described hereinabove.

The term “alkynyl”, as defined herein, is an unsaturated alkyl having at least two carbon atoms and at least one carbon-carbon triple bond. The alkynyl may be substituted or unsubstituted by one or more substituents, as described hereinabove.

The term “cycloalkyl” refers to an all-carbon monocyclic or fused ring (i.e., rings which share an adjacent pair of carbon atoms), branched or unbranched group containing 3 or more carbon atoms where one or more of the rings does not have a completely conjugated pi-electron system, and may further be substituted or unsubstituted.

Exemplary cycloalkyl groups include, for example, cyclopropyl, cyclobutyl, cyclopentyl, cyclohexyl, or cyclododecyl. The cycloalkyl can be substituted or unsubstituted. When substituted, the substituent can be, for example, one or more of an alkyl, an alkenyl, an alkynyl, a cycloalkyl, an aryl, a heteroaryl, a heteroalicyclic, a halo, a trihaloalkyl, a hydroxy, an alkoxy and a hydroxyalkyl as these terms are defined hereinbelow.

The term “aryl” describes an all-carbon monocyclic or fused-ring polycyclic (i.e., rings which share adjacent pairs of carbon atoms) groups having a completely conjugated pi-electron system. The aryl group may be unsubstituted or substituted by one or more substituents. When substituted, the substituent can be, for example, one or more of an alkyl, an alkenyl, an alkynyl, a cycloalkyl, an aryl, a heteroaryl, a heteroalicyclic, a halo, a trihaloalkyl, a hydroxy, an alkoxy and a hydroxyalkyl as these terms are defined hereinbelow.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. Representative examples are thiadiazole, pyridine, pyrrole, oxazole, indole, purine and the like. The heteroaryl group may be unsubstituted or substituted by one or more substituents. When substituted, the substituent can be, for example, one or more of an alkyl, an alkenyl, an alkynyl, a cycloalkyl, an aryl, a heteroaryl, a heteroalicyclic, a halo, a trihaloalkyl, a hydroxy, an alkoxy and a hydroxyalkyl as these terms are defined hereinbelow.

The term “heteroalicyclic”, as used herein, describes a monocyclic or fused ring group having in the ring(s) one or more atoms such as nitrogen, oxygen and sulfur. The rings may also have one or more double bonds. However, the rings do not have a completely conjugated pi-electron system. Representative examples are morpholine, piperidine, piperazine, tetrahydrofurane, tetrahydropyrane and the like. The heteroalicyclic may be substituted or unsubstituted. When substituted, the substituent can be, for example, one or more of an alkyl, an alkenyl, an alkynyl, a cycloalkyl, an aryl, a heteroaryl, a heteroalicyclic, a halo, a trihaloalkyl, a hydroxy, an alkoxy and a hydroxyalkyl as these terms are defined hereinbelow.

The term “halide”, as used herein, refers to the anion of a halo atom, i.e. F⁻, Cl⁻, Br⁻ and I⁻.

The term “halo” refers to F, Cl, Br and I atoms as substituents.

The term “alkoxide” refers to an R′—O⁻ anion, wherein R′ is as defined hereinabove.

The term “alkoxy” refers to an —OR′ group, wherein R′ is alkyl or cycloalkyl, as defined herein.

The term “aryloxy” refers to an —OR′ group, wherein R′ is aryl, as defined herein.

The term “heteroaryloxy” refers to an —OR′ group, wherein R′ is heteroaryl, as defined herein.

The term “thioalkoxy” refers to an —SR′ group, wherein R′ is alkyl or cycloalkyl, as defined herein.

The term “thioaryloxy” refers to an —SR′ group, wherein R′ is aryl, as defined herein.

The term “thioheteroaryloxy” refers to an —SR′ group, wherein R′ is heteroaryl, as defined herein.

The term “hydroxyalkyl,” as used herein, refers to an alkyl group, as defined herein, substituted with one or more hydroxy group(s), e.g., hydroxymethyl, 2-hydroxyethyl and 4-hydroxypentyl.

The term “aminoalkyl,” as used herein, refers to an alkyl group, as defined herein, substituted with one or more amino group(s).

The term “alkoxyalkyl,” as used herein, refers to an alkyl group substituted with one or more alkoxy group(s), e.g., methoxymethyl, 2-methoxyethyl, 4-ethoxybutyl, n-propoxyethyl and t-butylethyl.

The term “trihaloalkyl” refers to —CX₃, wherein X is halo, as defined herein. An exemplary haloalkyl is CF₃.

A “guanidino” or “guanidine” or “guanidinyl” or “guanidyl” group refers to an —RaNC(═NRd)-NRbRc group, where each of Ra, Rb, Re and Rd can each be as defined herein for R′ and R″.

A “guanyl” or “guanine” group refers to an RaRbNC(═NRd)- group, where Ra, Rb and Rd are each as defined herein for R′ and R″.

In some of any of the embodiments described herein, the guanidine group is —NH—C(═NH)—NH₂.

In some of any of the embodiments described herein, the guanyl group is H₂N—C(═NH)— group.

Any one of the amine (including modified amine), guanidine and guanine groups described herein is presented as a free base form thereof, but is meant to encompass an ionized form thereof at physiological pH, and/or within a salt thereof, e.g., a pharmaceutically acceptable salt thereof, as described herein.

Whenever an alkyl, cycloalkyl, aryl, alkaryl, heteroaryl, heteroalicyclic, acyl and any other moiety as described herein is substituted, it includes one or more substituents, each can independently be, but are not limited to, hydroxy, alkoxy, thiohydroxy, thioalkoxy, aryloxy, thioaryloxy, alkaryl, alkenyl, alkynyl, sulfonate, sulfoxide, thiosulfate, sulfate, sulfite, thiosulfite, phosphonate, cyano, nitro, azo, sulfonamide, carbonyl, thiocarbonyl, C-carboxylate, O-carboxylate, N-thiocarbamate, O-thiocarbamate, oxo, thiooxo, oxime, acyl, acyl halide, azo, azide, urea, thiourea, N-carbamate, O-carbamate, C-amide, N-amide, guanyl, guanidyl, hydrazine and hydrazide, as these terms are defined herein.

The term “cyano” describes a —C≡N group.

The term “nitro” describes an —NO₂ group.

The term “sulfate” describes a —O-S(═O)₂—OR′ end group, as this term is defined hereinabove, or an —O-S(═O)₂—O— linking group, as these phrases are defined hereinabove, where R′ is as defined hereinabove.

The term “thiosulfate” describes a —O—S(═S)(═O)—OR′ end group or a —O-S(═S)(═O)—O— linking group, as these phrases are defined hereinabove, where R′ is as defined hereinabove.

The term “sulfite” describes an —O—S(═O)—O—R′ end group or a —O—S(═O)—O-group linking group, as these phrases are defined hereinabove, where R′ is as defined hereinabove.

The term “thiosulfite” describes a —O—S(═S)—O—R′ end group or an —O—S(═S)—O— group linking group, as these phrases are defined hereinabove, where R′ is as defined hereinabove.

The term “sulfinate” describes a —S(═O)—OR′ end group or an —S(═O)—O-group linking group, as these phrases are defined hereinabove, where R′ is as defined hereinabove.

The term “sulfoxide” or “sulfinyl” describes a —S(═O)R′ end group or an —S(═O)— linking group, as these phrases are defined hereinabove, where R′ is as defined hereinabove.

The term “sulfonate” or “sulfonyl” describes a —S(═O)₂—R′ end group or an —S(═O)₂— linking group, as these phrases are defined hereinabove, where R′ is as defined herein.

The term “S-sulfonamide” describes a —S(═O)₂—NR′R″ end group or a —S(═O)₂—NR′— linking group, as these phrases are defined hereinabove, with R′ and R″ as defined herein.

The term “N-sulfonamide” describes an R′S(═O)₂—NR″— end group or a —S(═O)₂—NR′— linking group, as these phrases are defined hereinabove, where R′ and R″ are as defined herein.

The term “carbonyl” or “carbonate” as used herein, describes a —C(═O)—R′ end group or a —C(═O)— linking group, as these phrases are defined hereinabove, with R′ as defined herein.

The term “thiocarbonyl” as used herein, describes a —C(═S)—R′ end group or a —C(═S)— linking group, as these phrases are defined hereinabove, with R′ as defined herein.

The term “oxo” as used herein, describes a (═O) group, wherein an oxygen atom is linked by a double bond to the atom (e.g., carbon atom) at the indicated position.

The term “thiooxo” as used herein, describes a (═S) group, wherein a sulfur atom is linked by a double bond to the atom (e.g., carbon atom) at the indicated position.

The term “oxime” describes a ═N-OH end group or a ═N-O— linking group, as these phrases are defined hereinabove.

The term “acyl halide” describes a —(C═O)R″″ group wherein R″″ is halide, as defined hereinabove.

The term “azo” or “diazo” describes an —N═NR′ end group or an —N═N— linking group, as these phrases are defined hereinabove, with R′ as defined hereinabove.

The term “azide” describes an —N₃ end group.

The term “carboxylate” as used herein encompasses C-carboxylate and O-carboxylate.

The term “C-carboxylate” describes a —C(═O)—OR′ end group or a —C(═O)—O— linking group, as these phrases are defined hereinabove, where R′ is as defined herein.

The term “O-carboxylate” describes a —OC(═O)R′ end group or a —OC(═O)— linking group, as these phrases are defined hereinabove, where R′ is as defined herein.

A carboxylate can be linear or cyclic. When cyclic, R′ and the carbon atom are linked together to form a ring, in C-carboxylate, and this group is also referred to as lactone. Alternatively, R′ and O are linked together to form a ring in O-carboxylate. Cyclic carboxylates can function as a linking group, for example, when an atom in the formed ring is linked to another group.

The term “thiocarboxylate” as used herein encompasses C-thiocarboxylate and 0-thiocarboxylate.

The term “C-thiocarboxylate” describes a —C(═S)—OR′ end group or a —C(═S)—O-linking group, as these phrases are defined hereinabove, where R′ is as defined herein.

The term “O-thiocarboxylate” describes a —OC(═S)R′ end group or a —OC(═S)— linking group, as these phrases are defined hereinabove, where R′ is as defined herein.

A thiocarboxylate can be linear or cyclic. When cyclic, R′ and the carbon atom are linked together to form a ring, in C-thiocarboxylate, and this group is also referred to as thiolactone. Alternatively, R′ and O are linked together to form a ring in O-thiocarboxylate. Cyclic thiocarboxylates can function as a linking group, for example, when an atom in the formed ring is linked to another group.

The term “carbamate” as used herein encompasses N-carbamate and 0-carbamate.

The term “N-carbamate” describes an R″OC(═O)—NR′-end group or a —OC(═O)—NR′— linking group, as these phrases are defined hereinabove, with R′ and R″ as defined herein.

The term “O-carbamate” describes an —OC(═O)—NR′R″ end group or an —OC(═O)—NR′— linking group, as these phrases are defined hereinabove, with R′ and R″ as defined herein.

A carbamate can be linear or cyclic. When cyclic, R′ and the carbon atom are linked together to form a ring, in O-carbamate. Alternatively, R′ and O are linked together to form a ring in N-carbamate. Cyclic carbamates can function as a linking group, for example, when an atom in the formed ring is linked to another group.

The term “carbamate” as used herein encompasses N-carbamate and O-carbamate.

The term “thiocarbamate” as used herein encompasses N-thiocarbamate and O-thiocarbamate.

The term “O-thiocarbamate” describes a —OC(═S)—NR′R″ end group or a —OC(═S)—NR′-linking group, as these phrases are defined hereinabove, with R′ and R″ as defined herein.

The term “N-thiocarbamate” describes an R″OC(═S)NR′-end group or a —OC(═S)NR′-linking group, as these phrases are defined hereinabove, with R′ and R″ as defined herein.

Thiocarbamates can be linear or cyclic, as described herein for carbamates.

The term “dithiocarbamate” as used herein encompasses S-dithiocarbamate and N-dithiocarbamate.

The term “S-dithiocarbamate” describes a —SC(═S)—NR′R″ end group or a —SC(═S)NR′-linking group, as these phrases are defined hereinabove, with R′ and R″ as defined herein.

The term “N-dithiocarbamate” describes an R″SC(═S)NR′-end group or a —SC(═S)NR′-linking group, as these phrases are defined hereinabove, with R′ and R″ as defined herein.

The term “urea”, which is also referred to herein as “ureido”, describes a —NR′C(═O)—NR″R″′ end group or a —NR′C(═O)—NR″-linking group, as these phrases are defined hereinabove, where R′ and R″ are as defined herein and R″′ is as defined herein for R′ and R″.

The term “thiourea”, which is also referred to herein as “thioureido”, describes a —NR′—C(═S)—NR″R″′ end group or a —NR′—C(═S)—NR″— linking group, with R′, R″ and R″′ as defined herein.

The term “amide” as used herein encompasses C-amide and N-amide.

The term “C-amide” describes a —C(═O)—NR′R″ end group or a —C(═O)—NR′— linking group, as these phrases are defined hereinabove, where R′ and R″ are as defined herein.

The term “N-amide” describes a R′C(═O)—NR″-end group or a R′C(═O)—N-linking group, as these phrases are defined hereinabove, where R′ and R″ are as defined herein.

The term “hydrazine” describes a —NR′—NR″R″′ end group or a —NR′—NR″— linking group, as these phrases are defined hereinabove, with R′, R″, and R″′ as defined herein.

As used herein, the term “hydrazide” describes a —C(═O)—NR′—NR″R″′ end group or a —C(═O)—NR′—NR″— linking group, as these phrases are defined hereinabove, where R′, R″ and R′″ are as defined herein.

As used herein, the term “thiohydrazide” describes a —C(═S)—NR′—NR″R″′ end group or a —C(═S)—NR′—NR″— linking group, as these phrases are defined hereinabove, where R′, R″ and R″′ are as defined herein.

Processes:

Further according to embodiments of the present invention, there are provided processes of preparing the compounds as described herein.

These processes are generally effected by providing a paromamine derivative and introducing thereto a desired modification to thereby obtain a pseudo-disaccharide compound as described herein.

Processes of preparing pseudo-trisacchride compounds as described herein are generally effected by devising appropriate acceptor aminoglycoside molecules and corresponding donor molecules, as is known in the art of aminoglycosides.

Generally, the synthesis of pseudo-trisaccharide compounds according to some embodiments of the present invention is accomplished using suitable acceptor and donor molecules and reaction conditions which allow reacting a protected derivative of the donor and of the acceptor and removing the protecting group so as to obtain a desired pseudo-trisaccharide of Formula Ia or IIIa.

The term “acceptor” is used herein to describe the skeletal structure derived from paromamine which has an available (unprotected) hydroxyl group at position C3′, C4′, C6 or C5, preferably C5, which is reactive during a glycosylation reaction, and can accept a glycosyl.

The term “donor” is used herein to describe the glycosyl that reacts with the acceptor to form the final pseudo-trisaccharide compound.

The term “glycosyl”, as used herein, refers to a chemical group which is obtained by removing the hydroxyl group from the hemiacetal function of a monosaccharide.

The donors and acceptors are designed so as to form the desired compounds according to some embodiments of the present invention. The following describes some embodiments of this aspect of the present invention, presenting exemplary processes of preparing exemplary subsets of the compounds described herein. More detailed processes of preparing exemplary compounds according to some embodiments of the present invention, are presented in the Examples section that follows below.

The syntheses of pseudo-trisaccharide compounds according to some embodiments of the present invention, generally include (i) preparing an acceptor compound by selective protection of one or more hydroxyls and amines at selected positions present on the paromamine scaffold, leaving the selected position (e.g., C5) unprotected and therefore free to accept a donor (glycosyl) compound as defined herein; (ii) preparing a donor compound by selective protection of one or more hydroxyls and amines at selected positions present on the glycosyl, leaving one position unprotected and therefore free to couple with an acceptor compound as defined herein; (iii) subjecting the donor and the acceptor to a coupling reaction; and (iii) removing the protecting groups to thereby obtain the desired compound.

The phrase “protected group”, as used herein, refers to a group that is substituted or modified so as to block its functionality and protect it from reacting with other groups under the reaction conditions (e.g., a coupling reaction as described herein). A protected group is re-generated by removal of the substituent or by being re-modified.

When an “amino-protected group” or “hydroxyl-protected group” are used, it is meant that a protecting group is attached or used to modify the respective group so as to generate the protected group.

The phrase “protecting group”, as used herein, refers to a substituent or a modification that is commonly employed to block or protect a particular functionality while reacting other functional groups on the compound. The protecting group is selected so as to release the substituent or to be re-modified, to thereby generate the desired unprotected group.

For example, an “amino-protecting group” or “amine-protecting group” is a substituent attached to an amino group, or a modification of an amino group, that blocks or protects the amino functionality in the compound, and prevents it from participating in chemical reactions. The amino-protecting group is removed by removal of the substituent or by a modification that re-generates an amine group.

Suitable amino-protected groups include azide (azido), N-phthalimido, N-acetyl, N-trifluoroacetyl, N-t-butoxycarbonyl (BOC), N-benzyloxycarbonyl (CBz) and N-9-fluorenylmethylenoxycarbonyl (Fmoc).

A “hydroxyl-protecting group” or “hydroxyl-protecting group” refers to a substituent or a modification of a hydroxyl group that blocks or protects the hydroxyl functionality, and prevents it from participating in chemical reactions. The hydroxy-protecting group is removed by removal of the substituent or by a modification that re-generates a hydroxy group.

Suitable hydroxy protected groups include isopropylidene ketal and cyclohexanone dimethyl ketal (forming a 1,3-dioxane with two adjacent hydroxyl groups), 4-methoxy-1-methylbenzene (forming a 1,3-dioxane with two adjacent hydroxyl groups), O-acetyl, O-chloroacetyl, O-benzoyl and O-silyl.

For a general description of protecting groups and their use, see T. W. Greene, Protective Groups in Organic Synthesis, John Wiley & Sons, New York, 1991.

According to some embodiments, the amino-protected groups include an azido (N₃-) and/or an N-phthalimido group, and the hydroxyl-protecting groups include O-acetyl (AcO—), O-benzoyl (BzO—) and/or O-chloroacetyl.

It is noted herein that when applicable, a “protected group” refers to a moiety in which one reactive function on a compound is protected or more than one function are protected at the same time, such as in the case of two adjacent functionalities, e.g., two hydroxyl groups that can be protected at once by a isopropylidene ketal.

In some embodiments, the donor compound is a protected monosaccharide which can be represented by the general Formula IV: In some embodiments, the donor compound is a protected monosaccharide which can be represented by the general Formula IV, having a leaving group at position 1″ thereof, denoted L, and optionally a substituent R₁₂ at position 5″, as defined herein:

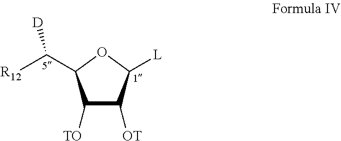

wherein:

L is a leaving group;

OT is a donor protected hydroxyl group;

R₁₂ is as defined herein for Formula II, Ia or IIIa (the configuration at the 5″ position as presented in Formula IV being a non-limiting example); and

D is a protected or unprotected form of NR₁₄R₁₅ as defined for Formula II, Ia or IIIa, wherein when R₁₄ and R₁₅ are both hydrogen, D is a donor protected amine group.

As used herein, the phrase “leaving group” describes a labile atom, group or chemical moiety that readily undergoes detachment from an organic molecule during a chemical reaction, while the detachment is typically facilitated by the relative stability of the leaving atom, group or moiety thereupon. Typically, any group that is the conjugate base of a strong acid can act as a leaving group. Representative examples of suitable leaving groups according to some of the present embodiments include, without limitation, trichloroacetimidate, acetate, tosylate, triflate, sulfonate, azide, halide, hydroxy, thiohydroxy, alkoxy, cyanate, thiocyanate, nitro and cyano.

According to some embodiments of the present invention, each of the donor hydroxyl-protecting groups is O-benzoyl and the donor amino-protecting group in either R₁₅ or R₁₄ is azido, although other protecting groups are contemplated.

It is to be noted that when one of R₁₄ and R₁₅ is other than hydrogen, it can be protected or unprotected. Typically, when one of R₁₄ and R₁₅ is guanine or guanidine, a protecting group suitable for the reaction conditions (e.g., of a coupling reaction with an acceptor) can be used. Optionally, the guanine or guanidine are unprotected. When one of R₁₄ and R₁₅ is an alkyl, aryl or cycloalkyl, typically D in Formula IV is an unprotected form of NR₁₄R₁₅.

The structure of the donor compound sets the absolute structure of Ring III in the resulting compound according to some embodiments of the present invention, namely the stereo-configuration of the 5″ position and the type of R₁₄, R₁₅ and R₁₂ in Formula II, Ia or IIIa.

Exemplary acceptor molecules suitable for use in the preparation of the compounds described herein under Formula I or Ia, are represented by Formula V:

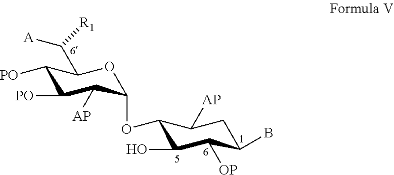

wherein:

the dashed line represents an S-configuration or an R-configuration at position 6′;

OP is an acceptor protected hydroxyl group;

AP is an acceptor protected amine group;

R₁ is as defined herein for Formula I or Ia;

A is an acceptor protected hydroxyl group (OP); or can otherwise be one of the other groups defining OR₂, either protected or unprotected, according to the chemical nature of these groups and the reaction conditions; and

B is an acceptor protected amine group, in case R₇ is Formula Ia is hydrogen, or can otherwise be a protected or unprotected form of the groups defining R₇.

According to some embodiments of the present invention, the acceptor hydroxyl-protected group is O-acetyl.

According to some embodiments of the present invention, the donor amino-protecting group is azido, although other protecting groups are contemplated.

The acceptor hydroxyl-protected groups and the acceptor amino-protected groups at the various positions of the acceptors can be the same or different each position.

In some embodiments, for example, in case R₇ is other than H, the acceptor is prepared by generating the moiety B, prior to reacting it with the donor.

The structure of the acceptor compound sets the absolute structure of Ring I and Ring II in the resulting compound according to some embodiments of the present invention.

In some embodiments, the synthesis of pseudo-disaccharide compounds of Formula I, according to some embodiments of the present invention, is accomplished using an amino-protected compound of Formula VI:

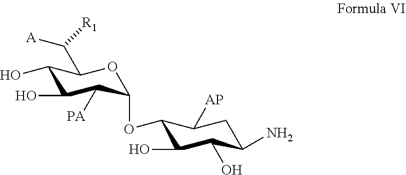

wherein:

the dashed line represents an S-configuration or an R-configuration at position 6′;

AP is an acceptor protected amine group;

R₁ is as defined herein for Formula Ia;

A is an acceptor protected hydroxyl group (OP), as described herein; or can otherwise be one of the other groups defining OR₂, either protected or unprotected, according to the chemical nature of these groups and the reaction conditions.

Exemplary acceptors suitable for use in preparing compounds of Formulae III or IIIa are presented in the Examples section that follows.

Embodiments of the present invention further encompass any of the intermediate compounds described herein in the context of processes of preparing the compounds of the present embodiments.

Therapeutic Uses:

As known in the art, about a third of alleles causing genetic diseases carry premature termination (stop) codons (PTCs), which lead to the production of truncated proteins. One possible therapeutic approach involves the induction and/or promotion of readthrough of such PTCs to enable synthesis of full-length proteins. PTCs originate from either mutations, such as nonsense mutations, frame-shift deletions and insertions, or from aberrant splicing that generates mRNA isoforms with truncated reading frames. These mutations can lead to the production of truncated, nonfunctional or deleterious proteins, owing to dominantnegative or gain-of-function effects.

In general, readthrough of PTCs can be achieved by suppressor transfer RNAs (tRNAs), factors that decrease translation-termination efficiency, such as small-interfering RNAs (siRNAs) directed against the translation-termination factors, and RNA antisense that targets the nonsense mutation region. One of the objectives of the present invention is to provide a pharmacological therapeutic approach aimed at achieving sufficient levels of functional proteins in a subject suffering from at least one genetic disorder associated with at least one premature stop-codon mutation. According to embodiments of the present invention, the provided therapeutic approach is aimed at inducing and/or promoting translational readthrough of the disease causing PTCs, to enable the synthesis and expression of full-length functional proteins.

As presented hereinabove, one extensively studied approach that has reached clinical trials, is based on readthrough by drugs affecting the ribosome decoding site, such as aminoglycoside antibiotics; however, aminoglycosides have severe adverse side effects when used at high concentrations and/or used long-term. The compounds presented herein were designed to exhibit an ability to induce and/or promote readthrough of a premature stop-codon mutation in an organism having such a mutation, while exhibiting minimal adverse effects. Such an activity renders these compounds suitable for use as therapeutically active agents for the treatment of genetic disorders associated with a premature stop-codon mutation.

As demonstrated in the Examples section that follows, exemplary compounds encompassed by the present embodiments were indeed shown to exhibit a premature stop-codon mutation suppression activity, and hence as useful in inducing readthrough of genes characterized by a disease-causing premature stop-codon mutation, and thus exhibit usefulness in treating respective genetic diseases or disorders associated with a premature stop-codon mutation.

According to an aspect of some embodiments of the present invention, any of the compounds presented herein having Formula I, la, III or IIIa, including any of the respective embodiments of the compounds and any combinations thereof, are for use in inducing and/or promoting readthrough of a premature stop codon mutation and/or for increasing an expression of a gene having a premature stop codon mutation, and/or are for use in the manufacture of a medicament for inducing and/or promoting readthrough of a premature stop codon mutation and/or for increasing an expression of a gene having a premature stop codon mutation.

According to an aspect of some embodiments of the present invention, any of the compounds presented herein having Formula I, Ia, III or IIIa, including any of the respective embodiments of the compounds and any combinations thereof, are for use in the treatment of a genetic disorder associated with a premature stop-codon mutation, or for use in the manufacture of a medicament for the treatment of a genetic disorder associated with a premature stop-codon mutation.

Any of the premature stop-codon mutations are contemplated. In some embodiments, the mutations are those having an RNA code of UGA, UAG or UAA.

According to some of any of the embodiments described herein, the protein is translated in a cytoplasmic translation system.

According to some of any of the embodiments described herein, the compound is used in a mutation suppression amount.

According to some of any of the embodiments described herein, an inhibition of translation IC₅₀ of the compound in a eukaryotic cytoplasmic translation system is greater that an inhibition of translation IC₅₀ of the compound in a ribosomal translation system.

According to some of any of the embodiments described herein, an inhibition of translation IC₅₀ of the compound in a eukaryotic cytoplasmic translation system is greater that an inhibition of translation IC₅₀ of the compound in a prokaryotic translation system.

According to an aspect of some embodiments of the present invention, any of the compounds presented herein having Formula I, Ia, III or IIIa, including any of the respective embodiments of the compounds and any combinations thereof, are for use in the treatment of a genetic disorder associated with a premature stop-codon mutation, or for use in the manufacture of a medicament for the treatment of a genetic disorder associated with a premature stop-codon mutation.

According to an aspect of some embodiments of the present invention there is provided a method of treating a genetic disorder associated with a premature stop-codon mutation. The method, according to this aspect of the present invention, is effected by administering to a subject in need thereof a therapeutically effective amount of one or more of the compounds presented herein having Formula I, Ia, III or IIIa, including any of the respective embodiments of the compounds and any combinations thereof.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

As used herein, the phrase “therapeutically effective amount” describes an amount of the polymer being administered which will relieve to some extent one or more of the symptoms of the condition being treated.

The phrase “genetic disorder”, as used herein, refers to a chronic disorder which is caused by one or more defective genes that are often inherited from the parents, and which can occur unexpectedly when two healthy carriers of a defective recessive gene reproduce, or when the defective gene is dominant. Genetic disorders can occur in different inheritance patterns which include the autosomal dominant pattern wherein only one mutated copy of the gene is needed for an offspring to be affected, and the autosomal recessive pattern wherein two copies of the gene must be mutated for an offspring to be affected.

The phrase “genetic disorder”, as used herein, encompasses a genetic disorder, genetic disease, genetic condition or genetic syndrome.

According to some of any of the embodiments of the present invention, the genetic disorder, genetic disease, genetic condition or genetic syndrome, involves a gene having a premature stop-codon mutation, also referred to herein as a truncation mutation and/or a nonsense mutation, which leads to improper translation thereof. The improper translation produces a dysfunctional essential protein or causes a reduction or abolishment of synthesis of an essential protein. In the context of the some embodiments of the present invention, the genetic disorders which are contemplated within the scope of the present embodiments are referred to as genetic disorders associated with a premature stop-codon mutation and/or a protein truncation phenotype.

According to some of any of the embodiments of the present invention, a genetic disorder associated with a premature stop-codon mutation and/or a protein truncation phenotype is treatable by inducing and/or promoting readthrough of the mutation in the complete but otherwise defective transcript (mRNA), or in other words, by inducing and/or promoting suppression of the nonsense mutation (the premature stop-codon mutation and/or the truncation mutation). In the context of embodiments of the present invention, a genetic disorder is one that is treatable by readthrough-inducing and/or promoting compounds.

Methods for identification of a genetic disorder associated with a premature stop-codon mutation and/or a protein truncation phenotype are well known in the art, and include full or partial genome elucidation, genetic biomarker detection, phenotype classification and hereditary information analysis.

Such methods often result in pairs of mutant/wild type (WT) sequences, and these pairs can be used in known methodologies for identifying if the genetic disorder is associated with a premature stop-codon mutation and/or a protein truncation phenotype.

A readthrough-inducing/promoting activity of compounds for treating such genetic disorders can be established by methods well known in the art.

For example, a plasmid comprising two reporter genes interrupted by a sequence of the mutated gene (the genetic disorder-causing gene) is transected into a protein expression platform, either in full cells or in a cell-free systems, and the ratio between the expression level of the two genes in the presence of a tested compound is measured, typically in series of concentrations and duplications, and compared to the gene expression level ratio of the wild-type and/or to the expression level ratio measured in a control sample not containing the tested compound.

It is noted that the experimental model for readthrough activity, namely the nucleotide sequence of gene containing the premature stop-codon mutation, is a byproduct of the process of identifying a genetic disorder as associated with a premature stop-codon mutation and/or a protein truncation phenotype, and further noted that with the great advances in genomic data acquisition, this process is now well within the skills of the artisans of the art, and that once the mechanism of action of a drug candidate is established, as in the case of genetic disorders which have been shown to be associated with a premature stop-codon mutation and/or a protein truncation phenotype, it is well within the skills of the artisans of the art to identify, characterize and assess the efficacy, selectivity and safety of any one of the readthrough-inducing compounds presented herein. It is further well within the skills of the artisans of the art to take the readthrough-inducing compounds presented herein further though the routine processes of drug development.

Methodologies for testing readthrough of a premature stop-codon mutation and/or a truncation mutation, referred to herein as readthrough activity, are known in the art, and several exemplary experimental methods are provided in the Examples section that follows, by which the readthrough-inducing compounds, according to some embodiments of the present invention, can be characterized. It is to be understood that other methods can be used to characterized readthrough-inducing compounds, and such methods are also contemplated within the scope of the present invention. Methods such as provided herein can also be adapted for high throughput screening technology that can assay thousands of compounds in a relatively short period of time.

The skilled artisan would appreciate that many in vitro methodologies can be used to characterize readthrough-inducing compounds provided herein in terms of safety of use as drugs, and assess the drug candidates in terms of their cytotoxicity versus their efficacy. The skilled artisan would also appreciate that many in vitro methodologies can be used to characterize the readthrough-inducing compounds provided herein for eukaryotic versus prokaryotic selectivity, and such methodologies may also be adapted for high throughput screening technology that can assay thousands of compounds in a relatively short period of time.

Non-limiting examples of genetic disorders, diseases, conditions and syndromes, which are associated with the presence of at least one premature stop-codon or other nonsense mutations include cancer, Rett syndrome, cystic fibrosis (CF), Becker's muscular dystrophy (BMD), Congenital muscular dystrophy (CMD), Duchenne muscular dystrophy (DMD), Factor VII deficiency, Familial atrial fibrillation, Hailey-Hailey disease, hemophilia A, hemophilia B, Hurler syndrome, Louis-Bar syndrome (ataxia-telangiectasia, AT), McArdle disease, Mucopolysaccharidosis, Nephropathic cystinosis, Polycystic kidney disease, type I, II and III Spinal muscular atrophy (SMA), Tay-Sachs, Usher syndrome, cystinosis, Severe epidermolysis bullosa, Dravet syndrome, X-linked nephrogenic diabetes insipidus (XNDI) and X-linked retinitis pigmentosa.

Additional genetic disorders, diseases, conditions and syndromes, which are associated with the presence of at least one premature stop-codon or other nonsense mutations, are listed in “Suppression of nonsense mutations as a therapeutic approach to treat genetic diseases” by Kim M. Keeling, K. M Bedwell, D.M., Wiley Interdisciplinary Reviews: RNA, 2011, 2(6), p. 837-852; “Cancer syndromes and therapy by stop-codon readthrough”, by Bordeira-Carriço, R. et al., Trends in Molecular Medicine, 2012, 18(11), p. 667-678, and any references cited therein, all of which are incorporated herewith by reference in their entirety.

In any of the methods and uses described herein, the compounds described herein can be utilized either per se or form a part of a pharmaceutical composition, which further comprises a pharmaceutically acceptable carrier, as defined herein.

According to an aspect of some embodiments of the present invention, there is provided a pharmaceutical composition which comprises, as an active ingredient, any of the novel compounds described herein and a pharmaceutically acceptable carrier.

As used herein a “pharmaceutical composition” refers to a preparation of the compounds presented herein, with other chemical components such as pharmaceutically acceptable and suitable carriers and excipients. The purpose of a pharmaceutical composition is to facilitate administration of a compound to an organism.

Hereinafter, the term “pharmaceutically acceptable carrier” refers to a carrier or a diluent that does not cause significant irritation to an organism and does not abrogate the biological activity and properties of the administered compound. Examples, without limitations, of carriers are: propylene glycol, saline, emulsions and mixtures of organic solvents with water, as well as solid (e.g., powdered) and gaseous carriers.

Herein the term “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences” Mack Publishing Co., Easton, Pa., latest edition, which is incorporated herein by reference.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more pharmaceutically acceptable carriers comprising excipients and auxiliaries, which facilitate processing of the compounds presented herein into preparations which, can be used pharmaceutically. Proper formulation is dependent upon the route of administration chosen.

According to some embodiments, the administration is effected orally. For oral administration, the compounds presented herein can be formulated readily by combining the compounds with pharmaceutically acceptable carriers well known in the art. Such carriers enable the compounds presented herein to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the compounds presented herein may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For injection, the compounds presented herein may be formulated in aqueous solutions, preferably in physiologically compatible buffers such as Hank's solution, Ringer's solution, or physiological saline buffer with or without organic solvents such as propylene glycol, polyethylene glycol.

For transmucosal administration, penetrants are used in the formulation. Such penetrants are generally known in the art.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active aminoglycoside compounds doses.

For buccal administration, the compositions may take the form of tablets or lozenges formulated in conventional manner.

For administration by inhalation, the compounds presented herein are conveniently delivered in the form of an aerosol spray presentation (which typically includes powdered, liquefied and/or gaseous carriers) from a pressurized pack or a nebulizer, with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the compounds presented herein and a suitable powder base such as, but not limited to, lactose or starch.

The compounds presented herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the compounds preparation in water-soluble form. Additionally, suspensions of the compounds presented herein may be prepared as appropriate oily injection suspensions and emulsions (e.g., water-in-oil, oil-in-water or water-in-oil in oil emulsions). Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents, which increase the solubility of the compounds presented herein to allow for the preparation of highly concentrated solutions.

Alternatively, the compounds presented herein may be in powder form for constitution with a suitable vehicle, e.g., sterile, pyrogen-free water, before use.

The compounds presented herein may also be formulated in rectal compositions such as suppositories or retention enemas, using, e.g., conventional suppository bases such as cocoa butter or other glycerides.

The pharmaceutical compositions herein described may also comprise suitable solid of gel phase carriers or excipients. Examples of such carriers or excipients include, but are not limited to, calcium carbonate, calcium phosphate, various sugars, starches, cellulose derivatives, gelatin and polymers such as polyethylene glycols.

Pharmaceutical compositions suitable for use in context of the present invention include compositions wherein the active ingredients are contained in an amount effective to achieve the intended purpose. More specifically, a therapeutically effective amount means an amount of compounds presented herein effective to prevent, alleviate or ameliorate symptoms of the disorder, or prolong the survival of the subject being treated.

Determination of a therapeutically effective amount is well within the capability of those skilled in the art, especially in light of the detailed disclosure provided herein.

For any compounds presented herein used in the methods of the present embodiments, the therapeutically effective amount or dose can be estimated initially from activity assays in animals. For example, a dose can be formulated in animal models to achieve a circulating concentration range that includes the mutation suppression levels as determined by activity assays (e.g., the concentration of the test compounds which achieves a substantial read-through of the truncation mutation). Such information can be used to more accurately determine useful doses in humans.

Toxicity and therapeutic efficacy of the compounds presented herein can be determined by standard pharmaceutical procedures in experimental animals, e.g., by determining the EC₅₀ (the concentration of a compound where 50% of its maximal effect is observed) and the LD₅₀ (lethal dose causing death in 50% of the tested animals) for a subject compound. The data obtained from these activity assays and animal studies can be used in formulating a range of dosage for use in human.

The dosage may vary depending upon the dosage form employed and the route of administration utilized. The exact formulation, route of administration and dosage can be chosen by the individual physician in view of the patient's condition. (See e.g., Fingl et al., 1975, in “The Pharmacological Basis of Therapeutics”, Ch. 1 p. 1).

Dosage amount and interval may be adjusted individually to provide plasma levels of the compounds presented herein which are sufficient to maintain the desired effects, termed the minimal effective concentration (MEC). The MEC will vary for each preparation, but can be estimated from in vitro data; e.g., the concentration of the compounds necessary to achieve 50-90% expression of the whole gene having a truncation mutation, i.e. read-through of the mutation codon. Dosages necessary to achieve the MEC will depend on individual characteristics and route of administration. HPLC assays or bioassays can be used to determine plasma concentrations.

Dosage intervals can also be determined using the MEC value. Preparations should be administered using a regimen, which maintains plasma levels above the MEC for 10-90% of the time, preferable between 30-90% and most preferably 50-90%.

Depending on the severity and responsiveness of the chronic condition to be treated, dosing can also be a single periodic administration of a slow release composition described hereinabove, with course of periodic treatment lasting from several days to several weeks or until sufficient amelioration is effected during the periodic treatment or substantial diminution of the disorder state is achieved for the periodic treatment.

The amount of a composition to be administered will, of course, be dependent on the subject being treated, the severity of the affliction, the manner of administration, the judgment of the prescribing physician, etc. Compositions of the present invention may, if desired, be presented in a pack or dispenser device, such as an FDA (the U.S. Food and Drug Administration) approved kit, which may contain one or more unit dosage forms containing the active ingredient. The pack may, for example, comprise metal or plastic foil, such as, but not limited to a blister pack or a pressurized container (for inhalation). The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions for human or veterinary administration. Such notice, for example, may be of labeling approved by the U.S. Food and Drug Administration for prescription drugs or of an approved product insert. Compositions comprising a compound according to the present embodiments, formulated in a compatible pharmaceutical carrier may also be prepared, placed in an appropriate container, and labeled for treatment of an indicated condition or diagnosis, as is detailed hereinabove.

Thus, in some embodiments, the pharmaceutical composition is packaged in a packaging material and identified in print, in or on the packaging material, for use in the treatment of a genetic disorder, as defined herein, and/or in any of the uses described herein.

In some embodiments, the pharmaceutical composition is for use in the treatment of a genetic disorder, as defined herein, and/or in any of the uses described herein.

In any of the composition, methods and uses described herein, the compounds can be utilized in combination with other agents useful in the treatment of the genetic disorder and/or in inducing or promoting readthrough activity of a premature stop codon mutation and/or in increasing expression of a gene having a premature stop codon mutation as described herein.

Being primarily directed at treating genetic disorders, which are chronic by definition, the compounds presented herein or pharmaceutical compositions containing the same are expected to be administered throughout the lifetime of the subject being treated. Therefore, the mode of administration of pharmaceutical compositions containing the compounds should be such that will be easy and comfortable for administration, preferably by self-administration, and such that will take the smallest toll on the patient's wellbeing and course of life.

The repetitive and periodic administration of the compounds presented herein or the pharmaceutical compositions containing the same can be effected, for example, on a daily basis, i.e. once a day, more preferably the administration is effected on a weekly basis, i.e. once a week, more preferably the administration is effected on a monthly basis, i.e. once a month, and most preferably the administration is effected once every several months (e.g., every 1.5 months, 2 months, 3 months, 4 months, 5 months, or even 6 months).

As discussed hereinabove, some of the limitations for using presently known aminoglycosides as truncation mutation readthrough drugs are associated with the fact that they are primarily antibacterial (used as antibiotic agents). Chronic use of any antibacterial agents is highly unwarranted and even life threatening as it alters intestinal microbial flora which may cause or worsen other medical conditions such as flaring of inflammatory bowel disease, and may cause the emergence of resistance in some pathological strains of microorganisms.

In some embodiments, the compounds presented herein have substantially no antibacterial activity. By “no antibacterial activity” it is meant that the minimal inhibition concentration (MIC) thereof for a particular strain is much higher than the concentration of a compound that is considered an antibiotic with respect to this strain. Further, the MIC of these compounds is notably higher than the concentration required for exerting truncation mutation suppression activity.

Being substantially non-bactericidal, the compounds presented herein do not exert the aforementioned adverse effects and hence can be administered via absorption paths that may contain benign and/or beneficial microorganisms that are not targeted and thus their preservation may even be required. This important characteristic of the compounds presented herein renders these compounds particularly effective drugs against chronic conditions since they can be administered repetitively and during life time, without causing any antibacterial-related adverse, accumulating effects, and can further be administered orally or rectally, i.e. via the GI tract, which is a very helpful and important characteristic for a drug directed at treating chronic disorders.

According to some embodiments, the compounds presented herein are selected and/or designed to be selective towards the eukaryotic cellular translation system versus that of prokaryotic cells, namely the compounds exhibit higher activity in eukaryotic cells, such as those of mammalian (humans) as compared to their activity in prokaryotic cells, such as those of bacteria. Without being bound by any particular theory, it is assumed that the compounds presented herein, which are known to act by binding to the A-site of the 16S ribosomal RNA while the ribosome is involved in translating a gene, have a higher affinity to the eukaryotic ribosomal A-site, or otherwise are selective towards the eukaryotic A-site, versus the prokaryotic ribosomal A-site, as well as the mitochondrial ribosomal A-site which resembles its prokaryotic counterpart.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof. Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is expected that during the life of a patent maturing from this application many relevant genetic diseases and disorders as defined herein will be uncovered and the scope of this term is intended to include all such new disorders and diseases a priori.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Example 1

### Chemical Syntheses of Exemplary Diol-Containing Aminoglycosides According to Some Embodiments of the Present Invention

General Techniques:

NMR spectra (including 1H, 13C, DEPT, 2D-COSY, 1D TOCSY, HMQC, HMBC) were routinely recorded on a Bruker Avance™ 500 spectrometer, and chemical shifts reported (in ppm) are relative to internal Me₄Si (δ=0.0) with CDCl₃ as the solvent, and to MeOD (δ=3.35) as the solvent. ¹³C NMR spectra were recorded on a Bruker Avance™ 500 spectrometer at 125.8 MHz, and the chemical shifts reported (in ppm) relative to the solvent signal for CDCl₃ (δ=77.00), or to the solvent signal for MeOD (δ=49.0).

Mass spectra analyses were obtained either on a Bruker Daltonix Apex 3 mass spectrometer under electron spray ionization (ESI) or by a TSQ-70B mass spectrometer (Finnigan Mat).

Reactions were monitored by TLC on Silica Gel 60 F254 (0.25 mm, Merck), and spots were visualized by charring with a yellow solution containing (NH₄)Mo₇O₂₄.4H₂O (120 grams) and (NH₄)₂Ce(NO₃)₆ (5 grams) in 10% H₂SO₄ (800 mL).

Flash column chromatography was performed on Silica Gel 60 (70-230 mesh).

All reactions were carried out under an argon atmosphere with anhydrous solvents, unless otherwise indicated.

G418 (geneticin) and gentamicin were purchased from Sigma. All other chemicals and biochemicals, unless otherwise indicated, were obtained from commercial sources.

Compounds NB153, NB 155, NB156 and NB157, presented in Table 1 below, are prepared essentially as described hereinabove and in further detail hereinbelow.

All the structures were confirmed by a combination of various 1D and 2D NMR techniques, including 1D TOCSY, 2D COSY, 2D ¹H-¹³C HMQC and HMBC, along with mass spectrometry.

TABLE 1

| Compound | Structure |
|---|---|
| NB153 | 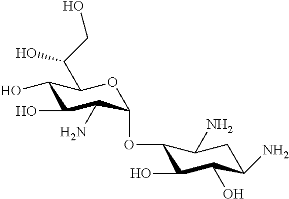 |
| NB155 | 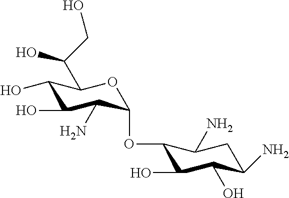 |
| NB156 | 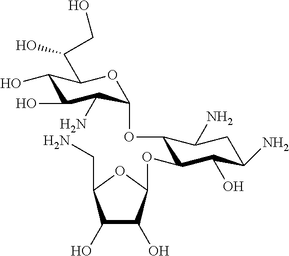 |
| NB157 | 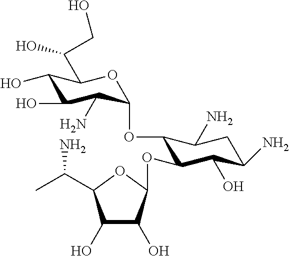 |

Syntheses of Pseudo-Disaccharides NB153 and NB155:

NB153 and NB155 pseudo-disaccharides are two diastereomers at C6′ position of the 6′,7′-diol, exhibiting 6′-(R) configuration and 6′-(S) configuration, respectively.

The syntheses of compounds NB153 and NB155 are illustrated in Scheme 1 below.

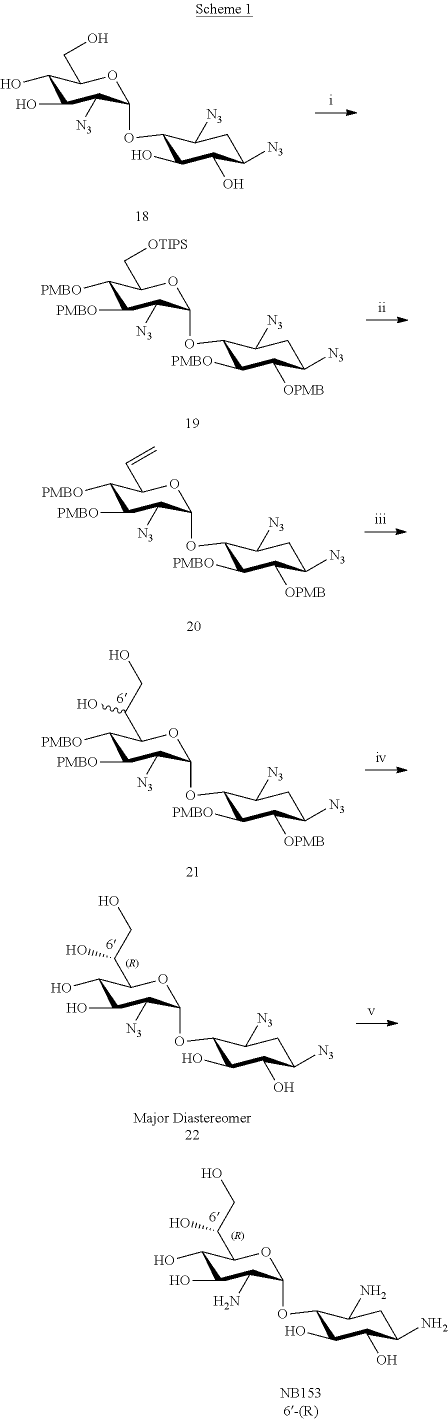

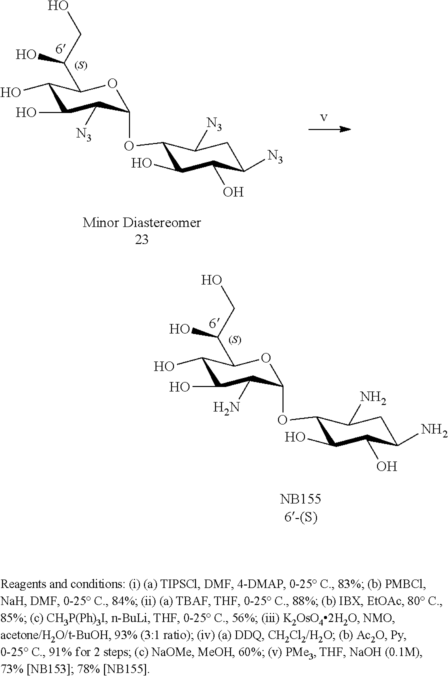

Briefly, the perazido derivative 18 was selectively protected by TIPSCl, and the remaining hydroxyls were protected by pmethoxybenzyl (PMB) groups to afford 19. Selective deprotection of silyl group with TBAF was followed by oxidation with 2-iodoxy benzoic acid (IBX) and Wittig reaction to afford the terminal alkene 20. The alkene 20 was dihydroxylated to provide the diol 21 as an inseparable mixture of 6′-diastereomers. Treatment of 21 with DDQ was followed by acetylation (Ac₂O) and deacetylation (NaOMe) steps to afford the mixture of 6′-diastereomers (about 3:1 ratio), which was successfully separated by column chromatography to give the major diastereomer 22 and the minor diastereomer 23. The absolute configuration at 6′-position was assigned by using ¹H-NMR magnetic anisotropy, as detailed hereinbelow, which established 6-(R)-configuration and 6-(S)-configuration for the major and minor diastereomers, respectively. The two diastereomers 22 and 23 were separately subjected to Staudinger reaction to produce the pseudodisaccharides NB153 and NB155, respectively.

### Synthesis of (2R,3S,4R,5R,6S)-5-azido-6-(((1R,2R,3S,4R,6S)-4,6-diazido-2,3-dihydroxycyclohexyl)oxy)-2-(hydroxymethyl)tetrahydro-2H-pyran-3,4-diol (Compound 18)

Compound 18 was prepared according to previously published procedure [Nyffeler et al. J. Am. Chem. Soc. 2002, 124, 10773-10778]. Briefly, the paromamine (1.0 gram, 3.0 mmol), NaHCO₃ (3.1 grams, 36.9 mmol) and copper (II) sulfate (6 mg, 0.24 mmol) were dissolved in water (5.0 mL). Triflic azide stock solution prepared from Tf₂O (4.6 mL, 27.6 mmol) and NaN₃ (3.6 grams, 55.7 mmol) was added followed by the addition of methanol (40 mL) to reach the homogeneous solution. The reaction mixture (blue color) was stirred vigorously at room temperature and the completion of the reaction was monitored by the change of blue color to green. After stirring for 48 hours, TLC (EtOAc/MeOH 95:5) analysis indicated the completion of the reaction. The solvents were evaporated to dryness and the residue was subjected to column chromatography (EtOAc 100%) to thereby obtain compound 18 (650 mg, 52% yield).

¹H NMR (500 MHz, MeOD): ‘Ring I’: δ=H 5.69 (d, 1H, J=3.7 Hz, H-1), 3.99 (ddd, 1H, J=9.9, 4.1, 2.6 Hz, H-5), 3.94 (dd, 1H, J=10.2, 9.1 Hz, H-3), 3.84 (dd, 1H, J=11.9, 2.3 Hz, H-6), 3.78 (dd, 1H, J=11.8, 4.4 Hz, H-6), 3.46 (dd, 1H, J=9.7, 9.3 Hz, H-4), 3.13 (dd, 1H, J=10.5, 3.7 Hz, H-2); ‘Ring II’: δH 3.80 (t, 1H, J=8.8 Hz, H-5), 3.77-3.67 (m, 3H, H-1, H-3, H-4), 3.56 (t, 1H, J=9.6 Hz, H-6), 2.59-2.48 (m, 1H), 1.68 (dd, 1H, J=26.3, 12.7 Hz, H-2).

¹³C NMR (125 MHz, MeOD): δ=C 99.3 (C1′), 80.7, 77.8 (C5), 77.7 (C6), 73.9 (C5′), 72.4 (C3′), 71.6, 64.8 (C2′), 62.1 (C6′), 61.6, 60.9, 33.1 (C2).

MALDI TOFMS: calculated for C₁₂H₁₉N₉O₇ ([M+K]+) m/e 440.3; measured m/e 440.2.

### Synthesis of (((2R,3S,4R,5R,6S)-5-azido-6-(((1R,2R,3S,4R,6S)-4,6-diazido-2,3-bis((4-methoxybenzyl)oxy)cyclohexyl)oxy)-3,4-bis((4-methoxybenzyl) oxy)tetrahydro-2H-pyran-2-yl)methoxy)triisopropylsilane (Compound 19)

Compound 18 (11.6 grams, 28.9 mmol) was dissolved in anhydrous DMF (80 mL) and cooled to 0° C. Triisopropylsilyl chloride (TIPSCl, 8 mL, 37.3 mmol) was added dropwise, followed by addition of 4-DMAP (10.6 grams, 86.7 mmol). The reaction mixture was allowed to attain the room temperature under stirring, and the reaction progress was monitored by TLC (EtOAc/Hexane 7:3), which indicated the completion after 5 hours. The reaction mixture was diluted with ethyl acetate (50 mL) and H₂O (20 mL), and the two layers were separated. The aqueous layer was thoroughly washed with ethyl acetate (4×30 mL). The combined organic layers were washed with sat. NaCl solution and dried over anhydrous MgSO₄. The solvent was evaporated to dryness and the residue was subjected to column chromatography (EtOAc/Hexane 25:75) to yield the corresponding silyl ether (18a) (13.3 grams, 83%).

¹H NMR (500 MHz, CDCl₃): ‘Ring I’: δ=H 5.14 (d, 1H, J=4.0 Hz, H-1), 4.09-4.02 (m, 2H, H-3, H-6), 3.98 (td, 1H, J1=8.0, J2=4.5 Hz, H-5), 3.82 (dd, 1H, J1=9.5, J2=8.0 Hz, H-6), 3.66 (t, 1H, J=9.0 Hz, H-4), 3.48 (dd, 1H, J1=10.5, J2=4.0 Hz, H-2); ‘Ring II’: δ=H 3.52 (t, 1H, J=8.0 Hz, H-5), 3.47-3.37 (m, 2H, H-1, H-6), 3.34-3.22 (m, 2H, H-3, H-4), 2.29 (dt, 1H, J1=12.0, J2=4.0 Hz, H-2eq), 1.47 (ddd, 1H, J1=J2=J3=12.0 Hz, H-2ax); The additional peaks in the spectrum were identified as follow: δ=H 1.16-1.09 (m, 3H, TIPS), 1.07 (s, 12H, TIPS), 1.06 (s, 6H, TIPS).

¹³C NMR (125 MHz, CDCl₃): δ=C 99.3 (C1′), 83.4 (C4), 76.1 (C5), 75.5 (C6), 75.1 (C4′), 72.6 (C3′), 69.6 (C5′), 66.0 (C6′), 63.5 (C2′), 59.8 (C1), 58.9 (C3), 32.1 (C2), 17.9 (2C, TIPS), 11.8 (TIPS).

MALDI TOFMS: calculated for C₂₁H₃₉N₉O₇Si ([M+Na]+) m/e 580.6; measured m/e 580.3.

To a stirred solution of the silyl ether from above (9.82 grams, 17.6 mmol) and sodium hydride (3.38 grams, 140 mmol) in DMF (200 mL), was added p-Methoxybenzyl chloride (14.3 mL, 105.3 mmol) at 0° C. The reaction progress was monitored by TLC (EtOAc/Hexane 3:7). After 8 hours the reaction was completed and ice was added in small portions to quench the reaction. The mixture was diluted with ethyl acetate (100 mL) and washed with water (2×50 mL). The combined aqueous layers were extracted with diethyl ether (2×50 mL); the combined organic layers were dried over anhydrous MgSO₄, and evaporated to dryness. The residue was purified by column chromatography (EtOAc/Hexane 8:92) to thereby obtain compound 19 (15.28 grams, 84%).

¹H NMR (500 MHz, CDCl₃): ‘Ring I’: δ=H 5.45 (d, 1H, J=3.5 Hz, H-1), 3.94 (m, 2H, H-3, H-5), 3.88-3.78 (m, 2H, H-6), 3.59 (t, 1H, J=9.5 Hz, H-4), 3.17 (dd, 1H, J1=10.5, J2=3.5 Hz, H-2); ‘Ring II’: δ=H 3.56-3.42 (m, 2H, H-4, H-5), 3.41-3.32 (m, 1H, H-1), 3.32-3.20 (m, 2H, H-3, H-6), 2.17 (dt, 1H, J1=12.5, J2=4.0 Hz, H-2eq), 1.34 (ddd, 1H, J1=J2=J3=12.5 Hz, H-2ax); The additional peaks in the spectrum were identified as follow: δ=H 7.21 (d, 2H, J=8.0 Hz, PMB), 7.17 (d, 6H, J=8.0 Hz, PMB), 6.85-6.72 (m, 8H, PMB), 4.86 (d, 1H, J=10.0 Hz, PMB), 4.80-4.65 (m, 6H, PMB), 4.61 (d, 1H, J=10.0 Hz, PMB), 3.74-3.68 (m, 12H, PMB), 1.04-0.94 (m, 21H, TIPS).

¹³C NMR (125 MHz, CDCl₃): δ=C 159.5 (PMB), 159.4 (PMB), 159.3 (PMB), 159.2 (PMB), 130.7 (PMB), 130.3 (PMB), 130.2 (PMB), 129.9 (PMB), 129.8 (PMB), 129.7 (PMB), 129.3 (PMB), 128.7 (PMB), 113.9 (2C, PMB), 97.5 (C1′), 84.5, 84.4, 79.8, 77.9 (C4′), 76.9, 75.6 (PMB), 75.2 (PMB), 74.9 (PMB), 74.5 (PMB), 72.9, 63.5 (C2′), 62.3 (C6′), 60.3 (C1), 59.5, 55.3 (4C, PMB), 32.4 (C2), 18.1 (2C, TIPS), 12.1 (TIPS).

MALDI TOFMS: calculated for C₅₃H₇₁N₉O₁₁Si ([M+Na]+) m/e 1061.2; measured m/e 1061.6.

### Synthesis of (2R,3R,4R,5R,6R)-3-azido-2-(((1R,2R,3S,4R,6S)-4,6-diazido-2,3-bis((4-methoxybenzyl)oxy)cyclohexyl)oxy)-4,5-bis((4-methoxybenzyl) oxy)-6-vinyltetrahydro-2H-pyran (Compound 20)

To a stirred solution of compound 19 (19.82 grams, 19 mmol) in THF (230 mL) at 0° C., TBAF (11.05 mL, 38.1 mmol) was added and the reaction progress was monitored by TLC (EtOAc/Hexane 2:3). After 19 hours, the solvent was evaporated to dryness and the obtained residue was subjected to column chromatography (EtOAc/Hexane 3:7) to thereby obtain the corresponding 6′-alcohol (14.74 grams, 88%).

¹H NMR (500 MHz, CDCl₃): ‘Ring I’: δ=H 5.51 (d, 1H, J=4.0 Hz, H-1), 3.98 (dt, 1H, J1=8.0, J2=2.0 Hz, H-5), 3.92 (t, 1H, J=10.0 Hz, H-3), 3.70 (dd, 1H, J1=12.0, J2=2.0 Hz, H-6), 3.64 (dd, 1H, J1=12.0, J2=2.0 Hz, H-6), 3.49 (dd, 1H, J1=10.0, J2=8.0 Hz, H-4), 3.17 (dd, 1H, J1=10.0, J2=4.0 Hz, H-2); ‘Ring II’: δ=H 3.53-3.44 (m, 2H, H-4, H-5), 3.38 (ddd, 1H, J1=12.5, J2=10.0, J3=4.5 Hz, H-1), 3.34-3.24 (m, 2H, H-3, H-6), 2.20 (dt, 1H, J1=12.5, J2=4.5 Hz, H-2eq), 1.36 (ddd, 1H, J1=J2=J3=12.5 Hz, H-2ax); The additional peaks in the spectrum were identified as follow: δ=H 7.23 (d, 2H, J=8.0 Hz, PMB), 7.20-7.14 (m, 6H, PMB), 6.83-6.75 (m, 8H, PMB), 4.89 (d, 1H, J=10.0 Hz, PMB), 4.80-4.68 (m, 6H, PMB), 4.55 (d, 1H, J=10.0 Hz, PMB), 3.73-3.7 (m, 12H, PMB).

¹³C NMR (125 MHz, CDCl₃): δ=C 159.5 (PMB), 159.4 (2C, PMB), 159.2 (PMB), 130.2 (PMB), 130.1 (2C, PMB), 129.9 (PMB), 129.8 (PMB), 129.6 (2C, PMB), 128.8 (PMB), 114.0 (2C, PMB), 113.9 (2C, PMB), 97.6 (C1′), 84.4, 84.3, 79.8 (C3′), 77.4, 75.6 (PMB), 75.2 (PMB), 75.1 (PMB), 74.6 (PMB), 72.0 (C5′), 63.3 (C2′), 61.4 (C6′), 60.3 (C1), 59.5, 55.3 (3C, PMB), 32.4 (C2).

MALDI TOFMS: calculated for C44H₅₁N₉O₁₁ ([M+Na]+) m/e 903.3; measured m/e 903.9.

To a solution of the 6′-alcohol from the above reaction (100 mg, 0.11 mmol) in ethyl acetate (5 mL), IBX (95 mg, 0.33 mmol) was added in one portion. The resulting suspension was heated at 80° C. and stirred vigorously. After the reaction was completed (3.5 hours) as indicated by TLC (EtOAc/Hexane 2:3), the reaction was cooled to room temperature and filtered through Celite®. The Celite® was thoroughly washed with ethyl acetate (2×50 mL) and the combined organic layers were evaporated under reduced pressure. The crude product was subjected to flash column chromatography (EtOAc/Hexane 35:65) to thereby obtain the 6′-aldehyde (85 mg, 85%).

¹H NMR (500 MHz, CDCl₃): ‘Ring I’: δ=H 9.53 (s, 1H, H-6), 5.56 (d, 1H, J=4.0 Hz, H-1), 4.60 (d, 1H, J=10.0 Hz, H-4), 3.98 (dd, 1H, J1=J2=10.0 Hz, H-3), 3.52-3.45 (m, 1H, H-5), 3.17 (dd, 1H, J1=10.0, J2=4.0 Hz, H-2); ‘Ring II’: δ=H 3.53-3.43 (m, 2H, H-4, H-5), 3.37 (ddd, 1H, J1=12.0, J2=10.0, J3=4.0 Hz, H-1), 3.33-3.24 (m, 2H, H-3, H-6), 2.20 (dt, 1H, J1=12.5, J2=4.0 Hz, H-2eq), 1.35 (ddd, 1H, J1=J2=J3=12.5 Hz, H-2ax); The additional peaks in the spectrum were identified as follow: δ=H 7.23 (d, 2H, J=8.0 Hz, PMB), 7.19-7.10 (m, 6H, PMB), 6.83-6.72 (m, 8H, PMB), 4.89 (d, 1H, J=10.0 Hz, PMB), 4.80-4.64 (m, 6H, PMB), 4.51 (d, 1H, J=10.0 Hz, PMB), 3.73 (s, 3H, PMB), 3.71 (s, 6H, PMB), 3.70 (s, 3H, PMB).

¹³C NMR (125 MHz, CDCl₃): δ=C 197.3 (CHO), 159.7 (PMB), 159.6 (2C, PMB), 159.2 (PMB), 130.2 (PMB), 130.0 (PMB), 129.9 (2C, PMB), 129.7 (PMB), 129.6 (PMB), 129.3 (PMB), 128.6 (PMB), 114.1 (PMB), 114.0 (3C, PMB), 97.5 (C1′), 84.3, 84.2, 79.8 (C3′), 78.0, 77.6, 75.6 (PMB), 75.5 (PMB), 75.2 (C4′), 75.1 (PMB), 74.8 (PMB), 62.8 (C2′), 60.2 (C1), 59.1, 55.4 (PMB), 55.3 (PMB), 32.21 (C2).

MALDI TOFMS: calculated for C44H₄₉N₉O₁₁ ([M+Na]+) m/e 902.3; measured m/e 902.3.

To a cooled suspension of Methyltriphenylphosphonium Iodide (70 mg, 0.19 mmol) in anhydrous THF at 0° C., n-BuLi (1.6 M in hexane, 136 μL) was added drop wise and the resulting yellow solution was stirred for additional 30 minutes at 0° C. The 6′-aldehyde from the previous step (61 mg, 0.069 mmol) in anhydrous THF (0.3 mL) was thereafter added at 0° C., and the reaction was allowed to stir for additional 1.5 hours at room temperature. After completion of the reaction, as indicated by TLC (EtOAc/Hexane 2:3), the reaction was quenched with saturated NH₄Cl solution. The layers were separated and the aqueous layer was extracted with ether (2×10 mL). The combined organic layers were washed with brine, dried over anhydrous MgSO₄ and evaporated to dryness. The crude product was purified by flash chromatography (EtOAc/Hexane 2.5:7.5) to thereby obtain Compound 20 (27 mg, 56%).

¹H NMR (500 MHz, CDCl₃): ‘Ring I’: δ=H 5.83-5.74 (m, 1H, H-6), 5.47 (d, 1H, J=4.0 Hz, H-1), 5.37 (d, 1H, J=16.5 Hz, H-7trans), 5.21 (d, 1H, J=9.5 Hz, H-7cis), 4.49 (dd, 1H, J1=9.5, J2=7.5 Hz, H-5), 3.90 (t, 1H, J=9.5 Hz, H-3), 3.25-3.14 (m, 2H, H-2, H-4); ‘Ring II’: δ=H 3.54-3.44 (m, 2H, H-4, H-5), 3.38 (ddd, 1H, J1=12.0, J2=9.5, J3=4.0 Hz, H-1), 3.34-3.25 (m, 2H, H-3, H-6), 2.21 (dt, 1H, J1=12.5, J2=4.0 Hz, H-2eq), 1.38 (ddd, 1H, J1=J2=J3=12.5 Hz, H-2ax); The additional peaks in the spectrum were identified as follow: δ=H 7.25-7.09 (m, 8H, PMB), 6.84-6.73 (m, 8H, PMB), 4.88 (d, 1H, J=10.0 Hz, PMB), 4.77 (dd, 2H, J=10.0, 2.5 Hz, PMB), 4.74-4.66 (m, 3H, PMB), 4.59 (d, 1H, J=10.5 Hz, PMB), 4.52 (d, 1H, J=10.5 Hz, PMB), 3.73 (s, 3H, PMB), 3.72 (s, 6H, PMB), 3.71 (s, 3H, PMB).

¹³C NMR (125 MHz, CDCl3): δ=C 159.5 (PMB), 159.4 (2C, PMB), 159.2 (PMB), 134.9 (C6′), 130.3 (PMB), 130.2 (2C, PMB), 129.9 (2C, PMB), 129.6 (2C, PMB), 128.7 (PMB), 118.8 (C7′), 114.0 (PMB), 113.9 (2C, PMB), 97.6 (C1′), 84.4, 84.3, 82.4 (C4′), 79.4 (C3′), 77.6, 75.6 (PMB), 75.3 (PMB), 75.0 (PMB), 74.6 (PMB), 72.7 (C5′), 63.4 (C2′), 60.3 (C1), 59.3, 55.4 (PMB), 55.3 (PMB), 32.3 (C2).

MALDI TOFMS: calculated for C45H51N9O10 ([M+Na]+) m/e 900.9; measured m/e 900.5.

### Synthesis of 1-((2R,3S,4R,5R,6S)-5-azido-6-(((1R,2R,3S,4R,6S)-4,6-diazido-2,3-bis((4-methoxybenzyl)oxy)cyclohexyl)oxy)-3,4-bis((4-methoxybenzyl) oxy)tetrahydro-2H-pyran-2-yl)ethane-1,2-diol (Compound 21)

To a stirred solution of Compound 20 (383 mg, 0.436 mmol) in acetone (5 mL), water (1.5 mL) and t-BuOH (5 mL), K₂OsO₄.2H2O (16 mg, 0.043 mmol) and NMO (181 μL) were added sequentially. The progress of the reaction was monitored by TLC (EtOAc/Hexane 2:3), which indicated the completion after 24 hours. The solvent was then evaporated to dryness; the residue was dissolved in EtOAc to which an aqueous solution of Na₂S₂O₃ was added. The layers were separated and the organic phase was washed with brine, dried over MgSO₄ and evaporated. The crude product was subjected to column chromatography (EtOAc/Hexane 1:1) to thereby obtain compound 21 (370 mg, 93%) as a 6′-diasteromeric mixture.

### Synthesis of (2R,3S,4R,5R,6S)-5-azido-6-(((1R,2R,3S,4R,6S)-4,6-diazido-2,3-dihydroxycyclohexyl)oxy)-2-((R)-1,2-dihydroxyethyl)tetrahydro-2H-pyran-3,4-diol (Compound 22) and (2R,3S,4R,5R,6S)-5-azido-6-(((1R,2R,3S,4R,6S)-4,6-diazido-2,3-dihydroxycyclohexyl)oxy)-2-((S)-1,2-dihydroxyethyl) tetrahydro-2H-pyran-3,4-diol (Compound 23)

Compound 21 (220 mg, 1.0 equiv.) from above was stirred with 2,3-dichloro-5,6-dicyano-1,4-benzoquinone (DDQ) (383 mg, 6 equiv.) in methylene chloride and water (20:1 v/v, 15 mL) at room temperature. After the addition of DDQ, a dark green color charge transfer complex formed immediately and slowly faded to orange color as the reaction progressed. TLC (EtOAc/MeOH 98:2) showed that the reaction completed after 15 hours. The solvents were then evaporated and the residue was loaded onto the silica gel column without prior work up. Due to high polarity of the titled compound, this column chromatography allowed the removal of only parts of the DDQ reaction byproducts. Therefore, in order to obtain the analytically pure product, the fractions containing the product were combined, evaporated and the residue was then subjected to peracetylation and deacetylation steps, as follows. The crude material from above was dissolved in anhydrous pyridine (5 mL) and cooled to 0° C. Acetic anhydride (0.73 mL, 9 equiv.) was added dropwise, followed by the addition of 4-DMAP (0.621 gram, 6 equiv.). After completion of the reaction (2 hours), as indicated by TLC (EtOAc/Hexane 2:3), the reaction was diluted with EtOAc (20 mL) and washed with 5% HCl solution, Sat. NaHCO₃, and brine, and dried over anhydrous MgSO₄. The solvent was evaporated to dryness and the residue was subjected to a column chromatography (EtOAc/Hexane 3:7) to thereby obtain the corresponding peracetate as an inseparable mixture of 6′-diastereomers (150 mg, 91% for 2 steps).

The peracetate (215 mg, 0.314 mmol) from above was dissolved in anhydrous MeOH (5 mL) and NaOMe (152 mg, 2.81 mmol) was added in one portion to the stirred solution at room temperature. The reaction progress was monitored by TLC (EtOAc/MeOH 95:5), which indicated completion after 4 hours. The reaction mixture was passed through a short silica gel column and the product was eluted with MeOH. The fractions with the compound were combined, evaporated and the crude product was subjected to an additional column chromatography (EtOAc/MeOH 99:1), which allowed complete separation of the two diastereomers, the major (Rf=0.36) and minor (Rf=0.2). The major diastereomer was later assigned, as detailed hereinunder, as the 6′-(R)-diastereomer (Compound 22) and the minor one as the 6′-(S)-diastereomer (Compound 23).

Major Diastereomer (22): ¹H NMR (500 MHz, MeOD): ‘Ring I’: δ=H 5.68 (d, 1H, J=4.0 Hz, H-1), 4.04 (dd, 1H, J1=9.5, J2=4.0 Hz, H-4), 3.97-3.92 (m, 1H, H-6), 3.93 (t, 1H, J=10.0 Hz, H-3), 3.79 (dd, 1H, J=11.5, 3.5 Hz, H-7), 3.70 (dd, 1H, J1=11.5, J2=7.0 Hz, H-7), 3.58 (t, 1H, J=9.5 Hz, H-5), 3.13 (dd, 1H, J1=10.0, J2=4.0 Hz, H-2); ‘Ring II’: δ=H 3.57-3.47 (m, 3H, H-3, H-4, H-5), 3.44 (ddd, 1H, J1=16.5, J2=8.5, J3=4.0 Hz, H-1), 3.29 (t, 1H, J=9.5 Hz, H-6), 2.26 (dt, 1H, J1=12.5, J2=4.0 Hz, H-2eq), 1.43 (ddd, 1H, J1=J2=J3=12.5 Hz, H-2ax).

¹³C NMR (125 MHz, MeOD): δ=C 99.1 (C1′), 80.5, 77.9 (C6), 77.9, 74.8 (C6′), 73.7 (C4′), 72.9 (C5′), 72.3 (C3′), 64.5 (C2′), 64.3 (C7′), 61.7 (C1), 61.0, 33.3 (C2).

MALDI TOFMS: calculated for C13H₂₇N₃O₈ ([M+H]+) m/e 432.3; measured m/e 432.8.

Minor Diastereomer (23): ¹H NMR (500 MHz, MeOD): Ring I′: δ=H 5.72 (d, 1H, J=3.6 Hz, H-1), 4.00 (ddd, 1H, J1=6.8, J2=6.0, J3=1.1 Hz, H-6), 3.97-3.91 (m, 2H, H-5, H-3), 3.74-3.67 (m, 2H, H-7, H-7), 3.64-3.59 (m, 1H, H-4), 3.10 (dd, 1H, J1=10.5, J2=3.7 Hz, H-1); ‘Ring II’: δ=H 3.57-3.50 (m, 2H, H-1, H-6), 3.45-3.37 (m, 2H, H-3, H-4), 3.26 (t, 1H, J=9.5 Hz, H-5), 2.24 (dt, 1H, J1=12.8, J2=4.4 Hz, H-2eq), 1.40 (ddd, 1H, J1=J2=J3=12.5 Hz, H-2ax).

¹³C NMR (125 MHz, MeOD): δ=C 99.1 (C-1′), 80.1 (C-4), 78.0 (C-6), 77.8 (C-5), 73.1 (C-5′), 72.3 (C-3′), 71.3 (C-4′), 70.8 (C-6′), 65.3 (C-7′), 64.4 (C-2′), 61.7 (C-3), 61.1 (C-1), 33.3 (C-2).

MALDI TOFMS: calculated for C13H27N3O8 ([M+H]+) m/e 432.3; measured m/e 432.8.

### Synthesis of (2R,3S,4R,5R,6S)-5-amino-6-(((1R,2R,3S,4R,6S)-4,6-diamino-2,3-dihydroxycyclohexyl)oxy)-2-((R)-1,2-dihydroxyethyl)tetrahydro-2H-pyran-3,4-diol [NB153 ((R)-diasteromer)]

To a stirred solution of compound 22 (82 mg, 0.19 mmol) in a mixture of THF (3 mL) and aqueous NaOH (1 mM, 5 mL), PMe₃ (1 M solution in THF, 0.15 mL, 2.5 mmol) was added. The progress of the reaction was monitored by TLC [CH₂Cl₂/MeOH/H₂O/MeNH₂ (33% solution in EtOH), 10:15:6:15], which indicated completion after 1 hour. The reaction mixture was thereafter purified by flash chromatography on a short column of silica gel. The column was washed with the following solvents: THF (100 mL), CH₂Cl₂ (100 mL), EtOH (50 mL), and MeOH (100 mL). The product was then eluted with a mixture of 5% MeNH₂ solution (33% solution in EtOH) in 80% MeOH. Fractions containing the product were combined and evaporated under vacuum. The pure product was obtained by passing the above product through a short column of Amberlite CG50 (NH4⁺ form). First, the column was washed with water, then the product was eluted with a mixture of 10% NH₄OH in water, to thereby obtain NB153 (49.0 mg, 73%). For storage and biological tests, NB153 was converted to its sulfate salt form as follow: The free base form was dissolved in water, the pH was adjusted to 6.7 with H₂SO₄ (0.1 N) and lyophilized to afford the sulfate salt of NB153 as white foamy solid.

¹H-NMR (500 MHz, MeOD, —NH2 form): ‘Ring I’: δ=H 5.18 (d, 1H, J=4.0 Hz, H-1), 3.98-3.93 (m, 1H, H-6), 3.90 (dd, 1H, J1=10.0, J2=4.0 Hz, H-4), 3.76 (dd, 1H, J1=11.5, J2=4.0 Hz, H-7), 3.70 (dd, 1H, J1=11.5, J2=6.0 Hz, H-7), 3.51 (t, 1H, J=10.0 Hz, H-3), 3.44 (m, 1H, H-5), 2.74 (dd, 1H, J1=10.0, J2=4.0 Hz, H-2); ‘Ring II’: δ=H 3.43 (t, 1H, J=9.0 Hz, H-5), 3.20 (t, 1H, J=9.0 Hz, H-4), 3.10 (t, 1H, J=9.5 Hz, H-6), 2.77 (ddd, 1H, J1=10.5, J2=9.0, J3=5.0 Hz, H-3), 2.66 (ddd, 1H, J1=10.5, J2=9.5, J3=5.0 Hz, H-1), 2.02 (dt, 1H, J1=12.5, J2=4.0 Hz, H-2eq), 1.22 (ddd, 1H, J1=J2=J3=12.5 Hz, H-2ax).

¹³C NMR (125 MHz, MeOD): δC 102.9 (C-1′), 90.0 (C-4), 78.2 (C-6), 77.5, 75.6 (C-3′), 74.3 (C-4′), 73.6 (C-6′), 73.3, 63.3 (C-7′), 57.1 (C-2′), 52.4 (C-3), 51.3 (C-1), 36.7 (C2).

MALDI TOFMS: calculated for C₁₃H₂₇N₃O₈ ([M+H]+) m/e 354.3; measured m/e 354.8.

### Synthesis of (2R,3S,4R,5R,6S)-5-amino-6-(((1R,2R,3S,4R,6S)-4,6-diamino-2,3-dihydroxycyclohexyl)oxy)-2-((S)-1,2-dihydroxyethyl)tetrahydro-2H-pyran-3,4-diol [NB155 ((S)-diastereomer)]

To a stirred solution of Compound 23 (52 mg, 0.12 mmol) in a mixture of THF (3 mL) and aqueous NaOH (1 mM, 5 mL), PMe₃ (1 M solution in THF, 0.15 mL, 2.5 mmol) was added. The progress of the reaction was monitored by TLC [CH₂Cl₂/MeOH/H₂O/MeNH₂ (33% solution in EtOH), 10:15:6:15], which indicated completion after 1 hour. The reaction mixture was purified by flash chromatography on a short column of silica gel. The column was washed with the following solvents: THF (100 mL), CH₂Cl₂ (100 mL), EtOH (50 mL), and MeOH (100 mL). The product was then eluted with the mixture of 5% MeNH₂ solution (33% solution in EtOH) in 80% MeOH. Fractions containing the product were combined and evaporated under vacuum. The pure product was obtained by passing the above product through a short column of Amberlite CG50 (NH4⁺ form). First, the column was washed with water, then the product was eluted with a mixture of 10% NH₄OH in water to thereby obtain NB155 (36.0 mg, 78%). For storage and biological tests, NB155 was converted to its sulfate salt form as follow: The free base form was dissolved in water, the pH was adjusted to 6.7 with H₂SO₄ (0.1 N) and lyophilized to afford the sulfate salt of NB155 as white foamy solid.

¹H NMR (500 MHz, MeOD, —NH₂ form): ‘Ring I’: δ=H 5.28 (d, 1H, J=3.8 Hz, H-1′), 3.97 (td, 1H, J1=7.1, J2=1.0 Hz, H-6′), 3.89-3.82 (m, 1H, H-4′), 3.63 (d, 2H, J=7.2 Hz, H-7′, H-7′), 3.59-3.51 (m, 2H, H-5′, H-3′), 2.72 (m, 1H, H-2′); ‘Ring II’: δ=H 3.41 (t, 1H, J=9.1 Hz, H-5), 3.20 (t, 1H, J=9.2 Hz, H-4), 3.08 (t, 1H, J=9.4 Hz, H-6), 2.74 (m, 1H, H-3), 2.64 (ddd, 1H, J1=12.2, J2=9.7, J3=4.1 Hz, H-1), 2.00 (dt, 1H, J1=12.9, J2=4.1 Hz, H-2eq), 1.21 (ddd, 1H, J1=J2=J3=12.3 Hz, H-2ax).

¹³C NMR (125 MHz, MeOD): δ=C 102.9 (C-1′), 89.6 (C-4), 79.0 (C-6), 77.9 (C-5), 75.8 (C-3′), 72.3 (C-4′), 71.1 (C-5′), 70.2 (C-6′), 63.2 (C-7′), 57.1 (C-2′), 52.4 (C-1), 51.4 (C-3), 37.7 (C-2).

MALDI TOFMS: calculated for C₁₃H₂₇N₃O₈ ([M+H]+) m/e 354.3; measured m/e 354.8.

### Syntheses of Pseudo-Trisaccharides NB156 and NB157

The syntheses of compounds NB156 and NB157 are illustrated in Scheme 2 below, and were accomplished from the intermediate Compound 22 by using essentially the same chemical transformations as for NB153 and NB155.

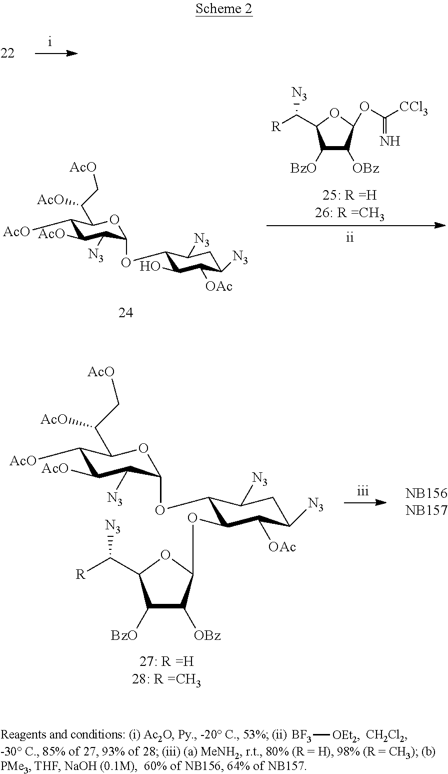

Briefly, regioselective acetylation of Compound 22 with Ac₂O at low temperature gave the corresponding C5 acceptor Compound 24. For the glycosylation of 24 the trichloroacemidate donors 25 and 26 which furnished the corresponding pseudo-trisaccharides 27 and 28 in 85% and 93% isolated yields, respectively, exclusively as 3-anomers. Treatment with methylamine was followed by Staudinger reaction to afford NB156 and NB157.

### Synthesis of NB156

### Synthesis of (2R,3S,4R,5R,6S)-6-(((1R,2S,3S,4R,6S)-3-acetoxy-4, 6-diazido-2-hydroxycyclohexyl)oxy)-5-azido-2-((R)-1,2-diacetoxyethyl)tetrahydro-2H-pyran-3,4-diyl diacetate (24)

Compound 22 (370 mg, 0.857 mmol) was dissolved in anhydrous pyridine (8 mL) and cooled to −20° C. Acetic anhydride (0.45 mL, 4.8 mmol) was added dropwise and the reaction was allowed to progress at −20° C. The reaction progress was monitored by TLC, which indicated completion after 17 hours. The reaction mixture was diluted with EtOAc, and extracted with aqueous solution of HCl (2%), saturated aqueous NaHCO₃, and brine. The combined organic layers were dried over anhydrous MgSO₄ and concentrated. The crude product was purified by silica gel column chromatography (EtOAc/Hexane 3:7) to afford Compound 24 (292 mg, 53% yield).

¹H NMR (500 MHz, CDCl₃): ‘Ring I’: δ=H 5.45 (dd, 1H, J1=10.5, J2=9.3 Hz, H-3′), 5.37 (d, 1H, J=3.5 Hz, H-1′), 5.19 (ddd, 1H, J1=7.6, J2=4.0, J3=2.0 Hz, H-6′), 5.07 (dd, 1H, J1=10.4, J2=9.2 Hz, H-4′), 4.40 (dd, 1H, J1=10.5, J2=1.8 Hz, H-5′), 4.31 (dd, 1H, J1=12.0, J2=4.1 Hz, H-7′), 4.19-4.08 (m, 1H, H-7′), 3.63-3.56 (m, 1H, H-2′); ‘Ring II’: δ=H 4.91 (dd, 1H, J1=12.8, J2=7.1 Hz, H-6) 3.66 (td, 1H, J1=9.6, J2=3.5 Hz, H-5), 3.53 (ddd, 1H, J1=12.4, J2=10.1, J3=4.5 Hz, H-1), 3.45 (dd, 1H, J1=19.1, J2=9.2 Hz, H-4), 3.38-3.31 (m, 1H, H-3), 2.38 (dt, 1H, J1=13.2, J2=4.4 Hz, H-2eq), 1.58 (ddd, 1H, J1=J2=J3=12.6 Hz, H-2ax). The additional peaks in the spectrum were identified as follow: δ=H 2.17 (s, 3H, CH₃CO), 2.08 (d, 9H, J=1.5 Hz, CH₃CO), 2.04 (s, 3H, CH₃CO).

¹³C NMR (125 MHz, CDCl₃): δ=C 170.7 (C═O), 170.6 (C═O), 170.2 (C═O), 170.0 (C═O), 169.9 (C═O), 98.5 (C-1′), 82.9 (C-4), 75.1 (C-6), 74.6 (C-5), 71.4 (C-3′), 70.0 (C-6′), 69.9 (C-5′), 68.9 (C-4′), 61.8 (C-7′), 61.5 (C-2′), 58.2 (C-3), 58.0 (C-1), 32.0 (C-2), 20.96 (CH₃), 20.92 (CH₃), 20.89 (CH₃), 20.86 (CH₃), 20.8 (CH₃), 20.7 (CH₃).

MALDI TOFMS: calculated for C₂₃H₃₁N₉O₁₃ ([M+Na]+) m/e 664.20; measured m/e 664.20.

### Synthesis of (2S,3S,4S,5R)-2-(((1S,2S,3R,5S,6R)-2-acetoxy-3,5-diazido-6-(((2S,3R,4R,5S,6R)-4,5-diacetoxy-3-azido-6-((R)-1,2-diacetoxyethyl)tetrahydro-2H-pyran-2-yl)oxy)cyclohexyl)oxy)-5-(azidomethyl)tetrahydrofuran-3,4-diyl dibenzoate (27)

Anhydrous CH₂Cl₂ (15 mL) was added to a powdered, flame-dried 4 Å molecular sieves (2.0 grams), followed by the addition of acceptor Compound 24 (292 mg, 0.455 mmol) and donor Compound 25 (1.0 gram, 1.9 mmol). The reaction mixture was stirred for 10 minutes at room temperature and was then cooled to −30° C. Catalytic amount of BF₃-Et₂O (50 μL) was added and the mixture was stirred at −30° C.; the reaction progress was monitored by TLC, which indicated the completion after 60 minutes. The reaction mixture was diluted with ethyl acetate and washed with saturated NaHCO₃ and brine. The combined organic layer was dried over MgSO₄, evaporated and subjected to column chromatography (EtOAc/Hexane) to obtain the Compound 27 (393 mg, 85% yield).

¹H NMR (500 MHz, CDCl₃): ‘Ring I’: δ=H 5.87 (d, 1H, J=3.8 Hz, H-1), 5.42-5.34 (m, 1H, H-3), 5.24-5.13 (m, 1H, H-6), 5.10-5.03 (m, 1H, H-4), 4.54 (dd, 1H, J1=10.5, J2=2.2 Hz, H-5), 4.33 (dd, 1H, J1=12.0, J2=4.1 Hz, H-7), 4.20 (dd, 1H, J1=11.9, J2=7.8 Hz, H-7), 3.50 (dd, 1H, J1=10.9, J2=3.8 Hz, H-2); ‘Ring II’: δ=H 5.01 (t, 1H, J=10.0 Hz, H-6), 3.87 (t, 1H, J=9.3 Hz, H-5), 3.71 (t, 1H, J=9.5 Hz, H-4), 3.57-3.48 (m, 2H, H-1, H-3), 2.38 (dt, 1H, J1=12.9, J2=4.3 Hz, H-2eq), 1.52 (ddd, 1H, J1=J2=J3=12.7 Hz, H-2ax); ‘Ring III’: δ=H 5.61 (s, 1H, H-1), 5.57 (d, 1H, J=4.7 Hz, H-2), 5.42-5.35 (m, 1H, H-3), 4.59-4.47 (m, 1H, H-4), 3.63-3.55 (m, 2H, H-5, H-5). The additional peaks in the spectrum were identified as follow: δ=H 7.93 (d, 2H, J=7.1 Hz, Ar), 7.87 (d, 2H, J=7.1 Hz, Ar), 7.54 (dt, 2H, J1=19.0, J2=7.4 Hz, Ar), 7.39 (t, 2H, J=7.8 Hz, Ar), 7.34 (t, 2H, J=7.8 Hz, Ar), 2.29 (s, 3H, CH₃), 2.08-2.04 (m, 12H, 4×CH₃).

¹³C NMR (125 MHz, CDCl₃): δ=C 170.7 (C═O), 170.1 (C═O), 170.08 (C═O), 170.06 (C═O), 169.9 (C═O), 165.5 (Ar), 165.2 (Ar), 133.8 (Ar), 133.7 (Ar), 129.8 (Ar), 129.7 (Ar), 128.8 (Ar), 128.68 (Ar), 128.63 (Ar), 128.5 (Ar), 107.7 (C-1″), 96.1 (C-1′), 80.9 (C-4″), 79.8 (C-5), 77.2 (C-4), 74.5 (C-2″), 73.9 (C-6), 72.0 (C-3′), 70.7 (C-3″), 69.9 (C-6′), 69.2 (C-5′), 68.8 (C-4′), 61.5 (C-7′), 61.4 (C-2′), 58.9 (C-3), 58.3 (C-1), 53.1 (C-5″), 32.1 (C-2), 21.04 (CH₃), 21.03 (CH₃), 20.8 (CH₃), 20.7 (CH₃), 20.6 (CH₃).

MALDI TOFMS: calculated for C₄₂H₄₆N₁₂O₁₈ ([M+Na]+) m/e 1029.31; measured m/e 1029.29.

### Synthesis of (2R,3S,4R,5R,6S)-5-amino-6-(((1R,2R,3S,4R,6S)-4,6-diamino-2-(((2S,3S,4R,5R)-5-(aminomethyl)-3,4-dihydroxytetrahydrofuran-2-yl)oxy)-3-hydroxycyclohexyl)oxy)-2-((R)-1,2-dihydroxyethyl)tetrahydro-2H-pyran-3,4-diol (NB156)

The glycosylation product 27 (393 mg, 0.390 mmol) was treated with a solution of MeNH₂ (33% solution in EtOH, 15 mL) and the reaction progress was monitored by TLC (EtOAc/MeOH 85:15), which indicated completion after 12 hours. The reaction mixture was evaporated to dryness and was subjected to column chromatography (MeOH/EtOAc 2:8) to thereby obtain the corresponding completely de-esterified perazido derivative (183 mg) in 80% yield.

¹H NMR (500 MHz, MeOD): ‘Ring I’: δ=H 5.89 (d, 1H, J=3.8 Hz, H-1), 3.97 (dd, 1H, J1=9.7, J2=4.6 Hz, H-5), 3.84 (dd, 2H, J1=12.0, J2=7.1 Hz, H-6, H-3), 3.69 (d, 1H, J=8.5 Hz, H-7), 3.60 (dd, 1H, J1=11.6, J2=6.4 Hz, H-7), 3.45 (dd, 1H, J1=10.0, J2=8.7 Hz, H-4), 3.06 (dd, 1H, J1=10.6, J2=4.4 Hz, H-2); ‘Ring II’: δ=H 3.62-3.54 (m, 2H, H-4, H-5), 3.50-3.43 (m, 1H, H-3), 3.40-3.33 (m, 1H, H-1), 3.33-3.26 (m, 1H, H-6), 2.12 (dt, 1H, J1=13.3, J2=4.4 Hz, H-2 eq), 1.29 (ddd, 1H, J1=J2=J3=12.4 Hz, H-2 ax); ‘Ring III’: δ=H 5.28 (d, 1H, J=0.8 Hz, H-1), 4.11 (dd, 1H, J1=4.4, J2=0.8 Hz, H-2), 3.98 (dd, 1H, J1=7.4, J2=4.2 Hz, H-3), 3.94 (dd, 1H, J1=7.0, J2=3.4 Hz, H-4), 3.49 (dd, 1H, J1=13.3, J2=2.8 Hz, H-5), 3.41 (dd, 1H, J1=13.1, J2=6.3 Hz, H-5).

¹³C NMR (125 MHz, MeOD): δ=C 111.2 (C-1″), 97.4 (C-1′), 85.2 (C-4), 82.3 (C-5′), 77.6 (C-6), 76.8 (C-5), 76.3 (C-2″), 74.6 (C-6′), 73.3 (C-3″), 73.2 (C-4′), 72.7 (C-4″), 72.5 (C-3′), 64.7 (C-2′), 64.1 (C-7′), 61.9 (C-3), 61.4 (C-1), 54.5 (C-5″), 33.1 (C-2).

MALDI TOFMS: calculated for C₁₈H₂N₁₂O₁₁ ([M+Na]+) m/e 611.20; measured m/e 611.19.

To a stirred solution of a perazido derivative from the above reaction (183 mg, 0.311 mmol) in a mixture of THF (3 mL) and aqueous NaOH (1 mM, 5 mL), PMe₃ (1 M solution in THF, 0.22 mL, 3.0 mmol) was added. The progress of the reaction was monitored by TLC [CH₂C12/MeOH/H₂O/MeNH₂ (33% solution in EtOH), 10:15:6:15], which indicated completion after 1 hour. The reaction mixture was purified by flash chromatography on a short column of silica gel. The column was washed with the following solvents: THF (100 mL), CH₂Cl₂ (100 mL), EtOH (50 mL), and MeOH (100 mL). The product was then eluted with the mixture of 5% MeNH₂ solution (33% solution in EtOH) in 80% MeOH. Fractions containing the product were combined and evaporated under vacuum. The pure product was obtained by passing the above product through a short column of Amberlite CG50 (NH4⁺ form). First, the column was washed with water, and then the product was eluted with a mixture of 10% NH₄OH in water to yield Compound NB156 as a free base form (90.0 mg, 60%).

For storage and biological tests, NB156 was converted to its sulfate salt form as follow: The free base form was dissolved in water, the pH was adjusted to 6.7 with H₂SO₄ (0.1 N) and lyophilized to afford the sulfate salt of NB156 as white foamy solid.

¹H NMR (500 MHz, MeOD): ‘Ring I’: δ=H 5.18 (d, 1H, J=3.6 Hz, H-1), 3.91 (dt, 1H, J1=6.3, J2=3.9 Hz, H-6), 3.85 (dd, 1H, J1=10.2, J2=2.8 Hz, H-5), 3.70 (dd, 1H, J1=11.5, J2=3.7 Hz, H-7), 3.64 (dd, 1H, J1=11.5, J2=6.4 Hz, H7), 3.50 (dd, 1H, J1=10.0, J2=9.0 Hz, H-3), 3.40 (t, 1H, J=9.5 Hz, H-4), 2.60 (dd, 1H, J=10.2, 3.3 Hz, H-2); ‘Ring II’: δ=H 3.44 (t, 1H, J=9.2 Hz, H-5), 3.33 (dd, 1H, J1=11.0, J2=7.6 Hz, H-4), 3.13 (t, 1H, J=9.5 Hz, H-6), 2.79-2.70 (m, 1H, H-3), 2.60 (td, 1H, J1=9.4, J2=4.4 Hz, H-1), 1.93 (dt, 1H, J1=13.0, J2=4.0 Hz, H-2eq), 1.16 (ddd, 1H, J1=J2=J3=12.4 Hz, H-2ax); ‘Ring III’: δ=H 5.20 (d, 1H, J=2.7 Hz, H-1), 4.04 (dd, 1H, J1=5.1, J2=2.8 Hz, H-2), 3.95-3.90 (m, 1H, H-3), 3.83 (dt, 1H, J1=5.3, J2=3.4 Hz, H-4), 2.89 (dd, 1H, J1=13.2, J2=4.0 Hz, H-5), 2.75 (dd, 1H, J1=13.2, J2=7.3 Hz, H-5).

¹³C NMR (125 MHz, MeOD): δ=C 110.6 (C-1″), 101.7 (C-1′), 86.8 (C-4), 85.5 (C-5), 84.7 (C-4″), 78.8 (C-6), 76.2 (C-2″), 75.3 (C-3′), 74.7 (C-5′), 73.8 (C-6′), 73.0 (C-4′), 72.5 (C-3″), 63.4 (C-7′), 57.5 (C-2′), 52.5 (C-3), 52.3 (C-1), 45.2 (C-5″), 37.5 (C-2).

MALDI TOFMS: calculated for C₁₈H₃₆N₄O₁₁ ([M+H]+) m/e 485.24; measured m/e 485.19.

### Synthesis of NB157

### Synthesis of (2S,3S,4S,5R)-2-(((1S,2S 3R, 5S, 6R)-2-acetoxy-3,5-diazido-6-(((2S,3R,4R,5S,6R)-4,5-diacetoxy-3-azido-6-((R)-1,2-diacetoxyethyl)tetrahydro-2H-pyran-2-yl)oxy)cyclohexyl)oxy)-5-((S)-1-azidoethyl)tetrahydrofuran-3,4-diyl dibenzoate (Compound 28)

Anhydrous CH₂Cl₂ (15 mL) was added to a powdered, flame-dried 4 Å molecular sieves (2.0 grams), followed by the addition of acceptor Compound 24 (265 mg, 0.413 mmol) and donor Compound 26 (0.895 gram, 1.65 mmol). The reaction mixture was stirred for 10 minutes at room temperature and was then cooled to −30° C. At this temperature, catalytic amount of BF₃-Et₂O (50 μL) was added and the mixture was stirred at −30° C. The reaction progress was monitored by TLC, which indicated the completion after 60 minutes. The reaction mixture was diluted with ethyl acetate and washed with saturated NaHCO₃ and brine. The combined organic layer was dried over MgSO₄, evaporated and subjected to column chromatography (EtOAc/Hexane) to obtain Compound 28 (393 mg) in 93% yield.

¹H NMR (600 MHz, CDCl₃): ‘Ring I’: δ=H 5.88 (d, 1H, J=4.0 Hz, H-1), 3.58 (dd, 1H, J1=10.7, J2=4.0 Hz, H-2), 5.36 (dd, 1H, J1=10.6, J2=9.3 Hz, H-3), 5.07 (dd, 1H, J1=10.5, J2=9.3 Hz, H-4), 4.53 (dd, 1H, J1=10.6, J2=2.2 Hz, H-5), 5.18 (ddd, 1H, J1=7.5, J2=4.1, J3=2.2 Hz, H-6), 4.33 (dd, 1H, J1=12.0, J2=3.9 Hz, H-7), 4.19 (dd, 1H, J1=12.1, J2=7.6 Hz, H-7); ‘Ring II’: δ=H 5.01 (t, 1H, J=9.9 Hz, H-6), 3.84 (t, 1H, J=9.4 Hz, H-5), 3.71 (t, 1H, J=9.5 Hz, H-4), 3.52 (ddd, 2H, J1=12.5, J2=10.0, J3=4.6 Hz, H-1, H-3), 2.39 (dt, 1H, J1=5.2, J2=4.5 Hz, H-2eq), 1.52 (ddd, 1H, J1=J2=J3=12.7 Hz, H-2ax); ‘Ring III’: δ=H 5.60 (t, 2H, J=2.3 Hz, H-1, H-2), 5.41 (dd, 1H, J1=7.6, J2=4.9 Hz, H-3), 4.33 (t, 1H, J=7.3 Hz, H-4), 3.77-3.64 (m, 1H, H-5), 1.24 (d, 3H, J=6.8 Hz, 6-CH3). The additional peaks in the spectrum were identified as follow: δ=H 7.92-7.89 (m, 2H, Ar), 7.89-7.85 (m, 2H, Ar), 7.60-7.50 (m, 2H, Ar), 7.39 (t, 2H, J=7.8 Hz, Ar), 7.34 (t, 2H, J=7.9 Hz, Ar), 2.41-2.35 (m, 3H, CH₃), 2.08 (s, 3H, CH₃), 2.07 (s, 3H, CH₃), 2.07 (s, 3H, CH₃), 2.05 (s, 3H, CH₃).

¹³C NMR (151 MHz, CDCl₃): δ=C 170.7 (C═O), 170.3 (C═O), 170.07 (C═O), 170.03 (C═O), 169.9 (C═O), 165.5 (Ar), 165.0 (Ar), 133.8 (Ar), 133.7 (Ar), 129.8 (Ar), 129.7 (Ar), 128.8 (Ar), 128.6 (Ar), 128.58 (Ar), 128.56 (Ar), 107.8 (C-1″), 96.1 (C-1′), 84.6 (C-4″), 79.7 (C-5), 77.6 (C-4), 74.7 (C-2″), 73.7 (C-6), 72.0 (C-3″), 71.0 (C-3), 70.0 (C-6′), 69.2 (C-4′), 68.9 (C-5′), 61.7 (C-2′), 61.5 (C-7′), 59.6 (C-5″), 58.9 (C-1), 58.5 (C-3), 32.2 (C-2), 21.1 (CH₃), 21.0 (CH₃), 20.8 (CH₃), 20.79 (CH₃), 20.78 (CH₃), 15.8 (C-6″, CH₃).

MALDI TOFMS: calculated for C₄₃H₄₈N₁₂O₁₈ ([M+Na]+) m/e 1043.32; measured m/e 1043.30.

### Synthesis of (2R,3S,4R,5R,6S)-5-amino-6-(((1R,2R,3S,4R,6S)-4,6-diamino-2-(((2S,3S,4R,5R)-5-((S)-1-aminoethyl)-3,4-dihydroxytetrahydrofuran-2-yl)oxy)-3-hydroxycyclohexyl)oxy)-2-((R)-1,2-dihydroxyethyl)tetrahydro-2H-pyran-3,4-diol (NB157)

The glycosylation product Compound 28 (0.393 gram, 0.384 mmol) was treated with a solution of MeNH₂ (33% solution in EtOH, 15 mL) and the reaction progress was monitored by TLC (EtOAc/MeOH 85:15), which indicated completion after 12 hours. The reaction mixture was evaporated to dryness and was subjected to column chromatography (MeOH/EtOAc 2:8) to obtain the corresponding completely de-esterified perazido derivative (230 mg) in 98% yield.

¹H NMR (600 MHz, MeOD): ‘Ring I’: δ=H 5.98 (d, 1H, J=3.8 Hz, H-1), 3.11 (dd, 1H, J1=10.5, J2=3.8 Hz, H-2), 4.03 (dd, 1H, J1=9.7, J2=4.5 Hz, H-4), 3.96-3.88 (m, 2H, H-3, H-6), 3.50 (dd, 1H, J1=10.0, J2=8.8 Hz, H-5), 3.75 (dd, 1H, J1=11.2, J2=2.5 Hz, H-7), 3.66 (dd, 1H, J1=11.6, J2=6.5 Hz, H-7); ‘Ring II’: δ=H 3.69-3.64 (m, 1H, H-4), 3.60 (t, 1H, J=8.9 Hz, H-5), 3.52 (ddd, 1H, J1=12.3, J2=9.7, J3=4.4 Hz, H-3), 3.42 (ddd, 1H, J1=11.9, J2=9.7, J3=4.4 Hz, H-1), 3.38-3.33 (m, 1H, H-6), 2.18 (dt, 1H, J1=12.6, J2=4.4 Hz, H-2eq), 1.52-1.17 (m, 1H, H-2ax); ‘Ring III’: δ=H 5.31 (d, 1H, J=0.5 Hz, H-1), 4.17 (dd, 1H, J1=4.8, J2=0.6 Hz, H-2), 4.10 (dd, 1H, J1=7.2, J2=4.7 Hz, H-3), 3.78-3.70 (m, 1H, H-4), 3.69-3.57 (m, 1H, H-5), 1.33 (d, 3H, J=6.7 Hz, 6-CH₃).

¹³C NMR (151 MHz, MeOD): δ=C 110.79 (C-1″), 97.41 (C-1′), 86.03 (C-4″), 85.24 (C-5), 77.47 (C-6), 76.76 (C-4), 76.47 (C-2″), 74.60 (C-6′), 73.42 (C-3), 73.31 (C-4′), 72.77 (C-3″), 72.60 (C-3′), 64.66 (C-2′), 64.13 (C-7′), 61.96 (C-1), 61.51 (C-5′), 60.86 (C-5″), 33.17 (C-2), 16.06 (C-6″, CH₃).

MALDI TOFMS: calculated for C₁₉H₃₀N₁₂O₁₁ ([M+Na]+) m/e 625.22; measured m/e 625.20.

To a stirred solution of the perazido derivative from the above reaction (230 mg, 0.381 mmol) in a mixture of THF (3 mL) and aqueous NaOH (1 mM, 5 mL), PMe₃ (1 M solution in THF, 0.22 mL, 3.0 mmol) was added. The progress of the reaction was monitored by TLC [CH₂C12/MeOH/H₂O/MeNH₂ (33% solution in EtOH), 10:15:6:15], which indicated completion after 1 hour. The reaction mixture was purified by flash chromatography on a short column of silica gel. The column was washed with the following solvents: THF (100 mL), CH₂Cl₂ (100 mL), EtOH (50 mL), and MeOH (100 mL). The product was then eluted with the mixture of 5% MeNH₂ solution (33% solution in EtOH) in 80% MeOH. Fractions containing the product were combined and evaporated under vacuum. The pure product was obtained by passing the above product through a short column of Amberlite CG50 (NH4+ form). First, the column was washed with water, then the product was eluted with a mixture of 10% NH₄OH in water to yield NB157 (123 mg, 64%) in its free base form.

For storage and biological tests, NB157 was converted to its sulfate salt form as follow: The free base form was dissolved in water, the pH was adjusted to 6.7 with H₂SO₄ (0.1 N) and lyophilized to afford the sulfate salt of NB157 as a white foamy solid.

¹H NMR (600 MHz, MeOD): ‘Ring I’: δ=H 5.25 (d, 1H, J=3.6 Hz, H-1), 4.00-3.94 (m, 1H, H-6), 3.90 (dd, 1H, J1=9.9, J2=3.5 Hz, H-5), 3.56-3.50 (m, 1H, H-3), 3.47 (dd, 1H, J1=18.3, J2=8.8 Hz, H-4), 2.66 (dd, 1H, J1=10.3, J2=3.5 Hz, H-2), 3.76 (dd, 1H, J1=11.5, J2=3.7 Hz, H-7), 3.70 (dd, 1H, J1=11.5, J2=6.4 Hz, H-7); ‘Ring II’: δ=H 3.48 (dd, 1H, J1=15.9, J2=6.7 Hz, H-5), 3.37 (dd, 1H, J1=16.5, J2=7.2 Hz, H-4), 3.18 (dd, 1H, J1=13.1, J2=5.6 Hz, H-6), 2.78 (dd, 1H, J1=9.9, J2=8.2 Hz, H-3), 2.64 (dd, 1H, J1=22.9, J2=10.3 Hz, H-1), 1.96 (dt, 1H, J1=7. 8, J2=3. 7 Hz, H-2eq), 1.23 (ddd, 1H, J1=J2=J3=12.5 Hz, H-2ax); ‘Ring III’: δ=H 5.26 (d, 1H, J=2.7 Hz, H-1), 4.05 (d, 1H, J=1.8 Hz, H-2), 4.01 (t, 1H, J=5.7 Hz, H-3), 3.56 (t, 1H, J=6.3 Hz, H-4), 3.01-2.86 (m, 1H, H-5), 1.16 (d, 3H, J=6.4 Hz, 6-CH₃).

¹³C NMR (151 MHz, MeOD): δ=C 109.78 (C-1″), 101.67 (C-1′), 88.61 (C-4″), 86.80 (C-4), 84.86 (C-5), 78.70 (C-6), 76.28 (C-2″), 75.46 (C-3′), 74.72 (C-5′), 73.79 (C-6′), 73.07 (C-4′), 72.30 (C-3″), 63.43 (C-7′), 57.55 (C-2′), 52.53 (C-3), 52.35 (C-1), 50.68 (C-5″), 49.85 (C-4), 37.64 (C-2), 19.37 (C-6″, CH₃).

MALDI TOFMS: calculated for C₁₉H₃₈N₄O₁₁ ([M+H]+) m/e 498.25; measured m/e 499.26.

Determination of Absolute Configuration at 6′-Position of NB153 and NB155:

In order to determine the absolute stereochemistry at the side-chain C6′-alcohols in NB153 and NB155, the major C6′-diasteromer alcohol 31 was synthesized, as illustrated in Scheme 3.

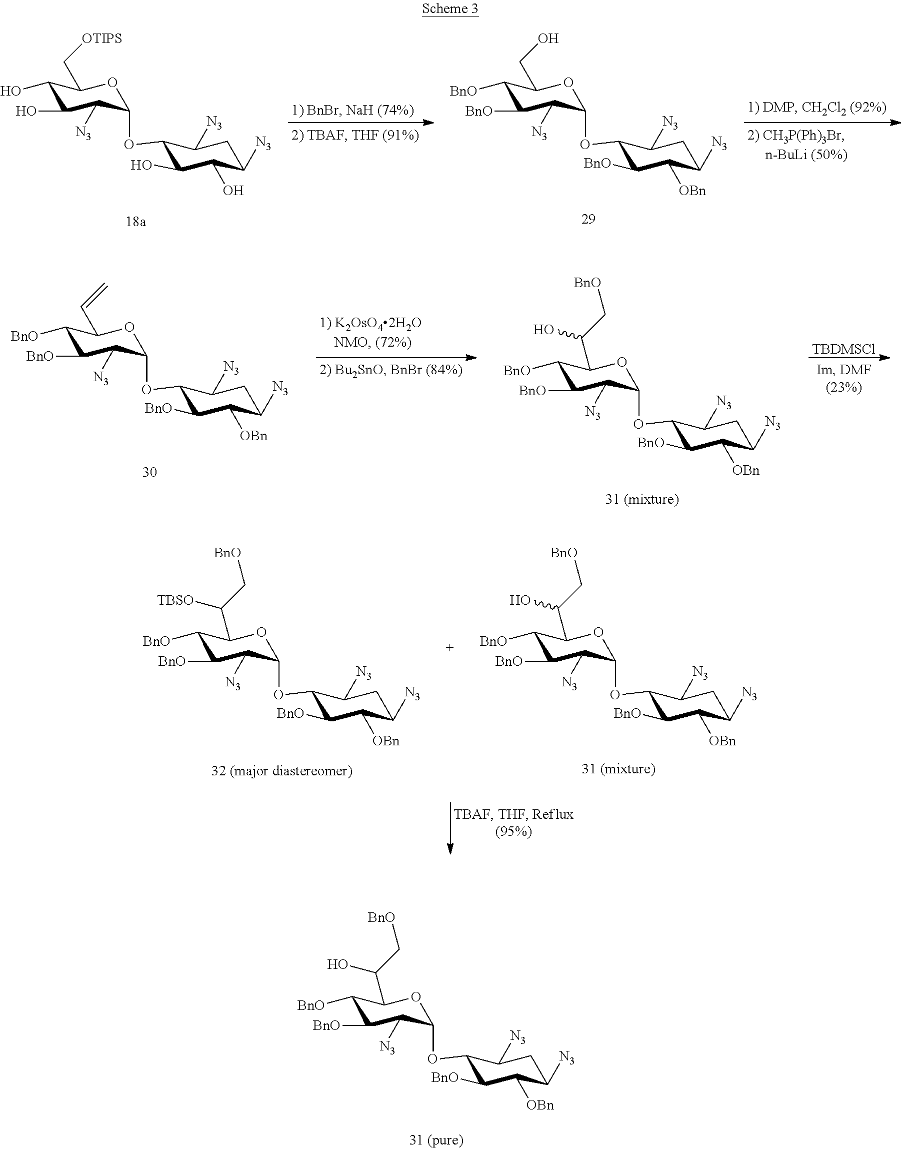

It was assumed that the change of protecting group on the secondary alcohols would improve the yields and isolation of the intermediate products at various synthetic steps experienced in the pathway in Scheme 1. The PMB protection in Scheme 1 was replaced with the benzyl protection shown in Scheme 3. Thus, the benzylation of TIPS protected Compound 18a was followed by silyl deprotection with TBAF to provide the 6′-alcohol 29 in good overall yields. Dess-Martin Periodinane (DMP) oxidation provided the corresponding aldehyde, which was treated with Wittig reagent to provide the terminal alkene 30. Dihydroxylation step was followed by selective benzylation of the primary alcohol to afford the desired 6′-alcohol 31 as a mixture of two 6′-diastereomers. Attempts to separate this mixture by using column chromatography with several different solvent systems proved unsuccessful, and it was found that the silylation of the mixture 31 with t-butyldimethylsilyl chloride (TBDMSCl) in the presence of imidazole proceeded very slow and with high selectivity of the major 6′-diastereomer. Using this advantage the silylated product of the major diastereomer 32 could be isolated in its pure form. Treatment of 32 with TBAF produced the desired product 31, which was used for configuration assignment.

To assign the absolute stereochemistry at 6′ position in compound 31, the major diastereomer 31 was separately coupled with (R)-2-methoxy-2(1-naphthyl)propanoic acid [(R)-MαNP] 33 and [(S)-MαNP] 34 of known absolute stereochemistry in presence of DCC, 4-DMAP and CSA6 to afford the respective esters (R,X)-MαNP 35 and (S,X)-MαNP 36, as shown in Scheme 4.

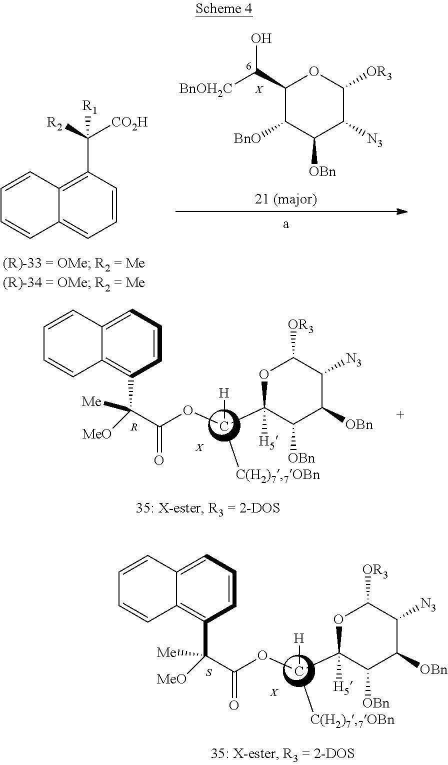

### Synthesis of ((2R,3S,4R,5R,6S)-5-azido-3, 4-bis(benzyloxy)-6-(((1R,2R,3S,4R, 6S)-4,6-diazido-2,3-bis(benzyloxy)cyclohexyl)oxy)tetrahydro-2H-pyran-2-yl)methanol (29)

To a stirred solution of the silyl ether Compound 18a (0.2 gram, 0.358 mmol) and sodium hydride (0.114 gram, 4.75 mmol) in DMF (5 mL), was added benzyl bromide (0.255 mL, 2.14 mmol) at 0° C. The reaction progress was monitored by TLC (EtOAc/Hexane 3:7). After 8 hours the reaction was completed and ice was added in small portions to quench the reaction. The mixture was diluted with ethyl acetate (30 mL) and washed with water (2×50 mL). The combined aqueous layers were extracted with diethyl ether (2×50 mL); the combined organic layers were dried over anhydrous MgSO₄, and evaporated to dryness. The residue was purified by column chromatography (EtOAc/Hexane 8:92) to yield perbenzylated silyl ether (0.243 gram, 74%).

¹H NMR (500 MHz, CDCl₃): ‘Ring I’: δ=H 5.46 (d, 1H, J=3.3 Hz, H-1), 3.97 (dd, 1H, J1=17.7, J2=8.2 Hz, H-3, H-5), 3.90 (d, 1H, J=11.6, H-6), 3.84 (d, 1H, J=11.0, H-6), 3.72-3.53 (m, 1H, H-4), 3.19 (dd, 1H, J1=10.6, J2=4.4 Hz, H-2); ‘Ring II’: δ=H 3.53 (m, 2H, H-4, H-5), 3.40 (td, 1H, J1=9.9, J2=5.3 Hz, H-1), 3.30 (ddd, 2H, J1=17.6, J2=15.1, J3=9.2 Hz, H-3, H-6), 2.21 (dd, 1H, J1=8.2, J2=4.2 Hz, H-2eq), 1.34 (dt, 1H, J1=J2=J3=12.9 Hz, H-2ax); The additional peaks in the spectrum were identified as follow: δ=H 7.28 (m, 20H, Bn), 4.94 (m, 2H, O(CH2)Bn), 4.80 (m, 6H, O(CH2)Bn), 1.14-0.95 (m, 21H, TIPS).

¹³C NMR (125 MHz, CDCl₃): δC 138.54 (Bn), 138.17 (Bn), 138.03 (Bn), 137.49 (Bn), 128.61 (Bn), 128.58 (Bn), 128.55 (Bn), 128.31 (Bn), 128.28 (Bn), 128.14 (Bn), 127.99 (Bn), 127.78 (Bn), 127.72 (Bn), 127.10 (Bn), 97.7 (C1′), 84.8, 84.62, 80.2, 77.3 76.0, 75.7, 75.2, 74.9, 72.9, 63.5 (C2′), 62.3 (C6), 60.4 (C1), 59.5, 32.5 (C2), 18.2 (TIPS), 18.1 (TIPS), 12.1 (TIPS).

To a stirred solution of perbenzylated silyl ether compound from the above step (9.24 grams, 10.0 mmol) in THF (100 mL) at 0° C., TBAF (9.0 mL, 31.0 mmol) was added and the reaction progress was monitored by TLC (EtOAc/Hexane 2:3). After 15 hours, the solvent was evaporated to dryness and the obtained residue was subjected to column chromatography (EtOAc/Hexane 3:7) to yield the corresponding perbenzylated 6′-alcohol 29 (7.0 grams, 91%).

¹H NMR (500 MHz, CDCl₃): ‘Ring I’: δ=H 5.60 (d, 1H, J=3.8 Hz, H-1), 4.11 (d, 1H, J=10.0 Hz, H-5), 4.05 (t, 1H, J=9.7 Hz, H-3), 3.83 (dd, 1H, J1=12.0, J2=2.0 Hz, H-6), 3.76 (dd, 1H, J1=12.1, J2=2.9 Hz, H-6), 3.69-3.57 (m, 1H, H-4), 3.28 (dd, 1H, J1=10.6, J2=4.6 Hz, H-2); ‘Ring II’: δ=H 3.60-3.57 (m, 2H, H-4, H-5), 3.55-3.46 (m, 1H, H-1), 3.46-3.37 (m, 2H, H-3, H-6), 2.31 (dt, 1H, J1=13.2, J2=4.5 Hz, H-2eq), 1.47 (ddd, 1H, J1=J2=J3=10.6 Hz, H-2ax); The additional peaks in the spectrum were identified as follow: δ=H 7.52-7.28 (m, 20H, Bn), 5.04 (d, 1H, J=10.8 Hz, O(CH2)Bn), 4.93 (dd, 2H, J1=10.7, J2=6.0 Hz, O(CH2)Bn), 4.90-4.86 (m, 3H, O(CH2)Bn), 4.84 (d, 1H, J=10.5 Hz, O(CH2)Bn), 4.71 (d, 1H, J=11.2 Hz, O(CH2)Bn).

¹³C NMR (125 MHz, CDCl₃): δ=C 138.0 (Bn), 138.0 (Bn), 137.8 (Bn), 137.3 (Bn), 128.6 (Bn), 128.6 (Bn), 128.5 (Bn), 128.2 (Bn), 128.1 (Bn), 128.1 (Bn), 128.0 (Bn), 128.0 (Bn), 127.7 (Bn), 127.1 (Bn), 97.7 (C1′), 84.7, 84.5, 80.1 (C3′), 77.6, 77.5, 76.0, 75.6, 75.3, 75.0, 72.0 (C5′), 63.4 (C2′), 61.4 (C6′), 60.3, 59.4, 32.4 (C2).

### Synthesis of (2R,3R,4R,5R,6R)-3-azido-4, 5-bis(benzyloxy)-2-(((1R,2R,3S,4R, 6S)-4,6-diazido-2,3-bis(benzyloxy)cyclohexyl)oxy)-6-vinyltetrahydro-2H-pyran (30)

To a solution of the 6′-alcohol 29 (1.0 gram, 1.31 mmol) in ethyl acetate (40 mL), IBX (1.1 gram, 3.92 mmol) was added in one portion. The resulting suspension was heated at 80° C. and stirred vigorously. After the reaction was completed (3.5 hours) as indicated by TLC (EtOAc/Hexane 2:3), the reaction was cooled to room temperature and filtered through Celite®. The Celite® was thoroughly washed with ethyl acetate (2×50 mL) and the combined organic layers were evaporated under reduced pressure. The crude product was subjected to flash column chromatography (EtOAc/Hexane 35:65) to yield the 6′-aldehyde (0.925 gram, 92%).

¹H NMR (500 MHz, CDCl₃): ‘Ring I’: δ=H 9.62 (s, 1H, H-6(CHO)), 5.62 (s, 1H, H-1), 4.69 (d, 1H, J=9.9 Hz, H-4), 4.01 (t, 1H, J=9.3 Hz, H-3), 3.56 (dd, 1H, J1=18.0, J2=9.1 Hz, H-5), 3.19 (d, 1H, J=14.0, H-2); ‘Ring II’: δ=H 3.56 (dd, 2H, J1=18.0, J2=9.1 Hz, H-4, H-5), 3.44 (d, 1H, J=11.7 Hz, H-1), 3.37 (t, 2H, J=8.2 Hz, H-3, H-6), 2.28 (d, 1H, J1=10.2 Hz, H-2eq), 1.44 (ddd, 1H, J1=J2=J3=14.0 Hz, H-2ax); The additional peaks in the spectrum were identified as follow: δ=H 7.27 (m, 20H, Bn), 5.00 (d, 1H, J=10.9 Hz, O(CH2)Bn), 4.92-4.75 (m, 6H, O(CH2)Bn), 4.63 (d, 1H, J=10.7 Hz, O(CH2)Bn).

¹³C NMR (125 MHz, CDCl₃): δ=C 197.2 (CHO), 138.0 (Bn), 137.5 (Bn), 137.3 (Bn), 137.1 (Bn), 128.7 (Bn), 128.6 (Bn), 128.6 (Bn), 128.6 (Bn), 128.3 (Bn), 128.3 (Bn), 128.2 (Bn), 128.1 (Bn), 97.6 (C1′), 84.6, 84.3, 80.1 (C3′), 78.4, 77.7, 76.1, 75.8, 75.3, 75.2, 62.8 (C2′), 60.3, 59.1 (C1), 32.2 (C2).

To a cooled suspension of Methyltriphenylphosphonium Iodide (0.966 gram, 2.7 mmol) in anhydrous THF at 0° C., n-BuLi (1.6 M in hexane, 0.32 mL) was added dropwise and the resulted yellow solution was stirred for an additional 30 minutes at 0° C. The 6′-aldehyde from the above step (0.822 gram, 1.08 mmol) in anhydrous THF (0.3 mL) was added at 0° C., and the reaction was allowed to stir for an additional 1.5 hour at room temperature. After completion of the reaction as indicated by TLC (EtOAc/Hexane 2:3), the reaction was quenched with saturated NH₄Cl solution. The layers were separated and the aqueous layer was extracted with ether (2×10 mL). The combined organic layers were washed with brine, dried over anhydrous MgSO₄ and evaporated to dryness. The crude product was purified by flash chromatography (EtOAc/Hexane 2.5:7.5) to yield Compound 30 (0.4 gram, 50%).

¹H NMR (400 MHz, CDCl₃): ‘Ring I’: δ=H 5.89 (ddd, 1H, J1=17.2, J2=10.4, J3=6.8 Hz, H-6), 5.56 (d, 1H, J=3.9 Hz, H-1), 5.47 (d, 1H, J=17.2 Hz, H-7trans), 5.33-5.27 (m, 1H, H-7cis), 4.64-4.56 (m, 1H, H-5), 4.09 (m, H-3), 3.32-3.27 (m, 2H, H-2, H-4); ‘Ring II’: δ=H 3.69-3.56 (m, 2H, H-4, H-5), 3.54-3.45 (m, 1H, H-1), 3.45-3.35 (m, 2H, H-3, H-6), 2.31 (dt, 1H, J1=13.2, J2=4.5 Hz, H-2eq), 1.49 (ddd, 1H, J1=J2=J3=12.6 Hz, H-2ax); The additional peaks in the spectrum were identified as follow: δ=H 7.32-7.29 (m, 20H, Bn), 5.02 (d, 1H, J=10.9 Hz, O(CH₂)Bn), 4.94 (dd, 1H, J1=9.9, J2=5.4 Hz, O(CH₂)Bn), 4.89 (d, 1H, J=6.6 Hz, O(CH₂)Bn), 4.83 (dd, 2H, J=10.7, 8.5 Hz, O(CH₂)Bn), 4.73 (d, 1H, J=10.9 Hz, O(CH₂)Bn), 4.67 (d, 1H, J=10.9 Hz, O(CH₂)Bn), 4.64-4.56 (m, 1H, O(CH₂)Bn).

¹³C NMR (100 MHz, CDCl₃): δ=C 138.2 (Bn), 138.0 (Bn), 138.0 (Bn), 137.4 (Bn), 134.9 (Bn), 128.6 (Bn), 128.5 (Bn), 128.5 (Bn), 128.5 (Bn), 128.3 (Bn), 128.2 (Bn), 128.1 (Bn), 127.9 (Bn), 127.9 (Bn), 127.7 (Bn), 127.0 (Bn), 118.9 (C7′), 97.7 (C1′), 84.7, 84.5, 82.7 (C4′), 79.7 (C3′), 77.7, 76.05, 75.6, 75.3, 75.0, 72.7 (C5′), 63.4 (C2′), 60.3 (C1), 59.3, 32.4 (C2).

### Synthesis of 1-((2R,3S,4R,5R,6S)-5-azido-3,4-bis(benzyloxy)-6-(((1R,2R,3S,4R, 6S)-4,6-diazido-2,3-bis(benzyloxy)cyclohexyl)oxy)tetrahydro-2H-pyran-2-yl)-2-(benzyloxy)ethanol (31)

To a stirred solution of Compound 30 (402 mg, 0.53 mmol) in acetone (10 mL), water (3 mL) and t-BuOH (10 mL), K2OsO₄.2H₂O (16 mg, 0.051 mmol) and NMO (0.22 mL) were added sequentially. The progress of the reaction was monitored by TLC (EtOAc/Hexane 2:3), which indicated the completion after 24 hours. The solvent was thereafter evaporated to dryness; the residue was dissolved in EtOAc to which an aqueous solution of Na₂S2O₃ was added. The layers were separated and the organic phase was washed with brine, dried over MgSO₄ and evaporated. The crude product was subjected to column chromatography (EtOAc/Hexane 1:1) to yield dihydroxylated product (300 mg, 72%) as a 6′-diasteromeric mixture.

A mixture of dihydroxylated compound (0.3 gram, 0.378 mmol) from the above step and Bu₂SnO (0.103 gram, 0.413 mmol) in toluene/MeOH (10:1, 7 mL) was refluxed for 3 hours and concentrated under reduced pressure. To a solution of this residue in toluene (3 mL) was added tetrabutylammonium bromide (0.122 gram, 0.378 mmol) and BnBr (0.09 mL, 0.756 mmol). The mixture was stirred at 85° C. overnight and quenched with addition CH₂Cl₂ (10 mL) and saturated NaHCO₃ (2 mL). After filteration through a pad of Celite®, the organic phase was washed with H₂O (3 mL), brine (5 mL), dried over MgSO₄ and concentrated under reduced pressure. The crude product was purified by column chromatography (EtOAc/Hexane 2:3) to give the Compound 31 (0.280 gram, 84%) as a 6′-diasteromeric mixture.

### Synthesis of (1-((2S,3S,4R,5R,6S)-5-azido-3,4-bis(benzyloxy)-6-(((1R,2R,3S,4R, 6S)-4,6-diazido-2,3-bis(benzyloxy)cyclohexyl)oxy)tetrahydro-2H-pyran-2-yl)-2-benzyloxy)ethoxy)(tert-butyl)dimethylsilane (32)

Compound 31 (205 mg, 0.232 mmol) was dissolved in anhydrous DMF (5 mL) and cooled to 0° C. t-butyldimethylsilyl chloride (TBSCl, 45 mg, 0.298 mmol) was added, followed by addition of Imidazole (39 mg, 0.572 mmol). The reaction mixture was allowed to attain the room temperature under stirring, and the reaction progress was monitored by TLC (EtOAc/Hexane 3:7). From TLC, reaction did not complete even after prolonged reaction times (24 hours) and at this stage the reaction was stopped by adding mixture of ethyl acetate (10 mL) and H₂O (10 mL), and the two layers were separated. The aqueous layer was thoroughly washed with ethyl acetate (4×30 mL). The combined organic layers were washed with sat. NaCl solution and dried over anhydrous MgSO₄. The solvent was evaporated to dryness and the residue was subjected to column chromatography (EtOAc/Hexane 25:75) to yield corresponding silyl ether (32) (85 mg, 23%) as a pure major diastereomer.

### Synthesis of 1-((2R,3S,4R,5R,6S)-5-azido-3,4-bis(benzyloxy)-6-(((1R,2R,3S,4R,6S)-4, 6-diazido-2,3-bis(benzyloxy)cyclohexyl)oxy)tetrahydro-2H-pyran-2-yl)-2-(benzyloxy)ethanol (31 as pure major diastereomer)

To a stirred solution of Compound 32 (60 mg, 0.06 mmol) in THF (3 mL) at room temperature, TBAF (0.052 mL, 0.179 mmol) was added and the reaction was refluxed at 50° C. overnight. After completion of the reaction as indicated by TLC (EtOAc/Hexane 2:3), the solvent was evaporated to dryness and the obtained residue was subjected to column chromatography (EtOAc/Hexane 3:7) to yield single diastereromer 31 (52 mg, 95%).

¹H NMR (500 MHz, CDCl₃): ‘Ring I’: δ=H 5.53 (d, 1H, J=3.9 Hz, H-1), 4.17 (dd, 1H, J1=10.0, J2=2.4 Hz, H-5), 4.12 (m, 1H, H-6), 3.96 (dd, 1H, J1=10.3, J2=8.9 Hz, H-3), 3.69-3.61 (m, 1H, H-4), 3.50-3.45 (m, 2H, H-7, H-7), 3.22 (dd, 1H, J1=10.3, J2=3.9 Hz, H-2), 3.59 (BrS, 1H, 6′-OH); ‘Ring II’: δ=H 3.58-3.49 (m, 2H, H-4, H-5), 3.44-3.11 (m, 3H, H-1, H-3, H-6), 2.23 (dt, 1H, J1=13.2, J2=4.5 Hz, H-2eq), 1.38 (ddd, 1H, J1=J2=J3=12.6 Hz, H-2ax); The additional peaks in the spectrum were identified as follow: δ=H 7.29-7.23 (m, 25H, Bn), 4.98 (d, 1H, J=10.8 Hz, O(CH₂)Bn), 4.92-4.74 (m, 6H, O(CH₂)Bn), 4.65 (d, 1H, J=11.1 Hz, O(CH₂)Bn), 4.42 (q, 2H, J=11.9 Hz, O(CH₂)Bn).

¹³C NMR (125 MHz, CDCl₃): δ=C 138.0 (Bn), 138.0 (Bn), 137.9 (Bn), 137.7 (Bn), 137.3 (Bn), 128.6 (Bn), 128.6 (Bn), 128.5 (Bn), 128.5 (Bn), 128.5 (Bn), 128.3 (Bn), 128.1 (Bn), 128.1 (Bn), 128.0 (Bn), 127.9 (Bn), 127.8 (Bn), 127.7 (Bn), 127.6 (Bn), 127.0 (Bn), 97.4 (C1′), 84.6, 84.4, 80.8, 78.4 (C4′), 77.5, 76.0 (Bn), 75.6 (Bn), 75.3 (Bn), 74.6 (Bn), 73.4 (Bn), 71.8, 71.6, 71.2 (C7′), 63.3 (C2′), 60.2 (C1), 59.5 (C3), 32.4 (C2).

### Synthesis of (R,X)-Ester

A mixture of (R)-2-methoxy-2(1-naphthyl)propanoic acid [(R)-MαNP] (0.01 gram, 0.04 mmol), 4-dimethylaminopyridine (DMAP, 0.006 gram, 0.049 mmol), 10-camphorsulfonic acid (CSA, 0.002 gram, 0.008 mmol), and 1,3-dicyclohexylcarbodiimide (DCC, 0.047 gram, 0.22 mmol) was stirred in CH₂Cl₂ (3 mL) at 0° C. The major alcohol 31 from the above (0.038 gram, 0.043 mmol) was dissolved in CH₂Cl₂ (2 ml), slowly added to the above stirred mixture, and the reaction was left at room temperature for 72 hours. The mixture was diluted with EtOAc and washed with 1% HCl solution, saturated NaHCO₃ and brine. The combined organic layer was dried over MgSO₄, evaporated and subjected to a column chromatography (EtOAc/Hexane) to yield the desired ester (R,X)-35 (0.008 gram, 17%).

¹H NMR (600 MHz, CDCl₃): ‘Ring I’: δ=H 5.55 (dd, 1H, J=9.9, 3.7 Hz, H-6), 4.87 (d, 1H, J=3.4 Hz, H-1), 3.86 (d, 1H, J=10.0 Hz, H-4), 3.50-3.46 (m, 1H, H-7), 3.38 (d, 1H, J=10.2 Hz, H-3), 3.36-3.32 (m, 1H, H-7), 1.55-1.50 (m, 1H, H-5), 1.28 (dd, 1H, J1=10.4, J2=3.9 Hz, H-2), ‘Ring II’: δ=H 3.51 (d, 1H, J1=9.7 Hz, H-6), 3.43 (dt, 3H, J1=12.1, J2=7.8 Hz, H-4, H-5, H-3), 3.25 (ddd, 1H, J1=12.6, J2=10.0, J3=4.6 Hz, H-1), 2.23 (dd, 1H, J1=10.9, J2=6.5 Hz, H-2eq), 1.46-1.39 (m, 1H, H-2ax); The additional peaks in the spectrum were identified as follow: 6=8.39 (d, 1H, J=8.7 Hz, Ar), 7.80-7.75 (m, 2H, Ar), 7.63 (d, 1H, J=6.4 Hz, Ar), 7.54 (t, 2H, J=7.6 Hz, Ar), 7.47-7.40 (m, 2H, Ar), 7.38 (d, 2H, J=7.1 Hz, Ar), 7.37-7.33 (m, 2H, Ar), 7.32-7.27 (m, 9H, Ar), 7.23 (ddd, 4H, J1=6.5, J2=4.7, J3=2.2 Hz, Ar), 7.20 (d, 3H, J=8.0 Hz, Ar), 7.09 (ddd, 1H, J1=8.5, J2=6.8, J3=1.5 Hz, Ar), 7.06-7.02 (m, 1H, Ar), 6.96-6.92 (m, 2H, Ar), 5.01 (d, 1H, J=11.2 Hz, O(CH₂)Bn), 4.88 (d, 2H, J=4.1 Hz, O(CH₂)Bn), 4.84 (d, 1H, J=10.8 Hz, O(CH₂)Bn), 4.59 (d, 1H, J=11.4 Hz, O(CH₂)Bn), 4.50 (d, 1H, J=11.3 Hz, O(CH₂)Bn), 4.44 (d, 1H, J=11.8 Hz, O(CH₂)Bn), 4.25 (d, 1H, J=11.9 Hz, O(CH₂)Bn), 3.99 (d, 1H, J=11.3 Hz, O(CH₂)Bn), 3.71 (d, 1H, J=11.3 Hz, O(CH₂)Bn), 3.07 (s, 1H, OCH₃), 2.02 (s, 3H, CH₃).

¹³C NMR (125 MHz, CDCl₃): δ=C 173.3 (Ar), 138.5 (Ar), 138.4 (Ar), 137.9 (Ar), 137.7 (Ar), 137.3 (Ar) 135.3 (Ar), 134.2 (Ar), 131.8 (Ar), 130.1 (Ar), 128.9 (Ar), 128.65 (Ar), 128.62 (Ar), 128.5 (Ar), 128.46 (Ar), 128.44 (Ar), 128.22 (Ar), 128.22 (Ar), 127.76 (Ar), 127.71 (Ar), 127.5 (Ar), 127.4 (Ar), 127.2 (Ar), 126.7 (Ar), 126.4 (Ar), 126.3 (Ar), 126.2 (Ar), 124.8 (Ar), 99.7 (C1′), 84.5, 84.43 (s), 81.1, 79.8, 77.0, 76.7, 76.1, 75.1, 74.2, 74.2, 73.7, 72.7, 70.2, 69.8, 61.8, 60.2, 59.1, 50.7, 32.3, 31.1, 29.8, 21.5 (CH₃).

### Synthesis of (S,X)-Ester (36)

A mixture of (S)-2-methoxy-2(1-naphthyl)propanoic acid [(S)-MαNP] (0.007 gram, 0.03 mmol), 4-dimethylaminopyridine (DMAP, 0.005 gram, 0.04 mmol), 10-camphorsulfonic acid (CSA, 0.001 gram, 0.004 mmol), and 1,3-dicyclohexylcarbodiimide (DCC, 0.034 gram, 0.16 mmol) was stirred in CH₂Cl₂ (3 mL) at 0° C. The major alcohol 31 from the above (0.028 gram, 0.031 mmol), was dissolved in CH₂Cl₂ (2 ml), slowly added to the above stirred mixture, and the reaction was left at room temperature for 72 hours. The mixture was diluted with EtOAc and washed with 1% HCl solution, saturated NaHCO₃ and brine. The combined organic layer was dried over MgSO₄, evaporated and subjected to a column chromatography (EtOAc/Hexane) to yield the desired ester (S,X)-36 (0.007 gram, 20%).

¹H NMR (600 MHz, CDCl₃): ‘Ring I’: δ=H 5.49 (dd, 1H, J=8.5, 4.4 Hz, H-6), 5.17 (d, 1H, J=3.8 Hz, H-1), 4.04 (d, 1H, J=10.0 Hz, H-4), 3.58 (t, 1H, J=9.8 Hz, H-3), 3.25 (d, 1H, J=8.5 Hz, H-7), 3.22 (dd, 1H, J1=10.7, J2=4.6 Hz, H-7), 2.34 (dd, 1H, J1=17.0, J2=6.3 Hz, H-5), 2.12-2.02 (m, 1H, H-2) ‘Ring II’: δ=H 3.51 (dt, 2H, J1=17.8, J2=9.3 Hz, H-4, H-5), 3.46-3.37 (m, 2H, H-1, H-6), 3.33-3.27 (m, 1H, H-3), 2.25 (dt, 1H, J1=13.2, J2=4.5 Hz, H-2eq), 1.43 (ddd, 1H, J1=J2=J3=12.6 Hz, H-2ax); The additional peaks in the spectrum were identified as follow: δ=H 8.08 (d, 1H, J=8.8 Hz, Ar), 7.89 (d, 1H, J=7.3 Hz,), 7.76 (dd, 2H, J1=15.9, J2=8.1 Hz, Ar), 7.46 (t, 2H, J=7.5 Hz,), 7.44-7.41 (m, 1H, Ar), 7.39 (d, 1H, J=7.5 Hz, Ar), 7.36 (t, 2H, J=7.3 Hz, Ar), 7.34-7.27 (m, 8H, Ar), 7.25-7.23 (m, 2H, Ar), 7.23-7.19 (m, 6H, Ar), 7.16 (t, 1H, J=7.1 Hz, Ar), 7.14-7.09 (m, 3H, Ar), 6.91-6.87 (m, 2H, Ar), 5.01 (d, 1H, J=11.1 Hz, O(CH₂)Bn), 4.90-4.79 (m, 3H, O(CH₂)Bn), 4.63 (q, 2H, J=11.1 Hz, O(CH₂)Bn), 4.22-4.15 (m, 2H, O(CH₂)Bn), 4.12 (d, 1H, J=11.0 Hz, O(CH₂)Bn), 3.66 (d, 1H, J=11.0 Hz, O(CH₂)Bn), 3.29 (S, 3H, OCH₃), 1.97 (s, 3H, CH₃).

¹³C NMR (125 MHz, CDCl₃): δ=C 172.7 (Ar), 138.3 (Ar), 138.0 (Ar), 137.9 (Ar), 137.7 (Ar), 137.3 (Ar) 134.1 (Ar), 130.5 (Ar), 129.3 (Ar), 129.1 (Ar), 128.6 (Ar), 128.6 (Ar), 128.4 (Ar), 128.4 (Ar), 128.3 (Ar), 128.3 (Ar), 128.2 (Ar), 127.9 (Ar), 127.7 (Ar), 127.7 (Ar), 127.6 (Ar), 127.6 (Ar), 127.5 (Ar), 126.9 (Ar), 126.1 (Ar), 125.6 (Ar), 125.1 (Ar), 125.1 (Ar), 124.6 (Ar), 97.1 (C1′), 84.5 (C5), 84.5 (C4), 81.2, 80.1 (C3′), 77.9 (C5′), 77.3, 77.0 (C4), 76.1, 75.2, 74.8, 74.4, 74.3 (C6′), 72.8, 70.4 (C4′), 69.4 (C7′), 62.5 (C2′), 60.2 (C3), 59.1 (C1), 51.4 (OCH₃), 32.3 (C2), 29.85, 21.9 (CH₃).

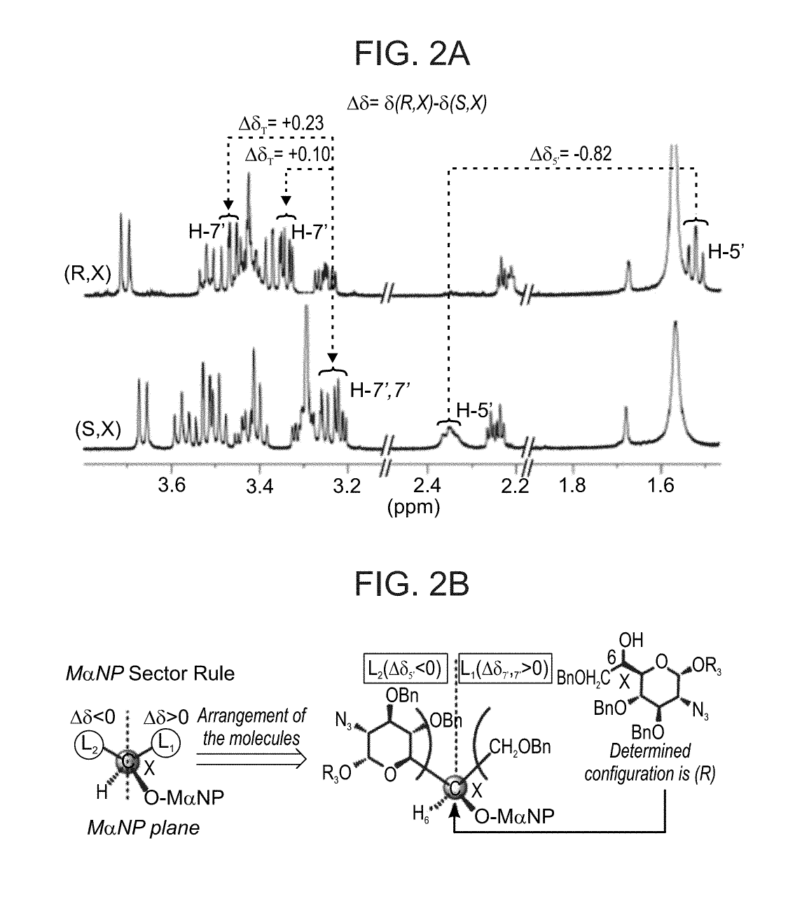

The absolute stereochemistry at the 6′ position (denoted by X) was then determined by ¹H NMR magnetic anisotropy, which is based on Sector rule 7 and relays on the difference in chemical shift values for the assigned protons in the NMR spectra (see, FIGS. 2A-B). As shown in FIG. 2A, the difference in chemical shift [Δδ=δ(R, X)−δ(S, X)] for H-5′(−0.82) was negative, while that for H-7′, 7′ (+0.23, +0.10) was positive. According to the Sector rule shown in FIG. 2B, the structures (R, X)-MαNP 35 and (S, X)-MαNP 36 are arranged such that OMαNP is positioned on the front and H-6′ on the back, while the Δδ positive and Δδ negative parts are positioned on the right and left sides, respectively. These data confirms the R configuration (X═R) at the 6′ carbon atom in compound 31.

This study establishes that the major and minor diastereomers, compounds NB153 and NB155, exhibit (R)- and (S)-configuration at 6′ position: 6′-(R)-NB153 and 6′-(S)-NB155.

### Example 2

### Activity Assays of Exemplary Compounds of Example 1

The experimental assay procedure and result analysis was carried out essentially as described hereinabove and in further detail hereinunder.

Materials and Methods:

In all biological tests, all the tested aminoglycosides were in their sulfate salt forms [Mw (gr/mol) of the sulfate salts were as follow: Compound 1-437.1, NB74—564.3, NB124—605.9, NB153—526.8, NB155—512.2, NB156—705.9, NB157—746.6, G418—692.7, gentamicin—653.2].

Dual Luciferase Readthrough Assays:

DNA fragments derived from PCDH15, CFTR, and IDUA cDNAs, including the tested nonsense mutation or the corresponding wild type (wt) codon, and four to six upstream and downstream flanking codons were created by annealing the following pairs of complementary oligonucleotides:

Usher Syndrome:
p.R3Xmut/wt:
5′-GATCCCAGAAGATGTTTT/CGACAGTTTTATCTCTGGACAGAGC
T-3′
and
5′-CTGTCAGAGATAAAACTGTCA/GAAACATCTTCTG-3′;
p.R245Xmut/wt:
5′GATCCAAAATCTGAATGAGAGGT/CGAACCACCACCACCACCCTCGA
GCT-3′
and
5′-CGAGGGTGGTGGTGGTTGTTCG/ACCTCTCATTCAGATTTTG-3′;
Cystic Fibrosis:
p.G542Xmut/wt:
5′-TCGACCAATATAGTTCTTT/GGAGAAGGTGGAATCGAGCT-3′
and
and
5′-CGATTCCACCTTCTCA/GAAGAACTATATTGG-3′;
Hurler Syndrome:
p.Q70Xmut/wt:
5′-TCGACCCTCAGCTGGGACT/CAGCAGCTCAACCTCGAGCT-3′
and
5′-CGAGGTTGAGCTGCTA/GGTCCCAGCTGAGG-3′.

Fragments were inserted in frame into the polylinker of the p2Luc plasmid between either BamHI and SacI (p.R3X and p.R245X) or SalI and SacI (all the rest) restriction sites. For the in vitro readthrough assays, the obtained plasmids, with addition of the tested aminoglycosides were transcribed and translated using the TNT Reticulocyte Lysate Quick Coupled Transcription/Translation System. Luciferase activity was determined after 90 minutes of incubation at 30° C., using the Dual Luciferase Reporter Assay System (Promega™). Stop codon readthrough was calculated as previously described [Grentzmann et al. RNA 1998, 4, 479-486].

Protein Translation Inhibition Tests:

Prokaryotic in vitro translation inhibition by the different aminoglycosides was quantified in coupled transcription/translation assays by using E. coli S30 extract for circular DNA with the pBESTluc plasmid (Promega), according to the manufacturer's protocol. Translation reactions (25 μL) that contained variable concentrations of the tested aminoglycoside were incubated at 37° C. for 60 minutes, cooled on ice for 5 minutes, and diluted with a dilution reagent (tris-phosphate buffer (25 mM, pH 7.8), DTT (2 mM), 1,2-diaminocyclohexanetetraacetate (2 mM), glycerol (10%), Triton X-100 (1%) and BSA (1 mg mL-1)) into 96-well plates. Eukaryotic in vitro translation inhibition was quantified by use of TNT® T7 Quick Coupled Transcription/Translation System with a luciferase T7 control DNA plasmid (Promega), according to the manufacturer protocol. Translation reactions (25 μL) containing variable concentrations of the tested aminoglycoside were incubated at 30° C. for 60 minutes, cooled on ice for 5 minutes, diluted with the dilution reagent and transferred into 96-well plates. In both prokaryotic and eukaryotic systems the luminescence was measured immediately after the addition of the Luciferase Assay Reagent (50 μL; Promega), and the light emission was recorded with a FLx800 Fluorescence Microplate Reader (Biotek). The half-maximal inhibition concentration (IC50) values were obtained from fitting concentration-response curves to the data of at least two independent experiments by using Grafit 5 software.

Antibacterial Activity Tests:

Comparative antibacterial activities were determined in two representative strains of Gram-negative (E. coli R477-100) and Gram-positive (B. subtilis ATCC-6633) bacteria, by measuring the MIC values using the double-microdilution method according to the National Committee for Clinical Laboratory Standards (NCCLS) (NCCLS. National Committee for Clinical Laboratory Standards, Performance standards for antimicrobial susceptibility testing. Fifth information supplement: Approved Standard M100-S5; Villanova, Pa.: NCCLS, 1994.). All the experiments were performed in triplicates and analogous results were obtained in three different experiments.

Results:

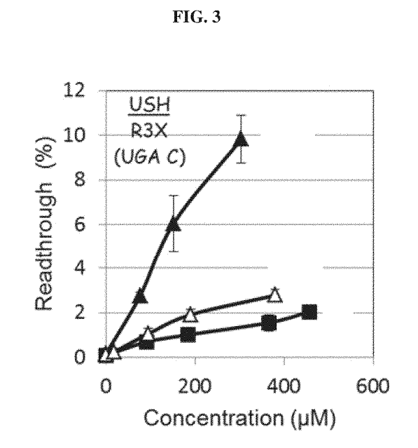

FIG. 3 presents comparative plots showing in vitro stop codon suppression levels induced by Compound 1 (-▪-), NB153 (-▴-), and NB155 (-Δ-) in R3X nonsense mutation construct representing USH1 genetic disease.

These comparative PTC suppression activity tests show that installation of C7′-hydroxyl group (NB153) on Compound 1 dramatically increases its in vitro readthrough activity, and is more pronounced than the effect of NB155. These data show an improved activity attributed to the additional hydroxyl group, and further emphasize the role of stereochemistry at 6′-position in RNA target recognition. The observed somewhat higher activity of NB155 to that of Compound 1 suggests that the additional 7′-hydroxyl in NB155 can overcome the configurational penalty at 6′ position.

The impact of the additional 7′-hydroxyl in Compounds NB156 and NB157 was evaluated against previously published compounds NB74 and NB124, which differ from NB156 and NB157 by the absence of the 7′-hydroxyl, as shown below.

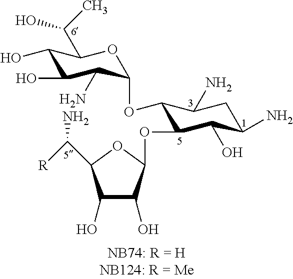

Activity was tested using a collection of dual-luciferase reporter plasmids, containing different sequence contests around premature stop codons derived from the PCDH15, CFTR, and IDUA genes that underline USH1, CF, and MPS I-H, respectively. The exemplified nonsense reporters were R3X and R245X for USH1, G542X for CF, and Q70X for MPS I-H.

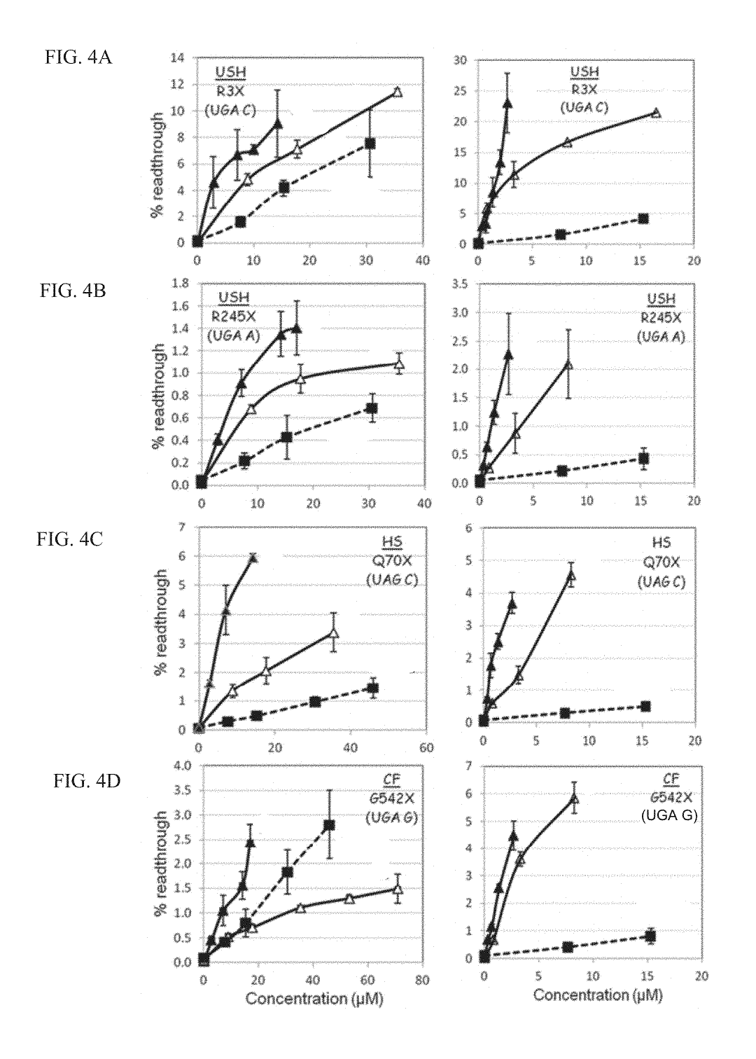

The obtained data is presented in FIGS. 4A-D, showing comparative plots showing in vitro stop codon suppression levels induced by NB74 (-Δ-), NB156 (-▴-), and gentamicin (--▪--) (left) and by NB124 (-Δ-), NB157 (-▴-), and gentamicin (--▪--) (right), in nonsense constructs representing R3X (USH1) (FIG. 4A), R245X (USH1) (FIG. 4B), Q70X (HS) (FIG. 4C), and G542X (CF) (FIG. 4D). The results are averages of at least three independent experiments.

As clearly shown in FIGS. 4A-D, the positive impact of the C7′-hydroxyl group shown for NB153 is retained also in the pseudo-trisaccharides. In all mutations tested, the readthrough activity of NB156 is substantially better than that of the structurally related NB74, and the activity of NB157 is better than its structurally related NB124. In addition, in all mutations tested, the activities of both NB156 and NB157 were significantly better than that of the clinical drug gentamicin.

In order to evaluate the specificity toward eukaryotic cytoplasmic ribosome, comparative protein translation inhibition of Compounds NB74, NB124, NB156 and NB157 in eukaryotic system was determined, using coupled transcription/translation assays.

In all biological tests, all AGs were in their sulfate salt forms, and the concentrations refer to the free amine form of each AG. The eukaryotic and prokaryotic half-maximal-inhibition values (IC₅₀^{Euk} and IC₅₀^{Pro}) were quantified in coupled transcription/translation assays by using active luciferase detection as previously described. Minimal inhibitory concentration (MIC) values were determined by using the double-microdilution method.

The obtained data in presented in Table 2 below.

TABLE 2

| | | | Antibacterial | |
|---|---|---|---|---|
| | | | Activity MIC (μM) | |
| | Translation Inhibition | | E. Coli R | B. Subtilis |
| Compound | IC₅₀^{Euk} (μM) | IC₅₀^{Pro} (μM) | 477/100 | ATCC6633 |
| Gentamicin | 62 ± 9 | 0.03 ± 0.00 | 6 | <0.75 |
| G418 | 2.0 ± 3 | 0.01 ± 0.00 | 9 | <1.25 |
| Compound 1 | 347.1 ± 34.3 | 6.0 ± 1.0 | | |
| NB153 | 120.5 ± 14.5 | 11.0 ± 1.2 | >311 | 311 |
| NB155 | 515.8 ± 15 | 91.9 ± 8.4 | >375 | >375 |
| NB74 | 13.9 ± 1.2 | 1.0 ± 0.1 | 680 | 42 |
| NB156 | 7.5 ± 0.5 | 0.7 ± 0.1 | >273 | 34 |
| NB124 | 1.5 ± 0.1 | 1.1 ± 0.2 | 1267 | 156 |
| NB157 | 1.2 ± 0.1 | 1.2 ± 0.1 | >257 | 64 |

The obtained data indicates that the efficacy with which NB157 (half-maximal inhibitory concentration value IC₅₀^{Euk}=1.2 μM) inhibits eukaryotic translation is greater than that of NB156 (IC₅₀^{Euk}=13.9 μM) and gentamicin (IC₅₀^{Euk}=62 μM), similarly to the PTC suppression activity shown in FIGS. 4A-D. In addition, NB156 and NB157 are 1.85-fold and 1.25-fold more specific to the eukaryotic ribosome than their structurally related Compounds NB74 and NB124, respectively. These data indicate that the elevated PTC suppression activities of NB156 and NB157 are associated with their increased specificity to the eukaryotic ribosome.

The measured IC₅₀^{Pro} and MIC values in Table 2 show that the efficacy with which NB156 and NB157 inhibit the prokaryotic ribosome and their subsequent antibacterial activity are very similar to those of their structurally related Compounds NB74 and NB124, respectively. The observed similar impact on bacterial ribosome by these compounds suggest that NB156 and NB157 are less ototoxic than gentamicin and G418.

Thus, a new pharmacophoric point, 7′-hydroxyl group, is shown herein as a valuable structural element of the glucosamine ring (Ring I) that significantly affects eukaryotic versus prokaryotic selectivity and the subsequent PTC suppression activity.

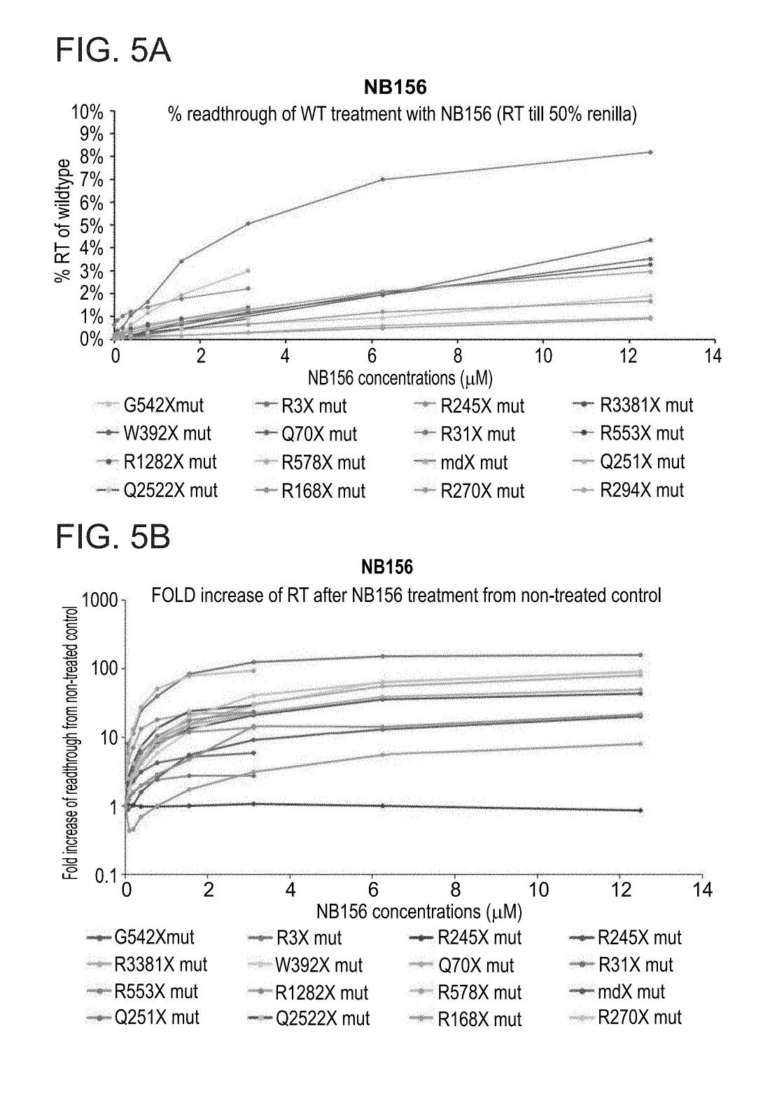

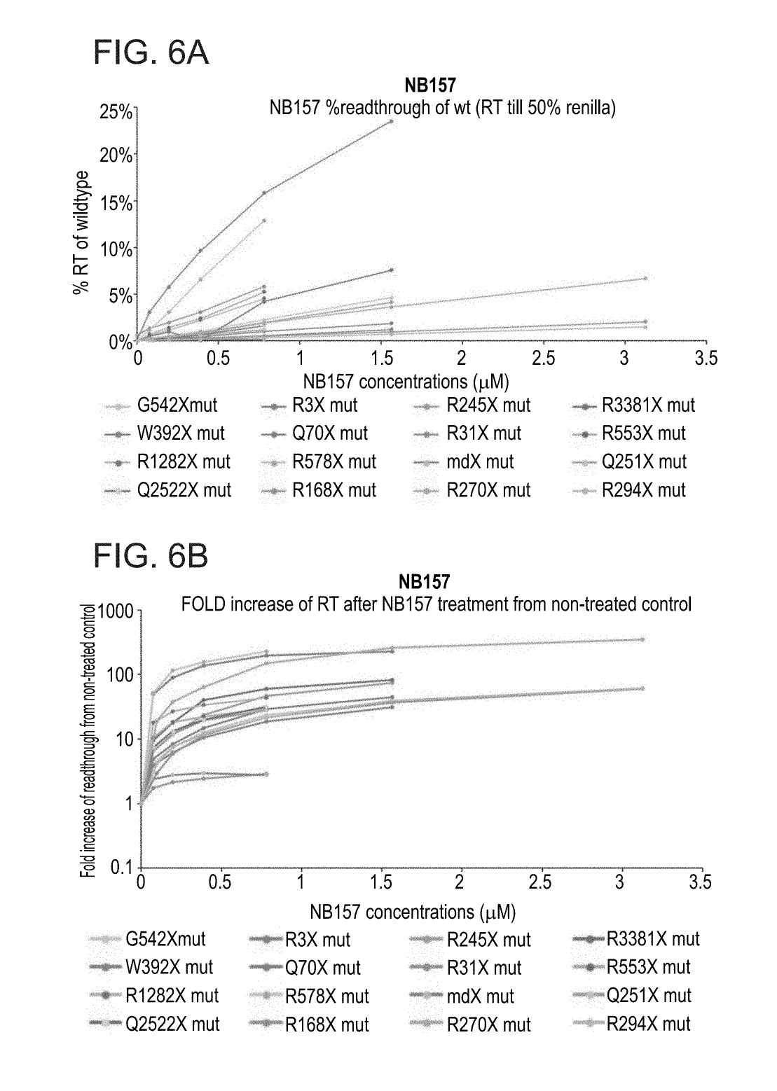

Further assays were conducted essentially as described hereinabove, and some of the obtained data is presented in FIGS. 5A-6B.

In these assays, the readthrough of a broad arsenal of stop codon mutations in the presence of NB156 and NB157 was tested. Briefly, NB156 and NB157 were tested at escalating doses for their read-through properties towards the nonsense mutation using the wild-type (WT) sequence of each specific complementary DNA (cDNA) as a control, and plasmids bearing the stop codon mutations in a dual-luciferase assay. DNA fragments derived from different cDNAs were prepared using either the WT or nonsense mutation, in which the sequences from the mutant or wild-type codon were surrounded by four to six upstream and downstream flanking codons. The cDNA sequence was inserted into the polylinker of the p2luc plasmid for each sequence.

The tested mutations and the genetic diseases associated therewith are shown in Table 3 below.

TABLE 3

| Mutation | Disease |
|---|---|
| G542X | Cystic fibrosis |
| R553X | Cystic fibrosis |
| W1282X | Cystic fibrosis |
| R3381X | Duchenne muscular dystrophy |
| Q2522X | Duchenne muscular dystrophy |
| mdx | Duchenne muscular dystrophy |
| | (mouse) |
| W392X | Hurler Syndrome |
| Q70X | Hurler Syndrome |
| R168X | Rett Syndrome |
| R270X | Rett Syndrome |
| R294X | Rett Syndrome |
| R578X | Severe epidermolysis bullosa |
| Q251X | Severe epidermolysis bullosa |
| R3X | Usher Syndrome |
| R245X | Usher Syndrome |
| R31X | Usher Syndrome |

FIG. 5A presents comparative stop-codon mutation readthrough plots, showing percent readthrough as a function of concentration of WT with NB156 (readthrough to 50% renilla), comparing the readthrough of the mutations G542X, W392X, R1282X, Q2522X, R3X, Q70X, R578X, R168X, R245X, R31X, mdX, R270X, R3381X, R553X, Q251X and R294X.

FIG. 5B presents comparative stop-codon mutation readthrough plots, showing fold increase of readthrough after exposure to NB156 from non-treated control as a function of NB156 concentration, comparing the readthrough of the mutations G542X, W392X, R1282X, Q2522X, R3X, Q70X, R578X, R168X, R245X, R31X, mdx, R270X, R3381X, R553X, Q251X and R294X.

FIG. 6A presents comparative stop-codon mutation readthrough plots, showing percent readthrough as a function of concentration of WT with NB157 (readthrough to 50% renilla), comparing the readthrough of the mutations G542X, W392X, R1282X, Q2522X, R3X, Q70X, R578X, R168X, R245X, R31X, mdX, R270X, R3381X, R553X, Q251X and R294X (see, Table 3 above).

FIG. 6B presents comparative stop-codon mutation readthrough plots, showing fold increase of readthrough after exposure to NB157 from non-treated control as a function of NB157 concentration, comparing the readthrough of the mutations G542X, W392X, R1282X, Q2522X, R3X, Q70X, R578X, R168X, R245X, R31X, mdX, R270X, R3381X, R553X, Q251X and R294X (see, Table 3 above).

In additional comparative assays, the stop-codon mutation readthrough activity of NB156 was compared to that of NB74. In all tested mutations, NB156 was shown to be more active than NB74.

These data further demonstrate the readthrough activity exhibited by NB156 and NB157 on various stop codon mutations.

### Example 3

### Unsaturated Glucosamine (Ring I)-Containing Exemplary Compounds According to Some Embodiments of the Present Invention

Exemplary new modifications of aminoglycoside structures were performed by inserting unsaturation at ring I (glucosamine ring). It has been assumed that by the deletion of C4′-OH or C3′,C4′-hydroxyls with a simultaneous introduction of unsaturation on Ring I makes the ring relatively “free” to move within the binding pocket for better pseudo-pair interaction with G1408 and improved 7t-7t stacking with A1491.

### Chemical Syntheses

The following exemplary aminosugars Compounds NB154, NB158 and NB159 were synthesized:

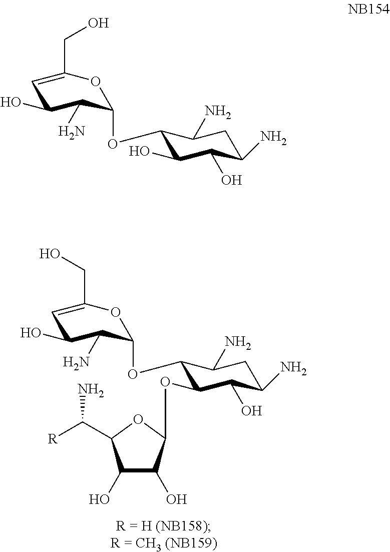

All the structures were confirmed and characterized by a combination of various 1D and 2D NMR techniques, including 1D TOCSY, 2D COSY, 2D ¹H-¹³C HMQC and HMBC along with mass spectrometry.

### Synthesis of NB154

The synthesis of NB154 is depicted in Scheme 5 below.

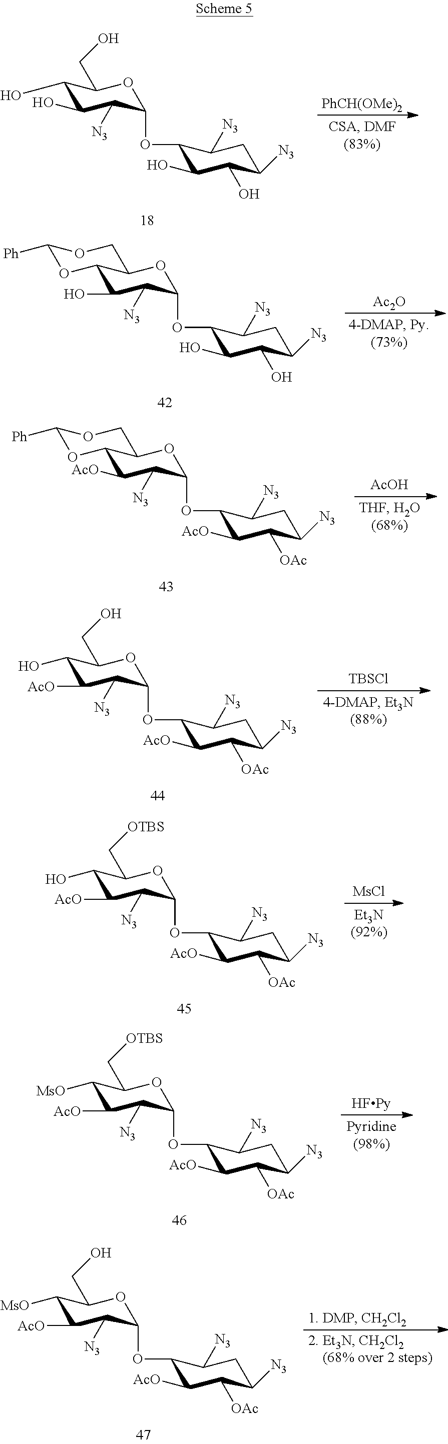

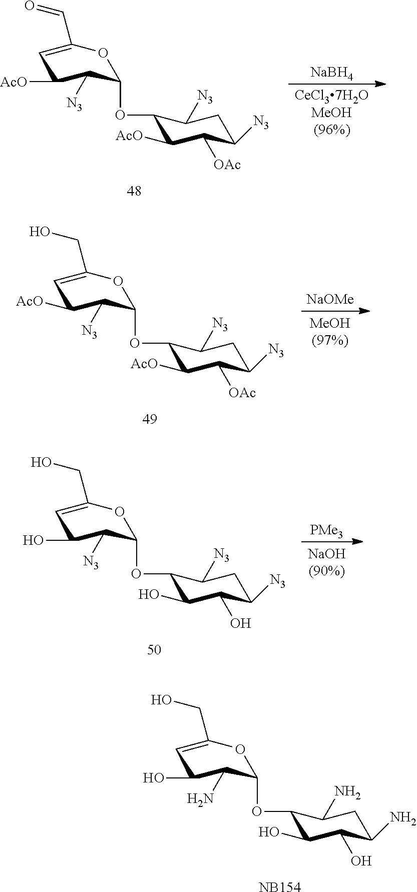

Briefly, the synthesis started from paromamine, which is obtained from commercially available paromomicin sulfate under acidic (HCl/MeOH) hydrolysis, as previously described. Initially, paromamnie was converted into the triazide by the action insitu generated triflic azide from triflic anhydride and NaN₃ in the presence of CuSO₄ to yield paromamine perazide (18), as described in further detail hereinabove. Upon obtaining the paromamine perazide, the 4′,6′-OH groups were converted into corresponding bebzylidene acetal (42) using benzaldehyde dimethyl acetal under acidic conditions. The other hydroxy groups were converted to acetate esters in presence of acetic anhydride under basic conditions (43). Deprotection of the arylidene group in Compound 43 under mild acidic environment led to the formation of diol 44 which was subjected to further post functional transformations to yield the desired compound. In order to differentiate the 4′-OH and 6′-OH groups so as to perform selective oxidation, the 6′-OH group in compound 43 was protected as its silyl ether 45 by selective protection with tert-butyldiphenylsilyl chloride (TBDPSCl), while the other hydroxyl group was masked as mesylate ester using mesyl chloride (MsCl) under base condition (Et₃N) to thereby obtain Compound 46 in excellent yields.

In order to avoid the hydrolysis of 4′-OMs ester functionality during the silyl deprotection using TBAF, the TBAF reaction mixture was buffered with AcOH and obtained the 6′-OH functional molecule 47 leaving 4′-OMs ester intact with the molecule. DMP oxidation of the C-6′ hydroxyl group, followed by concomitant elimination of 4′-OMs ester under basic conditions in one-pot reaction lead to the formation of corresponding α,β-unsaturated aldehyde 48 in good yield. Compound 48, upon Luche reduction conditions gave allylic alcohol 49, which on treatment with NaOMe followed by Staudinger reaction yielded the pseudo-disaccharide NB154,

### Synthesis of 1,3,2′-perazido-paromamine (18)

Paromomicin sulfate was hydrolyzed under acidic conditions (HCl/MeOH) to paromamine. Paromamine was converted into the triazide by the in situ generated triflic azide from triflic anhydride and NaN₃ in the presence of CuSO₄.

### Generation of Triflic Azide

To a vigorously stirred solution of NaN₃ (3.6 grams, 18 equiv.) in water (9.0 mL) and Toluene (9.0 mL) at 0° C., triflic anhydride (4.6 mL, 9.0 equiv.) was added drop wise and the reaction mixture was stirred for 30 minutes at 0° C. The temperature was thereafter raised to 10° C. and the biphasic system was stirred for 2 hours. Saturated aqueous NaHCO₃ was then added dropwise until the gas evaluation ceased. The phases were separated and the aqueous phase was extracted with toluene (2×9 mL). The combined organic layers were used in the diazo transfer reaction.

### Diazo Transfer Reaction

Paromamine (1.0 gram, 1.0 equiv.), NaHCO₃ (3.1 grams, 12.0 equiv.) and copper (II) sulfate were dissolved in water (5.0 mL). Triflic azide stock solution was added, followed by the addition of methanol (40 mL), to thereby obtain a homogeneous solution. The blue color reaction mixture was stirred vigorously at room temperature. Complete conversion of amine was indicated by the change of blue color to green. After stirring for 48 hours, TLC (EtOAc/MeOH 95:5) analysis indicated the completion of the reaction. The solvent was hereafter evaporated to dryness and the residue was subjected to column chromatography (EtOAc 100%).

¹H NMR (500 MHz, MeOD): ‘Ring I’: δ_{H}=5.69 (d, 1H, J=3.7 Hz, H-1), 3.99 (ddd, 1H, J=9.9, 4.1, 2.6 Hz, H-5), 3.94 (dd, 1H, J=10.2, 9.1 Hz, H-3), 3.84 (dd, 1H, J=11.9, 2.3 Hz, H-6), 3.78 (dd, 1H, J=11.8, 4.4 Hz, H-6), 3.46 (dd, 1H, J=9.7, 9.3 Hz, H-4), 3.13 (dd, 1H, J=10.5, 3.7 Hz, H-2); ‘Ring II’: δ_{H}=3.80 (t, 1H, J=8.8 Hz, H-5), 3.77-3.67 (m, 3H, H-1, H-3, H-4), 3.56 (t, 1H, J=9.6 Hz, H-6), 2.59-2.48 (m, 1H), 1.68 (dd, 1H, J=26.3, 12.7 Hz, H-2).

¹³C NMR (125 MHz, MeOD): δ_{C}=99.3 (C1′), 80.7, 77.8 (C5), 77.7 (C6), 73.9 (C5′), 72.4 (C3′), 71.6, 64.8 (C2′), 62.1 (C6′), 61.6, 60.9, 33.1 (C2).

MALDI TOFMS: calculated for C₁₂H₁₉N₉O₇ ([M+K]⁺) m/e 440.3; measured m/e 440.2).

### Preparation of 4′,6′-O-benzylidene-1,2′,3-triazido-paromamine (42

Compound 18 (1 gram, 2.49 mmol) was dissolved in dry DMF (20 mL) and Benzaldehyde dimethyl acetal (0.87 mL, 5.79 mmol) and a catalytic amount of CSA were added. The reaction mixture was stirred at 60° C. and the reaction progress was monitored by TLC (EtOAc 60%, Hexane 40%), which indicated the completion of the reaction after 2 hours. The reaction mixture was diluted with EtOAc and extracted with saturated aqueous solutions of NaHCO₃ and Brine. The combined organic layer was dried over MgSO₄, filtered and concentrated under reduced pressure. The crude product was purified by flash chromatography (EtOAc/hexane 1:1) to afford Compound 42 (1.0 gram, 8 3% yield).

¹H NMR (600 MHz, MeOD): ‘Ring I’: δ_{H}=5.69 (d, 1H, J=3.5 Hz, H-1), 4.27 (dd, 1H, J₁=10.0, J₂=5.0 Hz, H-6), 4.20 (td, 1H, J₁=10.1, J₂=5.0 Hz, H-6), 4.15 (t, 1H, J=9.7 Hz, H-3), 3.81 (t, 1H, J=10.1 Hz, H-5), 3.59 (t, 1H, J=9.56 Hz, H-4), 3.31 (dd, 1H, J₁=10.4, J₂=4.6 Hz, H-2); ‘Ring II’: δ_{H}=3.57 (t, 1H, J=8.2 Hz, H-5), 3.54-3.48 (m, 2H, H-3, H-4), 3.45 (ddd, 1H, J=14.7, 11.3, 5.5 Hz, H-1), 3.31 (t, 1H, J=9.7 Hz, H-6), 2.28 (dt, 1H, J₁=8.5, J₂=3.9 Hz, H-2eq), 1.46 (ddd, 1H, J₁=J₂=J₃=12.3 Hz, H-2ax); the additional peaks in the spectrum were identified as follow: 7.58-7.50 (m, 2H), 7.43-7.35 (m, 3H, Ar), 7.43-7.35 (m, 3H, Ar), 5.63 (s, 1H, PhCH).

¹³C NMR (150 MHz, MeOD): δ_{C}=139.10 (Ar), 129.98 (Ar), 129.07 (Ar), 127.56 (Ar), 103.14 (PhCH), 100.36 (C-1′), 83.06, 81.33, 77.82, 77.81, 69.85 (C-5′), 69.59 (C-6′), 65.23 (s), 64.58 (s), 61.76 (s), 60.95 (s), 33.21 (C-2).

MALDI TOFMS: calculated for C₁₉H₂₄N₉O₇ ([M+H]+) m/e 490.4; measured m/e 490.0.

### Preparation of 4′,6′-O-benzylidene-1,2′,3-triazido-peracetylparomamine (43)

Compound 42 (1.4 gram, 2.94 mmol) was dissolved in anhydrous pyridine (8 mL) and Acetic anhydride (1.4 mL, 14.8 mmol), and 4-DMAP (3.2 grams, 26.1 mmol) was added. The reaction progress was monitored by TLC, which indicated completion after 4 hours. The reaction mixture was diluted with EtOAc, and extracted with aqueous solution of HCl (2%), saturated aqueous NaHCO₃, and brine. The combined organic layers were dried over anhydrous MgSO₄ and concentrated. The crude product was purified by silica gel column chromatography (EtOAc/Hexane 4:6) to afford 43 (1.32 gram, 73% yield).

¹H NMR (600 MHz, MeOD): ‘Ring I’: δ_{H}=5.57 (dd, 1H, J₁=10.3, J₂=9.6 Hz, H-3), 5.15 (d, 1H, J=3.2 Hz, H-1), 4.31 (dt, 2H, J₁=13.0, J₂=5.0 Hz, H-5, H-6), 3.73 (dd, 1H, J₁=14.4, J₂=5.6 Hz, H-6), 3.62 (t, 1H, J=9.3 Hz, H-4), 3.24 (dd, 1H, J₁=10.5, J₂=4.0 Hz, H-2); ‘Ring II’: δ_{H}=5.17 (t, 1H, J=9.7 Hz, H-5), 4.92 (t, 1H, J=10.0 Hz, H-6), 3.74-3.56 (m, 2H, H-4, H-1), 3.46 (ddd, 1H, J₁=12.2, J₂=10.1, J₃=4.9 Hz, H-3), 2.43 (dt, 1H J₁=13.0, J₂=4.5 Hz, H-2), 1.59 (ddd, 1H, J₁=25.8, J₂=12.8 Hz, H-2); the additional peaks in the spectrum were identified as follow: δ_{H}=7.44 (dt, J₁=5.0, J₂=3.0 Hz, 2H, Ar), 7.39-7.30 (m, 3H, Ar), 5.49 (s, 1H, PhCH).

¹³C NMR (150 MHz, CDCl₃): δ_{C}=170.06 (C═O), 169.76 (C═O), 169.37 (C═O), 136.93 (Ar), 129.26 (Ar), 128.36 (Ar), 126.30 (Ar), 101.74 (PhCH), 100.22 (C-1′), 79.17 (C-4′), 78.72 (C-4), 74.27 (C-6), 73.72 (C-5), 68.69 (C-6′), 68.63 (C-3′), 63.51 (C-5′), 61.46 (C-2′), 58.29 (C-3), 57.68 (C-1), 31.77 (C-2), 20.87 (CH₃CO), 20.67 (CH₃CO), 20.64 (CH₃CO).

### Preparation of (1S,2S,3R,4S,6R)-3-(((2S,3R,4R,5S,6R)-4-acetoxy-3-azido-5-hydroxy-6-(hydroxymethyl)tetrahydro-2H-pyran-2-yl)oxy)-4, 6-diazidocyclohexane-1,2-diyl diacetate (44)

Compound 43 (1.32 gram, 2.14 mmol) was dissolved in mixture of AcOH/H₂O (5:1, 10 mL) and the solution was stirred at 60° C. overnight. After the reaction completion, as indicated by TLC, the aqueous acetic acid was removed by evaporation. The crude residue was dissolved in EtOAc, and extracted with saturated aqueous NaHCO₃, and brine. The combined organic layers were dried over anhydrous MgSO₄ and concentrated. The crude product was purified by silica gel column chromatography (EtOAc/Hexane 6:4) to afford 44 (771 mg, 68% yield).

¹H NMR (600 MHz, CDCl₃): ‘Ring I’: δ_{H}=5.28 (t, 1H, J=9.9 Hz, H-3), 5.13 (d, H, J=3.6 Hz, H-1), 4.09 (d, 1H, J=10.0 Hz, H-4), 3.95-3.79 (m, 2H, H-6, H-6), 3.67 (t, 1H, J=9.1 Hz, H-5), 3.28 (dd, 1H, J₁=10.3, J₂=3.5 Hz, H-2); ‘Ring II’: δ_{H}=5.12 (t, 1H, J=9.8 Hz, H-5), 4.91 (t, 1H, J=10.0 Hz, H-6), 3.73-3.67 (m, 1H, H-3), 3.63 (t, 1H, J=9.7 Hz, H-4), 3.52 (td, 1H, J=12.1, 4.6 Hz, H-1), 2.42 (dt, 1H, J₁=13.2, J₂=4.4 Hz, H-2), 1.59 (ddd, 1H, J₁=J₂=J₃=12.6 Hz, H-2);); the additional peaks in the spectrum were identified as follow: δ=2.15 (s, 3H, CH₃C═O), 2.11-2.02 (m, 6H, CH₃C═O).

¹³C NMR (150 MHz, CDCl₃): δ_{C}=171.78 (C═O), 170.10 (C═O), 169.73 (C═O), 99.33 (C-1′), 78.79 (C-4), 74.21 (C-6), 73.68 (C-5), 73.03 (C-3′), 72.62 (C-4′), 69.45 (C-5′), 61.64 (C-6′), 61.02 (C-2′), 58.71 (C-1), 57.65 (C-3), 31.98 (C-2), 21.06 (CH₃CO), 20.72 (CH₃CO), 20.66 (CH₃CO).

### Preparation of (1S,2S,3R,4S,6R)-3-(((2S,3R,4S)-4-acetoxy-3-azido-6-formyl-3,4-dihydro-2H-pyran-2-yl) oxy)-4,6-diazidocyclohexane-1,2-diyl diacetate (48)

To a stirred solution of compound 47 (88 mg, 0.145 mmol) in CH₂Cl₂(3 mL) at 0° C., DMP (123 mg, 0.289 mmol) was added in one portion and the resulting mixture was stirred at 0° C. for 40 minutes. Then the reaction mixture was allowed to reach room temperature and stirred for additional 3 hours. After completion of the reaction as indicated by TLC, Et₃N (0.2 mL) was added in one-pot at r.t. and mixture was stirred 30 minutes. Thereafter, the reaction mixture was diluted with EtOAc and washed with water, followed by brine. The combined organic layers were dried over anhydrous MgSO₄ and concentrated. The crude product was purified by silica gel column chromatography (EtOAc/Hexane 3:7) to afford 48 (50 mg, 68% yield).

¹H NMR (600 MHz, CDCl₃): ‘Ring I’: δ_{H}=5.93 (d, 1H, J=2.6 Hz, H-4), 5.76 (dd, 1H, J₁=9.4, J₂=2.4 Hz, H-3), 5.38 (d, 1H, J=2.6 Hz, H-1), 3.71 (dd, 1H, J₁=9.4, J₂=2.7 Hz, H-2); ‘Ring II’: δ_{H}=5.1³ (t, 1H, J=9.9 Hz, H-5), 4.90 (t, 1H, J=10.0 Hz, H-6), 3.82 (t, 1H, J=9.8 Hz, H-4), 3.60 (ddd, 1H, J₁=12.6, J₂=10.2, J₃=4.6 Hz, H-1), 3.42 (ddd, 1H, J₁=12.6, J₂=10.0, J₃=4.6 Hz, H-3), 2.31 (dt, 1H, J₁=13.5, J₂=4.6 Hz, H-2), 1.49 (ddd, 1H, J₁=J₂=J₃=12.7 Hz, H-2); the additional peaks in the spectrum were identified as follow: 6=9.24 (s, 1H, CHO), 2.14 (s, 3H, CH₃CO), 2.08 (s, 3H, CH₃CO), 2.06 (s, 3H, CH₃CO);

¹³C NMR (150 MHz, CDCl₃): δ_{C}=185.12 (CHO), 170.01 (C═O), 169.87 (C═O), 169.48 (C═O), 148.79 (C-5′), 116.71 (C-4′), 98.98 (C-1′), 79.20 (C-4), 73.99 (C-6), 73.25 (C-5), 66.43 (C-3′), 59.14 (C-3), 58.50 (C-2′), 57.84 (C-1), 32.14 (C-2), 20.91 (CH₃CO), 20.69 (CH₃CO), 20.64 (CH₃CO).

### Preparation of (1S,2S,3R,4S,6R)-3-(((2S,3R,4S)-4-acetoxy-3-azido-6-(hydroxymethyl)-3,4-dihydro-2H-pyran-2-yl) oxy)-4,6-diazidocyclohexane-1,2-diyl diacetate (49)

To a stirred solution of aldehyde 48 (1.0 gram, 1.97 mmol) in dry MeOH (10 mL), cooled to 0° C., CeCl₃.7H₂O (734 mg, 1.97 mmol) and NaBH₄ (74 mg, 1.95 mmol) were added successively. The progress of the reaction was monitored by TLC (EtOAc/Hexane 2:3), which indicated completion after 1 hour. The MeOH was evaporated completely and H₂O was added. The aqueous layer was extracted with EtOAc. The combined organic layers were washed with brine, dried over MgSO₄, evaporated to dryness, and purified by column chromatography (silica gel, EtOAc/Hexane) to yield corresponding allyl alcohol 49 (960 mg, 96%).

¹H NMR (600 MHz, CDCl₃): ‘Ring I’: δ_{H}=5.44 (d, 1H, J=5.9, H-3), 5.25 (d, 1H, J=2.4 Hz, H-1), 5.03 (d, 1H, J=2.7 Hz, H-4), 4.09-3.96 (m, 2H, H-6, H-6), 3.58 (dd, 1H, J₁=7.0, J₂=2.5 Hz, H-2); ‘Ring II’: δ_{H}=5.12 (t, 1H, J=9.9 Hz, H-5), 4.89 (t, 1H, J=10.0 Hz, H-6), 3.79 (t, 1H, J=9.8 Hz, H-4), 3.69-3.54 (m, 1H, H-1), 3.48 (ddd, 1H, J₁=12.6, J2=10.0, J₃=4.6 Hz, H-3), 2.30 (dt, 1H, J₁=13.4, J₂=4.5 Hz, H-2eq), 1.44 (ddd, 1H, J₁=J2=J3=12.8 Hz, H-2ax); The additional peaks in the spectrum were identified as follow: δ_{H}=2.57 (brs, 1H, 6′-OH), 2.06 (s, 3H, CH₃), 2.04 (s, 6H, CH₃).

¹³C NMR (150 MHz, CDCl₃): δ_{C}=170.0 (CH₃—CO), 169.9 (CH₃—CO), 169.4 (CH₃—CO), 152.6 (C5′), 98.3 (C1′), 96.3 (C4′), 78.8 (C4), 73.9 (C6), 73.3 (C5), 66.6 (C3′), 61.7 (C6′), 59.3 (C3), 58.9 (C2′), 57.8 (C1), 32.3 (C2), 21.0 (CH₃), 20.6 (CH₃), 20.5 (CH₃).

### Preparation of (1S,2R,3R,4S,6R)-4, 6-diazido-3-(((2S,3R,4S)-3-azido-4-hydroxy-6-(hydroxymethyl)-3,4-dihydro-2H-pyran-2-yl)oxy)cyclohexane-1,2-diol (50)

To a stirred solution of alcohol 49 under argon atmosphere (960 mg, 1.88 mmol) in dry MeOH (15 mL), NaOMe (459 mg, 8.49 mmol) was added. The progress of the reaction was monitored by TLC (EtOAc/Hexane 3:2), which indicated completion after 6 hours. Then the reaction mixture was passed through a pad of silica gel column and the column was washed with MeOH. The combined organic layers were evaporated to dryness, and purified by column chromatography (silica gel, EtOAc/Hexane) to yield compound 50 (700 mg, 97%).

¹H NMR (500 MHz, MeOD): ‘Ring I’: δ_{H}=5.80 (d, 1H, J=2.5 Hz, H-1), 5.03 (dt, 1H, J₁=2.5, J₂=1.0 Hz, H-4), 4.47-4.39 (m, 1H, H-3), 4.06-3.96 (m, 2H, H-6), 3.42 (dd, 1H, J₁=8.0, J₂=2.5 Hz, H-2); ‘Ring II’: δ_{H}=3.61 (t, 1H, J=9.5 Hz, H-4), 3.52 (t, 1H, J=9.5 Hz, H-5), 3.46 (ddd, 1H, J₁=12.5, J₂=9.5, J₃=4.5 Hz, H-3), 3.43-3.37 (m, 1H, H-1), 3.26 (t, 1H, J=9.5 Hz, H-6), 2.16 (dt, 1H, J₁=12.5, J₂=4.5 Hz, H-2eq), 1.29 (ddd, 1H, J₁=J2=J3=12.5 Hz, H-2ax).

¹³C NMR (125 MHz, MeOD): δ_{C}=152.6, 100.5 (C4′), 99.6 (C1′), 81.7 (C4), 77.9 (C6), 77.6 (C5), 64.9 (C3′), 63.8 (C2′), 62.0 (C1), 61.9 (C6′), 61.6 (C3), 33.7 (C2).

MALDI TOFMS: calculated for C₁₂H₁₇N₉O₆ ([M+Na]+) m/e 406.3; measured m/e 406.3.

### Preparation of (1S,2R,3R,4S,6R)-4,6-diamino-3-(((2S,3R,4S)-3-amino-4-hydroxy-6-(hydroxymethyl)-3,4-dihydro-2H-pyran-2-yl)oxy)cyclohexane-1,2-diol (NB154)

To a stirred solution of Compound 50 (256 mg, 1.0 equiv.) in a mixture of THF (3.0 mL) and aqueous NaOH (1 mM, 5.0 mL), PMe₃ (1 M solution in THF, 0.55 mL, 7.8 equiv.) was added. The progress of the reaction was monitored by TLC [CH₂C12/MeOH/H₂O/MeNH₂ (33% solution in EtOH), 10:15:6:15], which indicated completion after 3.5 hours. The reaction mixture was purified by flash chromatography on a short column of silica gel. The column was washed with the following solvents: THF (100 mL), CH₂Cl₂ (100 mL), EtOH (50 mL), and MeOH (100 mL). The product was then eluted with the mixture of 5% MeNH₂ solution (33% solution in EtOH) in 80% MeOH. Fractions containing the product were combined and evaporated under vacuum. The pure product was obtained by passing the above product through a short column of Amberlite CG50 (NH₄ form). First, the column was washed with water, then the product was eluted with a mixture of 10% NH₄OH in water to yield NB154 (184 mg, 90%).

For storage and biological tests, NB154 was converted to its sulfate salt form as follow: The free base form was dissolved in water, the pH was adjusted to 7 with H₂SO₄ (0.1 N) and lyophilized to afford the sulfate salt of NB154.

¹H NMR (500 MHz, MeOD): ‘Ring I’: δ_{H}=5.40 (d, 1H, J=2.5 Hz, H-1), 4.98 (d, 1H, J=3.0 Hz, H-4), 4.06 (dd, 1H, J₁=7.0, J2=3.0 Hz, H-3), 4.01-3.91 (m, 2H, H-6), 2.92 (dd, 1H, J₁=7.0, J₂=2.5 Hz, H-2); ‘Ring II’: δ_{H}=3.41-3.35 (m, 2H, H-4, H-5), 3.09 (t, 1H, J=9.5 Hz, H-6), 2.76-2.70 (m, 1H, H-3), 2.66 (ddd, 1H, J₁=12.5, J₂=10.0, J₃=4.5 Hz, H-1), 2.03 (dt, 1H, J₁=12.5, J₂=4.5 Hz, H-2eq), 1.24 (ddd, 1H, J₁=J2=J3=12.5 Hz, H-2ax).

¹³C NMR (125 MHz, MeOD): δ_{C}=152.6, 101.8 (C1′), 101.5 (C4′), 86.7, 78.8 (C6), 77.7, 68.0 (C3′), 62.5 (C6′), 55.6 (C2′), 52.4 (C3), 51.2 (C1), 36.6 (C2).

MALDI TOFMS: calculated for C₁₂H₂₃N₃O₆ ([M+H]⁺) m/e 306.3; measured m/e 306.8.

### Syntheses of NB158 and NB159

NB158 and NB159 were prepared as depicted in Scheme 6.

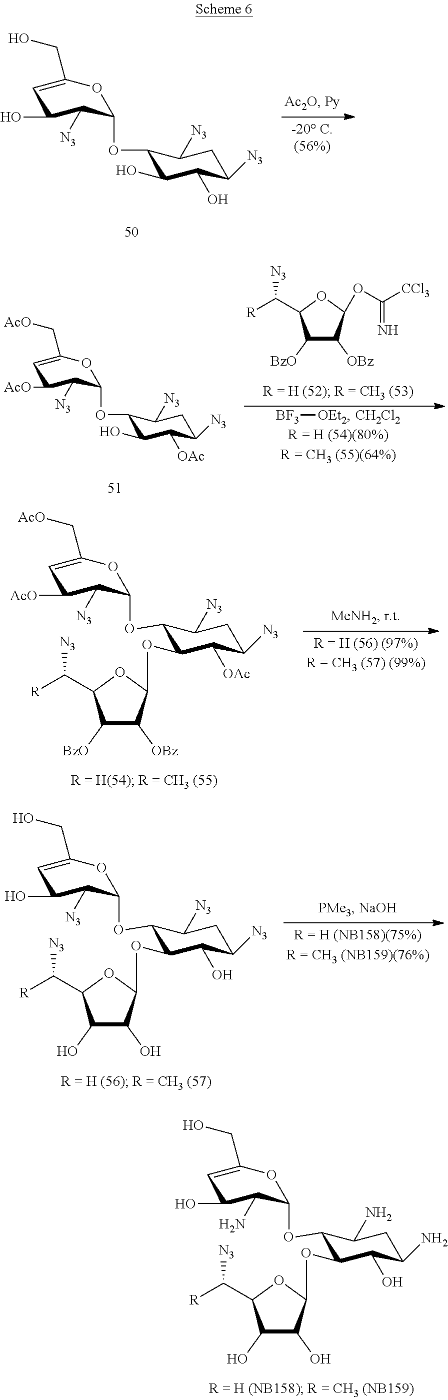

Briefly, the syntheses of pseudo-trisaccharides NB158 and NB159 were accomplished from the corresponding acceptor 51, which is obtained from regioselective acetylation of 50 at low temperature (−20° C.) using acetic anhydride in pyridine. Acceptor 51 upon glycosylation reaction with trichloroacetimidate donors 52 and 53 with catalytic amount of BF₃.OEt₂ afforded the protected pseudo-trisaccharides 54 and 55 exclusively as corresponding β-anomers in excellent yields. The global ester deprotection of pseudo-trisaccharides 54 and 55 with methylamine and the Staudinger reaction to convert azides into corresponding amines resulted in Compounds NB158 and NB159.

### Preparation of ((2S,3R,4S)-4-acetoxy-2-(((1R,2S,3S,4R,6S)-3-acetoxy-4,6-diazido-2-hydroxycyclohexyl)oxy)-3-azido-3,4-dihydro-2H-pyran-6-yl)methyl acetate (51)

Compound 50 (700 mg, 1.82 mmol) was dissolved in anhydrous pyridine (8 mL) and cooled to −20° C. At this temperature, acetic anhydride (0.6 mL, 6.19 mmol) was added dropwise and the reaction was allowed to progress at −20° C. The reaction progress was monitored by TLC, which indicated completion after 17 hours. The reaction mixture was diluted with EtOAc, and extracted with aqueous solution of NaHCO₃, HCl (2%), saturated aqueous NaHCO₃, and brine. The combined organic layers were dried over anhydrous MgSO₄ and concentrated. The crude product was purified by silica gel column chromatography to afford 51 (520 mg, 56%).

¹H NMR (600 MHz, CDCl₃): ‘Ring I’: δ_{H}=5.62 (d, 1H, J=8.7, H-3), 5.59 (d, 1H, J=2.8 Hz, H-1), 5.03 (d, 1H, J=2.7 Hz, H-4), 4.52 (q, 2H, J=13.4 Hz, H-6, H-6), 3.77 (dd, 1H, J₁=8.7, J₂=2.8 Hz, H-2); ‘Ring II’: δ_{H}=4.86 (t, 1H, J=9.9 Hz, H-6), 3.69 (td, 1H, J₁=9.5, J₂=4.3 Hz, H-5), 3.58 (t, 1H, J=9.5 Hz, H-4), 3.50 (ddd, 1H, J₁=12.6, J₂=10.0, J₃=4.6 Hz, H-1), 3.37 (ddd, 1H, J₁=12.6, J₂=9.8, J₃=4.6 Hz, H-3), 2.28 (dt, 1H, J₁=13.5, J₂=4.6 Hz, H-2eq), 1.43 (ddd, 1H, J₁=J2=J3=12.6 Hz, H-2ax); The additional peaks in the spectrum were identified as follow: δ_{H}=2.17 (s, 3H, CH₃), 2.12 (s, 3H, CH₃), 2.10 (s, 3H, CH₃).

¹³C NMR (150 MHz, CDCl₃): δ_{C}=170.9 (CH₃—CO), 170.4 (CH₃—CO), 170.4 (CH₃—CO), 148.2 (C5′), 99.1 (C4′), 98.8 (C1′), 83.1 (C4), 75.7 (C6), 74.7 (C5), 67.4 (C3′), 62.4 (C6′), 59.7 (C2′), 59.1 (C3), 58.0 (C1), 32.6 (C2), 21.1 (CH₃), 20.9 (CH₃), 20.9 (CH₃).

### Preparation of Glycosylation Product (54)

Anhydrous CH₂Cl₂ (15 mL) was added to a powdered, flame-dried 4 Å molecular sieves (2.0 grams), followed by the addition of acceptor 51 (270 mg, 0.53 mmol) and donor 52 (1.115 gram, 2.11 mmol). The reaction mixture was stirred for 10 minutes at room temperature and was then cooled to −30° C. At this temperature, catalytic amount of BF₃.Et₂O (0.1 ml) was added and the mixture was stirred at −30° C. and the reaction progress was monitored by TLC, which indicated the completion after 60 minutes. The reaction mixture was diluted with ethyl acetate and washed with saturated NaHCO₃ and brine. The combined organic layer was dried over MgSO₄, evaporated and subjected to column chromatography (EtOAc/Hexane) to obtain Compound 54 (370 mg) in 80% yield.

¹H NMR (600 MHz, CDCl₃): ‘Ring I’: δ_{H}=5.69 (d, 1H, J=2.3, H-1), 5.43 (dd, 1H, J₁=6.4, J₂=4.0 Hz, H-3), 5.07 (d, 1H, J=3.3 Hz, H-4), 4.55 (q, 2H, J=13.3 Hz, H-6, H-6), 3.92 (dd, 1H, J₁=6.8, J₂=2.3 Hz, H-2); ‘Ring II’: δ_{H}=5.0 (t, 1H, J=10.1 Hz, H-6), 3.87 (t, 1H, J=9.4 Hz, H-5), 3.79 (t, 1H, J=9.6 Hz, H-4), 3.49 (ddd, 1H, J₁=12.2, J₂=10.0, J₃=4.3 Hz, H-1), 3.43 (ddd, 1H, J₁=12.1, J₂=9.8, J₃=4.5 Hz, H-3), 2.34-2.22 (m, 1H, H-2eq), 1.45 (ddd, 1H, J₁=J2=J3=12.7 Hz, H-2ax); ‘Ring III’: δ_{H}=5.56 (d, 1H, J=1.1 Hz, H-1), 5.55-5.53 (m, 1H, H-2), 5.44 (dd, 1H, J₁=6.8, J₂=5.3 Hz, H-3), 4.57-4.49 (m, 1H, H-4), 3.66 (dd, 1H, J₁=13.5, J₂=3.6 Hz, H-5), 3.56 (dd, 1H, J₁=13.3, J₂=6.0 Hz, H-5); The additional peaks in the spectrum were identified as follow: H=7.93 (t, 2H, J=4.2 Hz, Ar), 7.88 (dd, 2H, J₁=8.3, J₂=1.2 Hz, Ar), 7.59-7.50 (m, 2H, Ar), 7.39 (t, 2H, J=7.9 Hz, Ar), 7.34 (t, 2H, J=7.9 Hz, Ar), 2.29 (s, 3H, CH₃), 2.10 (s, 3H, CH₃), 2.09 (s, 3H, CH₃).

¹³C NMR (150 MHz, CDCl₃): δ_{C}=170.3 (CH₃—CO), 170.1 (CH₃—CO), 170.0 (CH₃—CO), 165.5 (C₆H₅-CO), 165.2 (C₆H₅-CO), 149.3 (C5′), 133.8 (Ar), 133.7 (Ar), 129.7 (Ar), 129.7 (Ar), 128.8 (Ar), 128.7 (Ar), 128.6 (Ar), 128.5 (Ar), 107.5 (C1″), 97.9 (C1′), 97.8 (C4′), 80.8 (C4″, C4), 78.9 (C5), 74.7 (C2″), 73.9 (C6), 71.7 (C3′), 66.8 (C3″), 62.3 (C6′), 59.8 (C3), 59.3 (C2′), 58.4 (C1), 52.7 (C5″), 32.5 (C2), 21.1 (CH₃), 20.9 (CH₃), 20.8 (CH₃).

### Preparation of Compound 56

The glycosylation product 54 (370 mg, 0.422 mmol) was treated with a solution of MeNH₂ (33% solution in EtOH, 15 mL) and the reaction progress was monitored by TLC (EtOAc/MeOH 85:15), which indicated completion after 12 hours. The reaction mixture was evaporated to dryness and was subjected to column chromatography (MeOH/EtOAc 2:8) to obtain the corresponding completely unprotected perazido derivative 56 (237 mg) in 97% yield.

¹H NMR (600 MHz, MeOD): ‘Ring I’: δ_{H}=5.83 (d, 1H, J=2.5, H-1), 5.02 (dd, 1H, J₁=1.8, J₂=1.1 Hz, H-4), 4.35 (dd, 1H, J₁=4.4, J₂=2.4 Hz, H-3), 4.05-3.94 (m, 2H, H-6, H-6), 3.53 (dd, 1H, J₁=7.6, J₂=4.2 Hz, H-2); ‘Ring II’: δ_{H}=3.70 (t, 1H, J=9.7 Hz, H-4), 3.62 (t, 1H, J=9.1 Hz, H-5), 3.49-3.41 (m, 1H, H-3), 3.39 (dt, 1H, J₁=9.8, J₂=4.9 Hz, H-1), 3.37-3.34 (m, 1H, H-6), 2.12 (dt, 1H, J₁=13.0, J₂=4.5 Hz, H-2eq), 1.23 (ddd, 1H, J₁=J₂=J₃=12.5 Hz, H-2ax); ‘Ring III’: δ_{H}=5.37 (d, 1H, J=1.3 Hz, H-1), 4.16 (dd, 1H, J₁=4.7, J₂=1.3 Hz, H-2), 4.10 (dd, 1H, J₁=7.7, J₂=4.2 Hz, H-3), 4.02 (dd, 1H, J₁=7.0, J₂=2.7 Hz, H-4), 3.59 (dd, 1H, J₁=13.3, J₂=3.2 Hz, H-5), 3.50 (dd, 1H, J₁=13.2, J₂=6.4 Hz, H-5);

¹³C NMR (150 MHz, MeOD): δ_{C}=152.8 (C5′), 111.1 (C1″), 100.1 (C4′), 98.8 (C1′), 83.9 (C5), 82.4 (C4″), 79.7 (C4), 77.5 (C6), 76.2 (C2″), 72.4 (C3″), 65.3 (C3′), 64.0 (C2′), 62.1 (C6′), 61.9 (C1), 61.7 (C3), 54.2 (C5″), 33.5 (C2).

### Preparation of NB158

To a stirred solution of compound 56 (237 mg, 0.438 mmol) in a mixture of THF (3 mL) and aqueous NaOH (1 mM, 5 mL), PMe₃ (1 M solution in THF, 3.5 mL, 40.1 mmol) was added. The progress of the reaction was monitored by TLC [CH₂Cl₂/MeOH/H₂O/MeNH₂ (33% solution in EtOH), 10:15:6:15], which indicated completion after 3 hours. The reaction mixture was purified by flash chromatography on a short column of silica gel. The column was washed with the following solvents: THF (100 mL), CH₂Cl₂ (100 mL), EtOH (50 mL), and MeOH (100 mL). The product was then eluted with the mixture of 5% MeNH₂ solution (33% solution in EtOH) in 80% MeOH. Fractions containing the product were combined and evaporated under vacuum. The pure product was obtained by passing the above product through a short column of Amberlite CG50 (NH₄⁺ form). First, the column was washed with water, then the product was eluted with a mixture of 10% NH₄OH in water to yield NB158 (138 mg, 75%).

For storage and biological tests, NB158 was converted to its sulfate salt form as follow: The free base form was dissolved in water, the pH was adjusted to 6.7 with H₂SO₄ (0.1 N) and lyophilized to afford the sulfate salt of NB158.

¹H NMR (600 MHz, MeOD): ‘Ring I’: δ_{H}=5.40 (d, 1H, J=2.0, H-1), 5.01 (d, 1H, J=3.7 Hz, H-4), 4.04 (t, 1H, J=5.3 Hz, H-3), 4.0 (s, 2H, H-6, H-6), 3.09 (dd, 1H, J₁=5.1, J₂=1.9 Hz, H-2); ‘Ring II’: δ_{H}=3.57-3.50 (m, 2H, H-4, H-5), 3.19 (t, 1H, J=9.1 Hz, H-6), 2.79 (ddd, 1H, J₁=12.5, J2=9.3, J3=4.3 Hz, H-3), 2.67 (ddd, 1H, J₁=11.8, J₂=9.9, J₃=4.1 Hz, H-1), 2.04 (dt, 1H, J₁=8.3, J₂=6.2 Hz, H-2eq), 1.24 (ddd, 1H, J₁=J2=J3=12.3 Hz, H-2ax); ‘Ring III’: δ_{H}=5.29 (s, 1H, H-1), 4.14 (d, 1H, J=5.4 Hz, H-2), 4.06-4.02 (m, 1H, H-3), 3.92-3.87 (m, 1H, H-4), 2.98 (dd, 1H, J₁=13.0, J2=4.4 Hz, H-5), 2.84 (dd, 1H, J₁=12.9, J₂=8.4 Hz, H-5);

¹³C NMR (150 MHz, MeOD): δ_{C}=153.4 (C5′), 110.6 (C1″), 100.5 (C4′, C1′), 84.9 (C5), 84.45 (C4), 84.41 (C4″), 78.9 (C6), 76.3 (C2″), 72.8 (C3″), 67.8 (C3′), 62.3 (C6′), 55.0 (C2′), 52.5 (C1), 51.4 (C3), 45.4 (C5″), 37.2 (C2).

### Preparation of Glycosylation Product (55)

Anhydrous CH₂Cl₂ (15 mL) was added to a powdered, flame-dried 4 Å molecular sieves (2.0 grams), followed by the addition of acceptor 51 (265 mg, 0.520 mmol) and donor 53 (1.12 gram, 2.06 mmol). The reaction mixture was stirred for 10 minutes at room temperature and was then cooled to −30° C. At this temperature, catalytic amount of BF₃.Et₂O (0.1 ml) was added and the mixture was stirred at −30° C. and the reaction progress was monitored by TLC, which indicated the completion after 60 minutes. The reaction mixture was diluted with ethyl acetate and washed with saturated NaHCO₃ and brine. The combined organic layer was dried over MgSO₄, evaporated and subjected to column chromatography (EtOAc/Hexane) to obtain Compound 55 (295 mg) in 64% yield.

¹H NMR (600 MHz, CDCl₃): ‘Ring I’: δ_{H}=5.69 (d, 1H, J=2.4, H-1), 5.42 (dd, 1H, J₁=6.7, J₂=3.8 Hz, H-3), 5.06 (d, 1H, J=3.0 Hz, H-4), 4.54 (q, 2H, J=13.3 Hz, H-6, H-6), 3.96 (dd, 1H, J₁=6.8, J₂=2.5 Hz, H-2); ‘Ring II’: δ_{H}=4.99 (t, 1H, J=9.9 Hz, H-6), 3.87 (t, 1H, J=9.5 Hz, H-5), 3.78 (t, 1H, J=9.5 Hz, H-4), 3.50 (ddd, 1H, J₁=12.6, J2=10.1, J₃=4.6 Hz, H-1), 3.41 (ddd, 1H, J₁=12.5, J2=9.7, J₃=4.6 Hz, H-3), 2.28 (dt, 1H, J₁=13.2, J₂=4.6 Hz, H-2eq), 1.44 (ddd, 1H, J₁=J2=3=12.7 Hz, H-2ax); ‘Ring III’: δ_{H}=5.58 (s, 1H, H-1), 5.54 (d, 1H, J=4.9 Hz, H-2), 5.44 (dd, 1H, J₁=7.5, J₂=5.1 Hz, H-3), 4.31 (dd, 1H, J₁=7.1, J₂=6.0 Hz, H-4), 3.67 (p, 1H, J=6.7 Hz, H-5), 1.31 (d, 3H, J=6.8 Hz, 6-CH₃); The additional peaks in the spectrum were identified as follow: δ_{H}=7.89 (ddt, 4H, J₁=14.3, J₂=8.4, J₃=1.4 Hz, Ar), 7.57-7.50 (m, 2H, Ar), 7.40-7.32 (m, 4H, Ar), 2.35 (s, 3H, CH₃), 2.10 (s, 3H, CH₃), 2.08 (s, 3H, CH₃).

¹³C NMR (150 MHz, CDCl₃): δ_{C}=170.3 (CH₃—CO), 170.2 (CH₃—CO), 170.1 (CH₃—CO), 165.5 (C₆H₅-CO), 165.0 (C₆H₅-CO), 149.3 (C5′), 133.76 (Ar), 133.71 (Ar), 129.75 (Ar), 129.69 (Ar), 128.8 (Ar), 128.66 (Ar), 128.61 (Ar), 128.5 (Ar), 107.2 (C″), 97.88 (C1′), 97.87 (C4′), 80.7 (C4″), 81.0 (C4), 78.2 (C5), 74.6 (C2″), 73.7 (C6), 71.9 (C3′), 66.9 (C3″), 62.3 (C6′), 59.7 (C3), 59.5 (C2′), 58.8 (C5″), 58.4 (C1), 32.5 (C2), 21.08 (CH₃), 21.01 (CH₃), 20.8 (CH₃), 15.6 (6″-CH₃).

### Preparation of Compound 57

The glycosylation product 55 (295 mg, 0.331 mmol) was treated with a solution of MeNH₂ (33% solution in EtOH, 15 mL) and the reaction progress was monitored by TLC (EtOAc/MeOH 85:15), which indicated completion after 12 hours. The reaction mixture was evaporated to dryness and was subjected to column chromatography (MeOH/EtOAc 2:8) to obtain the corresponding completely unprotected perazido derivative 57 (180 mg) in 99% yield.

¹H NMR (600 MHz, MeOD): ‘Ring I’: δ_{H}=5.91 (d, 1H, J=2.6, H-1), 5.06 (d, 1H, J=2.3 Hz, H-4), 4.42 (ddt, 1H, 1=8.0, J₂=2.7, J₃=1.4, Hz, H-3), 4.07-3.99 (m, 2H, H-6, H-6), 3.55 (dd, 1H, J₁=7.9, J₂=3.6 Hz, H-2); ‘Ring II’: δ_{H}=3.74 (t, 1H, J=9.6 Hz, H-4), 3.66 (t, 1H, J=9.0 Hz, H-5), 3.47 (ddd, 2H, J₁=12.1, J₂=8.2, J₃=3.3 Hz, H-1, H-3), 3.42-3.40 (m, 1H, H-6), 2.17 (dt, 1H, J₁=13.2, J₂=4.4 Hz, H-2eq), 1.28 (ddd, 1H, J₁=J₂=J₃=12.3 Hz, H-2ax); ‘Ring III’: δ_{H}=5.41 (d, 1H, J=1.9 Hz, H-1), 4.22-4.18 (m, 2H, H-2, H-3), 3.81 (dd, 1H, J₁=9.2, J₂=3.2 Hz, H-4), 3.72-3.66 (m, 1H, H-5), 1.40 (d, 3H, J=6.8 Hz, 6-CH₃);

¹³C NMR (150 MHz, MeOD): δ_{C}=152.5 (C5′), 110.5 (C1″), 100.3 (C4′), 98.6 (C1′), 86.3 (C4″), 83.4 (C4), 79.4 (C4), 77.4 (C6), 76.2 (C2″), 72.6 (C3″), 65.3 (C3′), 64.0 (C2′), 62.1 (C6′), 61.9 (C1), 61.7 (C3), 60.6 (C5″), 33.5 (C2), 16.0 (6″-CH₃).

### Preparation of NB159

To a stirred solution of Compound 57 (180 mg, 0.324 mmol) in a mixture of THF (3 mL) and aqueous NaOH (1 mM, 5 mL), PMe₃ (1 M solution in THF, 3.5 mL, 40.1 mmol) was added. The progress of the reaction was monitored by TLC [CH₂C12/MeOH/H₂O/MeNH₂ (33% solution in EtOH), 10:15:6:15], which indicated completion after 3 hours. The reaction mixture was purified by flash chromatography on a short column of silica gel. The column was washed with the following solvents: THF (100 mL), CH₂Cl₂ (100 mL), EtOH (50 mL), and MeOH (100 mL). The product was then eluted with the mixture of 5% MeNH₂ solution (33% solution in EtOH) in 80% MeOH. Fractions containing the product were combined and evaporated under vacuum. The pure product was obtained by passing the above product through a short column of Amberlite CG50(NH₄₊ form). First, the column was washed with water, then the product was eluted with a mixture of 10% NH₄OH in water to yield NB159 (110 mg, 76%).

For storage and biological tests, NB159 was converted to its sulfate salt form as follow: The free base form was dissolved in water, the pH was adjusted to 6.7 with H₂SO₄ (0.1 N) and lyophilized to afford the sulfate salt of NB159.

¹H NMR (600 MHz, MeOD): ‘Ring I’: δ_{H}=5.39 (s, 1H, H-1), 5.01 (d, 1H, J=3.4 Hz, H-4), 4.02 (t, 1H, J=4.0 Hz, H-3), 4.0 (d, 2H, J=2.7 H-6, H-6), 3.07 (d, 1H, J=6.2 Hz, H-2); ‘Ring II’: δ_{H}=3.54 (dd, 2H, J₁=20.3, J₂=10.7 Hz, H-4, H-5), 3.18 (t, 1H, J=9.3 Hz, H-6), 2.79 (ddd, 1H, J₁=12.8, J₂=6.9, J₃=4.0 Hz, H-3), 2.67 (ddd, 1H, J₁=9.6, J2=5.1, J3=3.9 Hz, H-1), 2.04 (dt, 1H, J₁=13.1, J₂=4.3 Hz, H-2eq), 1.24 (ddd, 1H, J₁=J₂=J₃=12.3 Hz, H-2ax); ‘Ring III’: δ_{H}=5.29 (s, 1H, H-1), 4.11 (dd, 1H, J=14.7, J₂=6.2 Hz, H-2, H-3), 3.59-3.54 (m, 1H, H-4), 2.98 (t, 1H, J=5.8 Hz, H-5), 1.19 (d, 3H, J=7.9 Hz, 6-CH₃);

¹³C NMR (150 MHz, MeOD): δ_{C}=153.4 (C5′), 109.8 (C1″), 100.4 (C1′), 100.3 (C4′), 88.5 (C4″), 84.5 (C4), 84.0 (C5), 78.8 (C6), 76.4 (C2″), 72.9 (C3″), 67.8 (C3′), 62.4 (C6′), 55.0 (C2′), 52.6 (C1), 51.4 (C3), 51.3 (C5″), 37.2 (C2), 18.9 (6″-CH₃).

### Readthrough Activity

Preliminary comparative in-vitro PTC suppression activity assays, performed essentially as described herein, showed that NB154 had readthrough activity almost 3.5 fold higher than paromamine and more or less similar activity as that of NB82.

Comparative in-vitro PTC suppression activity assays, performed essentially as described herein, further showed that NB158 and NB159 both exhibit a similar or slightly lower activity compared to their corresponding structurally related compounds NB30 and NB118.

However, the measured prokaryotic protein synthesis inhibition and subsequently the antibacterial activity of NB154, NB158 and NB159 are significantly lower than that of the corresponding paromamine, NB30, and NB118, as shown in Table 4 below, suggesting that these compounds are likely to exhibit extremely low toxicity.

TABLE 4

| | | | Antibacterial activity | |
|---|---|---|---|---|
| | | | MIC (μg/mL) | |
| | Translation Inhibition | | E. coli | B. subtilis |
| Compound | IC₅₀^{Euk} (μM) | IC₅₀^{Pro} (μM) | R477/100 | ATCC6633 |
| Paro. | 760 ± 79 | 14 ± 1.2 | — | — |
| NB154 | 375.94 ± 38.6 | 82.3 ± 11.93 | >384 | >384 |
| NB30 | 31 ± 4 | 0.45 ± 0.03 | 790 | 100 |
| NB158 | 70.7 ± 2.4 | 36.4 ± 3.5 | >192 | 192 |
| NB118 | 15.5 ± 1.3 | 1.9 ± 0.2 | 2659 | 83 |
| NB159 | 47.9 ± 3.3 | 134.5 ± 2.8 | >192 | 192 |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1-43. (canceled)

44. A compound represented by general formula I: 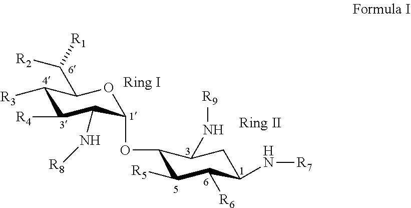
or a pharmaceutically acceptable salt thereof,
wherein:
the dashed lines indicates a stereo-configuration of position 6′ being an R configuration or an S configuration;
R₁ is selected from a hydroxy-substituted alkyl, a hydroxy-substituted alkenyl, a hydroxy-substituted cycloalkyl and a hydroxy-substituted aryl;
R₂ is selected from hydrogen, a substituted or unsubstituted alkyl and OR′, wherein R′ is selected from hydrogen, a substituted or unsubstituted alkyl, a substituted or unsubstituted cycloalkyl, a substituted or unsubstituted aryl, a substituted or unsubstituted heteroaryl, a substituted or unsubstituted alkaryl, and an acyl;
R₃-R₆ are each independently selected from hydrogen, a substituted or unsubstituted alkyl, and OR′, wherein R′ is selected from hydrogen, a monosaccharide moiety, an oligosaccharide moiety, a substituted or unsubstituted alkyl, a substituted or unsubstituted cycloalkyl, a substituted or unsubstituted aryl, a substituted or unsubstituted heteroaryl, a substituted or unsubstituted alkaryl, and an acyl; and
R₇-R₉ are each independently selected from hydrogen, acyl, an amino-substituted alpha-hydroxy acyl, a substituted or unsubstituted alkyl, a substituted or unsubstituted cycloalkyl, a substituted or unsubstituted aryl, a substituted or unsubstituted alkaryl and a cell-permealizable group.

45. The compound of claim 44, wherein R₁ is a hydroxy-substituted alkyl.

46. The compound of claim 44, wherein R₂ is OR′.

47. The compound of claim 46, wherein R′ is hydrogen.

48. The compound of claim 44, wherein R₇ is selected from the hydrogen, a substituted or unsubstituted alkyl, a substituted or unsubstituted cycloalkyl, a substituted or unsubstituted alkaryl and an amino-substituted alpha-hydroxy acyl.

49. The compound of claim 44, wherein each of R₃-R₆ is OR′ and R′ is hydrogen.

50. The compound of claim 49, being selected from:

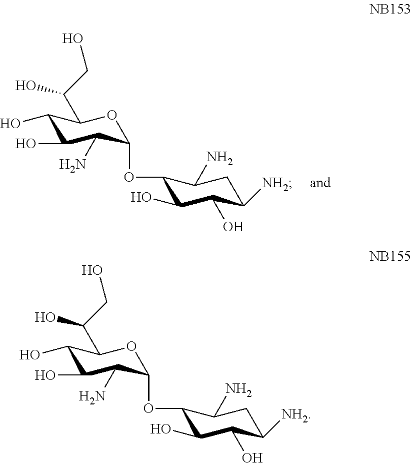

51. The compound of claim 44, wherein each of R₃-R₆ is OR′ and in at least one of R₃-R₆ R′ is a monosaccharide moiety or an oligosaccharide moiety.

52. The compound of claim 51, wherein the monosaccharide moiety is represented by Formula II: 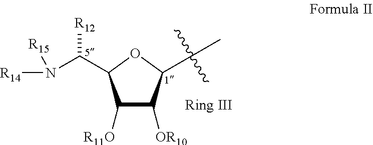
wherein the curved line denotes a position of attachment;
the dashed line indicates a stereo-configuration of position 5″ being an R configuration or an S configuration;
R₁₀ and R₁₁ are each independently selected from hydrogen, a substituted or unsubstituted alkyl, a substituted or unsubstituted cycloalkyl, a substituted or unsubstituted aryl, a substituted or unsubstituted alkaryl, and an acyl;
R₁₂ is selected from the group consisting of hydrogen, a substituted or unsubstituted alkyl, a substituted or unsubstituted cycloalkyl, and a substituted or unsubstituted aryl;
each of R₁₄ and R₁₅ is independently selected from hydrogen, acyl, an amino-substituted alpha-hydroxy acyl, a substituted or unsubstituted alkyl, a substituted or unsubstituted cycloalkyl, a substituted or unsubstituted aryl, a substituted or unsubstituted alkaryl and a cell-permealizable group, or, alternatively, R₁₄ and R₁₅ form together a heterocyclic ring.

53. The compound of claim 52, wherein R₅ is OR′ and R′ is the monosaccharide moiety, the compound being represented by Formula Ia:

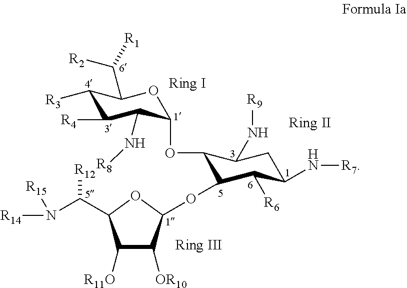

54. The compound of claim 53, being selected from:

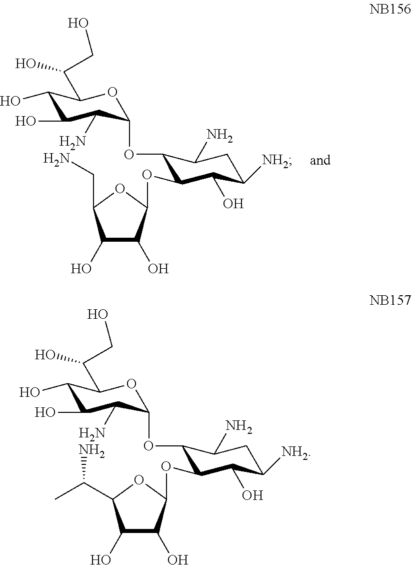

55. A pharmaceutical composition comprising the compound of claim 44 and a pharmaceutically acceptable carrier.

56. A method of treating a genetic disorder in a subject in need thereof, the method comprising administering to the subject a therapeutically effective amount of the compound of claim 44.

57. A compound represented by general formula III: 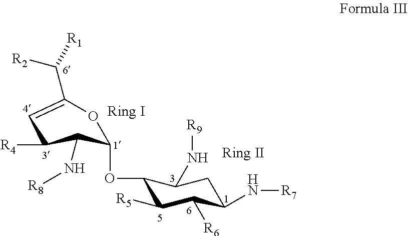
or a pharmaceutically acceptable salt thereof,
wherein:
the dashed lines indicates an optional stereo-configuration of position 6′ being an R configuration or an S configuration;
R₁ is selected from hydrogen, a substituted or unsubstituted alkyl, a substituted or unsubstituted cycloalkyl, a substituted or unsubstituted aryl;
R₂ is selected from hydrogen, a substituted or unsubstituted alkyl and OR′, wherein R′ is selected from hydrogen, a substituted or unsubstituted alkyl, a substituted or unsubstituted cycloalkyl, a substituted or unsubstituted aryl, a substituted or unsubstituted heteroaryl, a substituted or unsubstituted alkaryl, and an acyl;
R₄-R₆ are each independently selected from hydrogen, a substituted or unsubstituted alkyl, and OR′, wherein R′ is selected from hydrogen, a monosaccharide moiety, an oligosaccharide moiety, a substituted or unsubstituted alkyl, a substituted or unsubstituted cycloalkyl, a substituted or unsubstituted aryl, a substituted or unsubstituted heteroaryl, a substituted or unsubstituted alkaryl, and an acyl; and
R₇-R₉ are each independently selected from hydrogen, acyl, an amino-substituted alpha-hydroxy acyl, a substituted or unsubstituted alkyl, a substituted or unsubstituted cycloalkyl, a substituted or unsubstituted aryl, a substituted or unsubstituted alkaryl and a cell-permealizable group.

58. The compound of claim 57, wherein R₁ is hydrogen.

59. The compound of claim 57, wherein R₂ is OR′.

60. The compound of claim 57, wherein R₇ is selected from the hydrogen, a substituted or unsubstituted alkyl, a substituted or unsubstituted cycloalkyl, a substituted or unsubstituted alkaryl and an amino-substituted alpha-hydroxy acyl.

61. The compound of claim 57, wherein each of R₄-R₆ is OR′ and R′ is hydrogen.

62. The compound of claim 61, being:

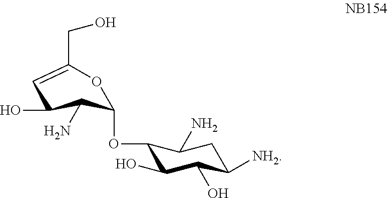

63. The compound of claim 57, wherein each of R₄-R₆ is OR′ and in at least one of R₄-R₆ R′ is a monosaccharide moiety or an oligosaccharide moiety.

64. The compound of claim 63, wherein the monosaccharide moiety is represented by Formula II: 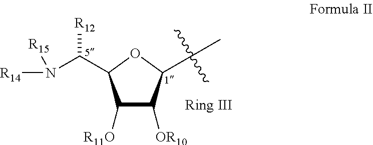
wherein the curved line denotes a position of attachment;
the dashed line indicates a stereo-configuration of position 5″ being an R configuration or an S configuration;
R₁₀ and R₁₁ are each independently selected from hydrogen, a substituted or unsubstituted alkyl, a substituted or unsubstituted cycloalkyl, a substituted or unsubstituted aryl, a substituted or unsubstituted alkaryl, and an acyl;
R₁₂ is selected from the group consisting of hydrogen, a substituted or unsubstituted alkyl, a substituted or unsubstituted cycloalkyl, and a substituted or unsubstituted aryl;
each of R₁₄ and R₁₅ is independently selected from hydrogen, acyl, an amino-substituted alpha-hydroxy acyl, a substituted or unsubstituted alkyl, a substituted or unsubstituted cycloalkyl, a substituted or unsubstituted aryl, a substituted or unsubstituted alkaryl and a cell-permealizable group, or, alternatively, R₁₄ and R₁₅ form together a heterocyclic ring.

65. The compound of claim 64, wherein R₅ is OR′ and R′ is the monosaccharide moiety, the compound being represented by Formula IIIa:

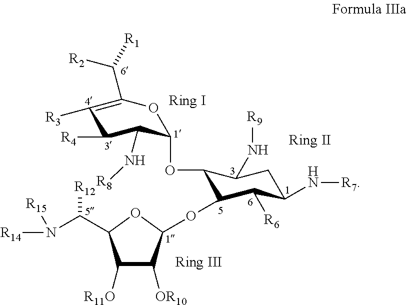

66. The compound of claim 65, being selected from:

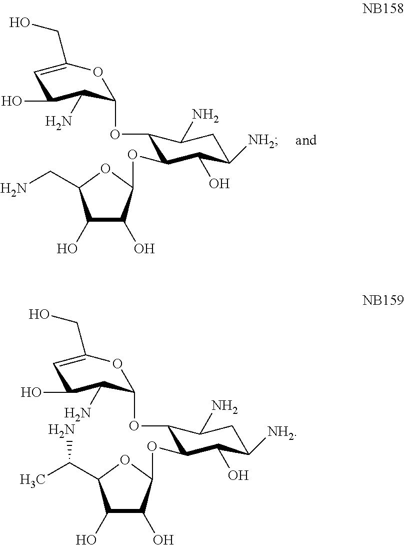

67. A pharmaceutical composition comprising the compound of claim 57 and a pharmaceutically acceptable carrier.

68. A method of treating a genetic disorder in a subject in need thereof, the method comprising administering to the subject a therapeutically effective amount of the compound of claim 57.
